(19) United States
(12) Patent Application Publication
(10) Pub. No.: US 2010/0144712 A1
(43) Pub. Date: Jun. 10, 2010

(54) TRIAZOLE DERIVATIVES HAVING ANTIFUNGAL ACTIVITY, METHOD FOR THE PREPARATION THEREOF, AND PHARMACEUTICAL COMPOSITION COMPRISING THE SAME

(75) Inventors: Bum Tae Kim, Daejeon (KR); Yong Ki Min, Daejeon (KR); Jung Nyoung Heo, Daejeon (KR); Hyuk Lee, Seoul (KR); Sung Youn Chang, Daejeon (KR); No Kyun Park, Daejeon (KR); Si-Whan Song, Yongin-si (KR); Jung-Ja Oh, Suwon-si (KR)

Correspondence Address: SUGHRUE MION, PLLC, 2100 PENNSYLVANIA AVENUE, N.W., SUITE 800, WASHINGTON, DC, 20037, US

(73) Assignees: KOREA RESEARCH INSTITUTE OF CHEMICAL TECHNOLOGY, Daejeon (KR); CHEMON INC., Suwon-si, Gyeonggi-do (KR)

(21) Appl. No.: 12/664,620

(22) PCT Filed: Jun. 12, 2008
(86) PCT No.: PCT/KR08/03281

§ 371 Date: Jan. 13, 2010

(30) Foreign Application Priority Data
Jun. 15, 2007 (KR) 10-2007-0059084

(51) Int. Cl. A61K 31/551; C07D 417/14; A61K 31/5377; A61P 31/00; A61P 43/00; A61K 31/496; C07D 401/14; C07D 413/14; A61K 31/506; C07D 405/14; C07D 513/04; C07D 403/14

(52) U.S. Cl. 514/218; 544/368; 514/233.8; 514/254.02; 544/364; 514/253.1; 544/121; 540/575; 514/252.19; 544/363; 514/253.06; 514/254.03; 544/366; 544/370; 544/295; 544/367; 514/254.07; 514/254.06

(57) ABSTRACT

A triazole derivative of formula 1 or a pharmaceutically acceptable salt, hydrate, solvate or isomer thereof is superior to the conventional antifungal drugs in antifungal activity against a wide spectrum of pathogenic fungi, and has advantageously low toxicity.

### FIELD OF THE INVENTION

The present invention relates to a novel triazole derivative having antifungal activity, a method for the preparation thereof, and a pharmaceutical composition comprising the same as an active ingredient.

### BACKGROUND OF THE INVENTION

The incidence of fungal infections in human has increased as a consequence of immune system reconstitutions such as tissue transplantation and HIV/AIDS. The fungal infections in patients with immune deficiency may lead to mental and physical disorders, mucosal diseases or death. Amphotericin B, flucytosine and a number of azole derivatives are currently available for preventing and treating diseases caused by fungal infections. However, long-term use of the above drugs has been reported to be unsatisfactory due to side effects such as less therapeutic effect, drug toxicity, poor antifungal spectrum and appearance of resistant fungi. For example, amphotericin B may cause side effects such as nephrotoxicity, hypokalaemia and anemia; and flucytosine, gene mutation and secondary drug resistance. Antifungal azole derivatives contain an azole ring having 2 or 3 nitrogen moieties, which can be classified into imidazole derivatives having 2 nitrogen moieties (e.g., ketokonazole, miconazole and clotrimazole), and triazole derivatives having 3 nitrogen moieties (e.g., itraconazole, fluconazole and voriconazole). The imidazole derivatives excepting ketokonazole have been employed for treating superficial mycosis, and the triazole derivatives have been widely used for treating superficial and deep mycoses. Ketokonazole has been shown to have potential therapeutic effects in diseases caused by Aspergillus, Candida or Cryptococcus infections, and however, it has drug toxicity and pharmacokinetic problems.

Fluconazole of Pfizer (British Pat. No. 2,099,818; and U.S. Pat. No. 4,404,216), itraconazole of Janssen (U.S. Pat. No. 4,267,179; and European Pat. Pub. No. 6,711) and voriconazole of Pfizer (European Pat. Pub. No. 440,372; and U.S. Pat. No. 5,278,175) have been known as currently available antifungal agents.

However, fluconazole widely used for treating Candida infections has been demonstrated to be ineffective in the treatments of diseases caused by infections of new fungal mutants or fluconazole resistant fungi, especially Aspergillus infection. Itraconazole effective against Aspergillus has low solubility in water and has been reported to interact with uterine cancer inducible proteins in animals. Further, voriconazole exhibits 1.6- to 160-fold greater inhibitions of ergosterol P450 than fluconazole in Candida and Aspergillus infections, and however, has limited spectrum of antifungal activity and drug toxicity.

Meanwhile, in order to mitigate increase of fluconazole resistant fungi and toxicity of oral administration, there have been numerous attempts to develop a novel compound capable of overcoming fluconazole resistance. For instance, there have been disclosed antifungal compounds in which methyl groups are introduced into side chains of triazole compounds and methods for the preparation thereof (Chem. Pharm. Bull. (2000), 48; 1947-1953; Chem. Pharm. Bull., (2000), 48, 1935-1946; U.S. Pat. No. 6,153,616; JP 2000169473; JP 2000063364; WO 9833778; and WO 9631491). However, such compounds exhibit low therapeutic effect against infectious diseases of Aspergillus and resistant fungi, and the methods for the preparation thereof are complicated.

Accordingly, the present inventors have endeavored to develop a compound having higher antifungal activity than fluconazole with low toxicity; and have unexpectedly found that a new class of triazole derivatives exhibits excellent antifungal activities against a wide spectrum of pathogenic fungi including Candida albicans, Torulopsis, Crytococcus, Aspergillus, Tricophyton and fluconazole resistant fungi with low toxicity.

### SUMMARY OF THE INVENTION

Accordingly, it is a primary object of the present invention to provide a novel compound and a pharmaceutically acceptable salt, hydrate, solvate or isomer thereof, which is superior to the conventional antifungal drugs in antifungal activity against a wide spectrum of pathogenic fungi as well as low toxicity.

It is another object of the present invention to provide a method for the preparation of said compound.

It is a further object of the present invention to provide an antifungal composition containing said compound.

In accordance with one aspect of the present invention, there is provided a novel azole derivative of formula 1 or a pharmaceutically acceptable salt, hydrate, solvate or isomer thereof:

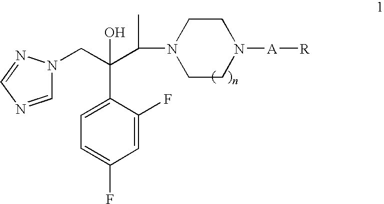

wherein,

n is 1 or 2;

A is a direct bond, C═O or CH₂; and

R is a 5 to 10-membered mono- or bi-cyclic heteroaryl ring containing 1 to 4 atoms each independently selected from the group consisting of N, O and S in its ring structure, which is substituted with one or more substituents each independently selected from the group consisting of hydrogen, halogen, hydroxy, cyano, nitro, amino, hydroxycarbonyl, C₁₋₆alkyl, C₁₋₆alkenyl, C₁₋₆alkynyl, C₁₋₆alkoxy, hydroxyl C₁₋₆alkyl, C₁₋₆alkoxy C₁₋₆alkyl, perfluoro C₁₋₆alkyl, perfluoro C₁₋₆alkoxy, C₁₋₆alkylamino, diC₁₋₆alkylamino, aminoC₁₋₆alkyl, C₁₋₆alkylamino C₁₋₆alkyl, diC₁₋₆alkylamino C₁₋₆alkyl, C₁₋₆acyl, C₁₋₆acyloxy, C₁₋₆acyloxyC₁₋₆alkyl, C₁₋₆acylamino, C₁₋₆alkylthiocarbonyl, C₁₋₆alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl, 3- to 8-membered cycloalkoxy, 3- to 8-membered cycloalkyl-C₁₋₆alkoxy, 3- to 8-membered cycloalkyl-C₁₋₆alkylamino, N—C₁₋₆alkyl N-3- to 8-membered cycloalkyl-C₁₋₆alkylamino, 4- to 8-membered heterocycloalkyl, 4- to 8-membered heterocycloalkyl-C₁₋₆alkoxy, 4- to 8-membered heterocycloalkyl-C₁₋₆alkylamino, N—C₁₋₆alkyl N-4- to 8-membered heterocycloalkyl-C₁₋₆alkylamino, heteroaryl-C₁₋₆alkyl, heteroaryl-C₁₋₆alkoxy, heteroaryl-C₁₋₆alkylamino, N—C₁₋₆alkyl N-heteroaryl-C₁₋₆alkylamino, phenyl and monocyclic heteroaryl.

### BRIEF DESCRIPTION OF THE DRAWINGS

The above and other objects and features of the present invention will become apparent from the following description of the invention, when taken in conjunction with the accompanying drawings, which respectively show:

FIG. 1: a graph showing the viabilities of mice administered with the inventive compound and then systemically infected with Aspergillus fumigatus (ATCC 16424).

### DETAILED DESCRIPTION OF THE INVENTION

In the compound of formula 1, preferred R is

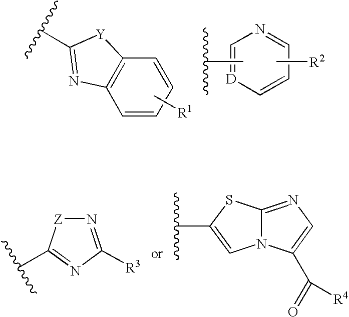

wherein,

Y is O, S, or NR⁵;

D is CH or N;

Z is O or S;

R¹ and R² are each independently hydrogen, halogen, hydroxy, cyano, nitro, amino, hydroxycarbonyl, C₁₋₆alkyl, C₁₋₆alkenyl, C₁₋₆alkynyl, C₁₋₆alkoxy, hydroxyC₁₋₆alkyl, C₁₋₆alkoxyC₁₋₆alkyl, perfluoroC₁₋₆alkyl, perfluoroC₁₋₆alkoxy, C₁₋₆alkylamino, diC₁₋₆alkylamino, aminoC₁₋₆alkyl, C₁₋₆alkylaminoC₁₋₆alkyl, diC₁₋₆alkylaminoC₁₋₆alkyl, C₁₋₆acyl, C₁₋₆acyloxy, C₁₋₆acyloxyC₁₋₆alkyl, C₁₋₆acylamino, C₁₋₆alkylthio, C₁₋₆alkylthiocarbonyl, C₁₋₆alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl, 3- to 8-membered cycloalkoxy, 3- to 8-membered cycloalkyl-C₁₋₆alkoxy, 3- to 8-membered cycloalkyl-C₁₋₆alkylamino, N—C₁₋₆alkyl N-3- to 8-membered cycloalkyl-C₁₋₆alkylamino, 4- to 8-membered heterocycloalkyl, 4- to 8-membered heterocycloalkyl-C₁₋₆alkoxy, 4- to 8-membered heterocycloalkyl-C₁₋₆alkylamino, N—C₁₋₆alkyl N-4- to 8-membered heterocycloalkyl-C₁₋₆alkylamino, heteroaryl-C₁₋₆alkyl, heteroaryl-C₁₋₆alkoxy, heteroaryl-C₁₋₄alkylamino, or N—C₁₋₆alkyl N-heteroaryl-C₁₋₆alkylamino;

R³ and R⁴ are each phenyl and monocyclic heteroaryl, substituted with one or more substituents selected from the group consisting of hydrogen, halogen, hydroxy, cyano, nitro, amino, hydroxycarbonyl, C₁₋₆alkyl, C₁₋₆alkynyl, C₁₋₆alkoxy, hydroxyC₁₋₆alkyl, C₁₋₆alkoxyC₁₋₆alkyl, perfluoroC₁₋₆alkyl and perfluoroC₁₋₆alkoxy; and

R⁵ is C₁₋₆alkenyl, C₁₋₆alkynyl, C₁₋₆alkoxyC₁₋₆alkyl or perfluoroC₁₋₆alkyl.

In the present invention, the term 5- to 10-membered mono- or bicyclic “heteroaryl” refers to furyl, thienyl, thiazolyl, pyrazolyl, isothiazolyl, oxazolyl, isooxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyridinyl, benzo[b]thiophenyl, 5,6,7,8-tetrahydro-quinolin-3-yl, benzooxazolyl, benzothiazolyl, benzisothiazolyl, benzisooxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl or benzoxazinyl.

The term “cycloalkyl” refers to cycloalkyl containing 0 to 2 unsaturated groups, such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane and norbornanyl.

The term “heterocycloalkyl” refers to pyrrolidinyl, tetrahydrofuranyl, dihydrofuranyl, tetrahydropyranyl, pyranyl, thiopyranyl, aziridinyl, oxiranyl, methylenedioxyl, chromenyl, isooxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl or chromenyl.

Representative examples of the inventive triazole derivative of formula 1 are shown in Table 1:

TABLE 1

| Ex. | Structure |
|---|---|
| 1 | 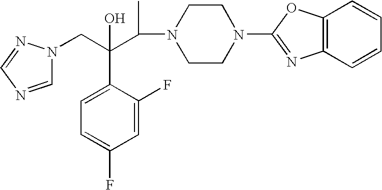 |
| 2 | 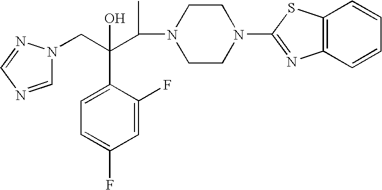 |
| 3 | 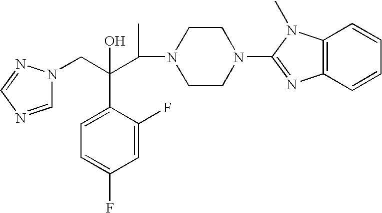 |
| 4 | 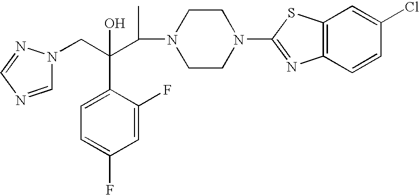 |
| 5 | 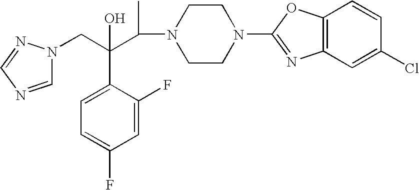 |
| 6 | 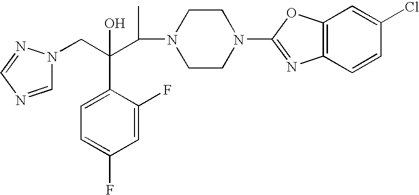 |
| 7 | 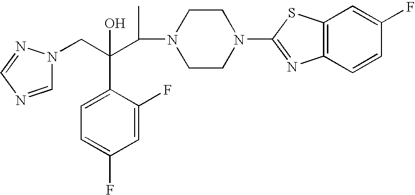 |
| 8 | 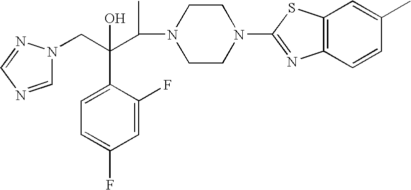 |
| 9 | 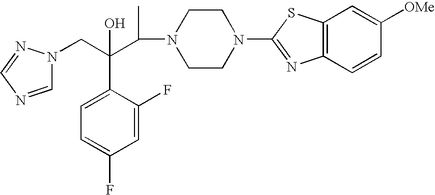 |
| 10 | 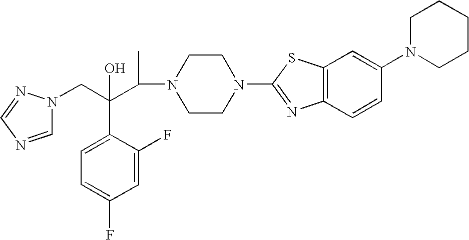 |
| 11 | 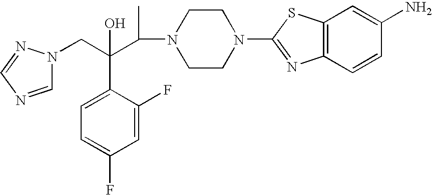 |
| 12 | 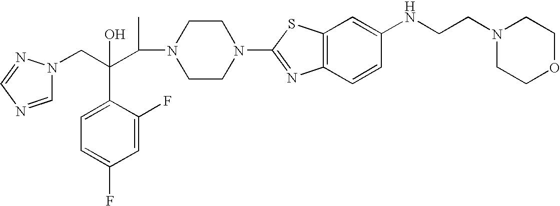 |
| 13 | 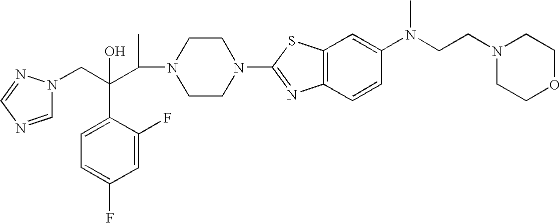 |
| 14 | 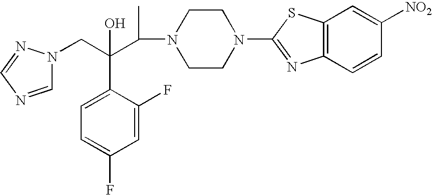 |
| 15 | 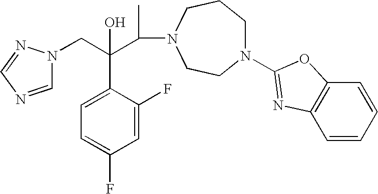 |
| 16 | 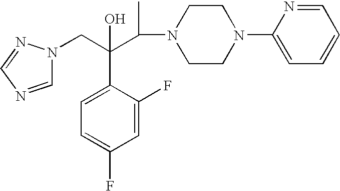 |
| 17 | 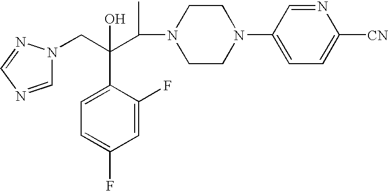 |
| 18 | 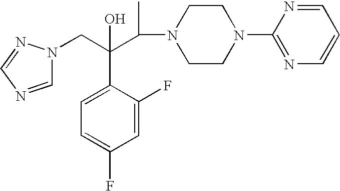 |
| 19 | 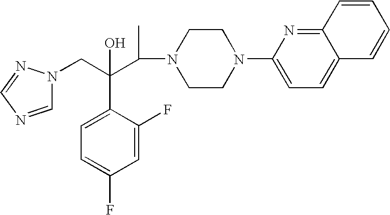 |
| 20 | 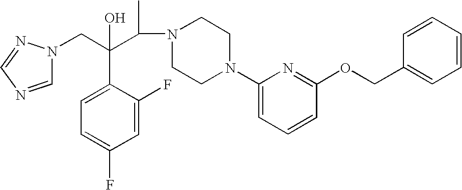 |
| 21 | 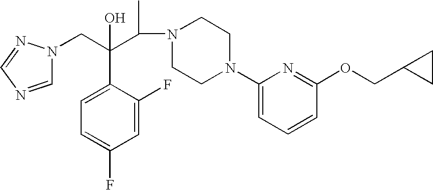 |
| 22 | 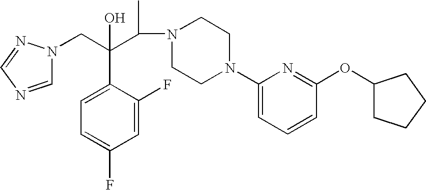 |
| 23 | 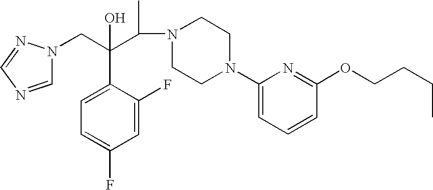 |
| 24 | 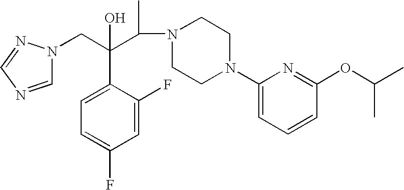 |
| 25 | 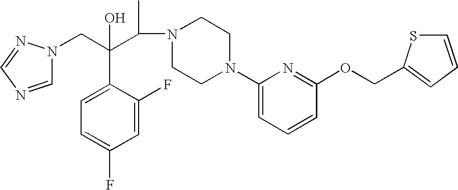 |
| 26 | 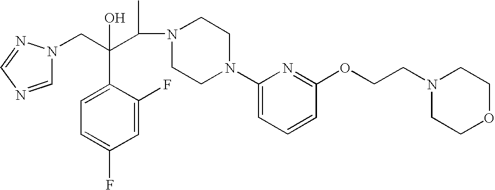 |
| 27 | 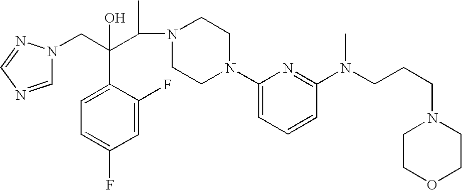 |
| 28 | 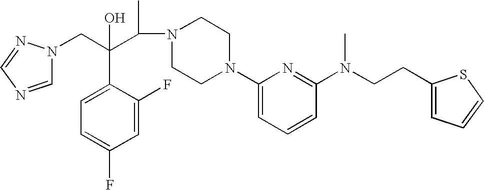 |
| 29 | 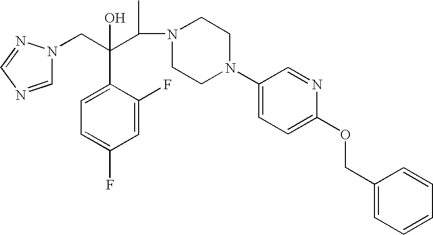 |
| 30 | 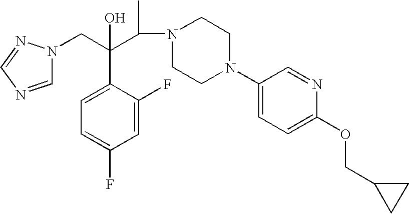 |
| 31 | 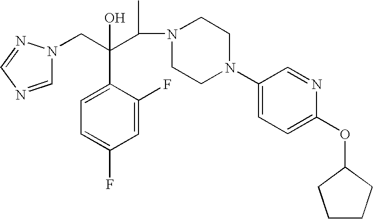 |
| 32 | 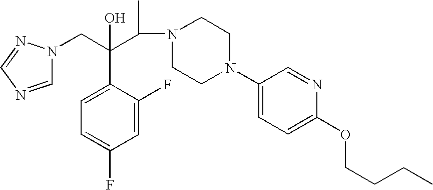 |
| 33 | 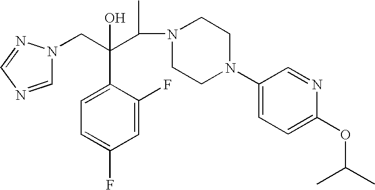 |
| 34 | 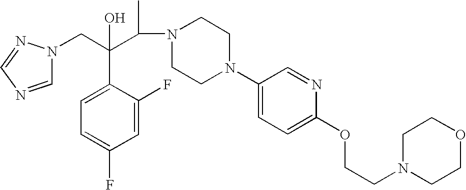 |
| 35 | 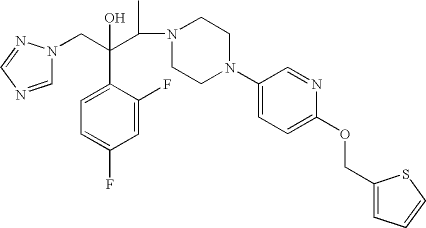 |
| 36 | 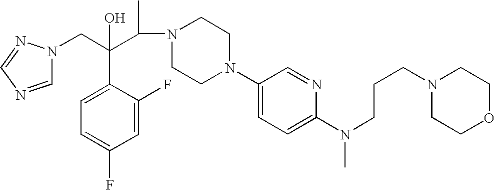 |
| 37 | 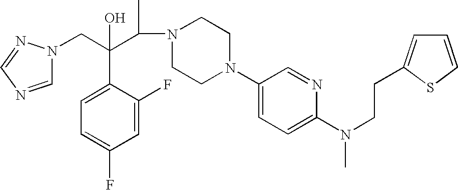 |
| 38 | 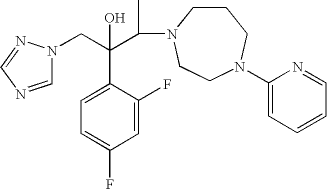 |
| 39 | 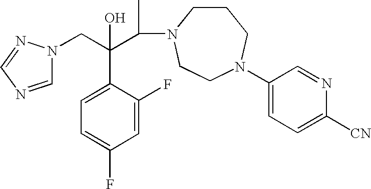 |
| 40 | 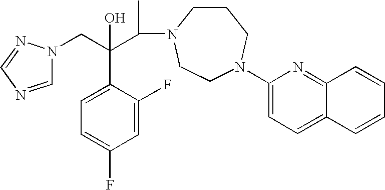 |
| 41 | 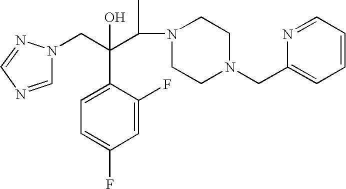 |
| 42 | 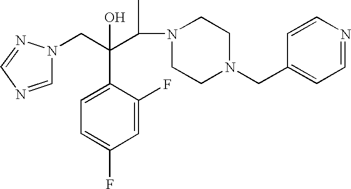 |
| 43 | 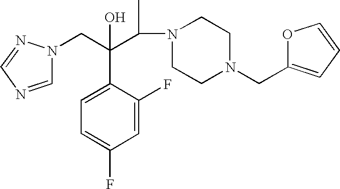 |
| 44 | 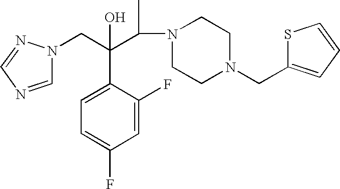 |
| 45 | 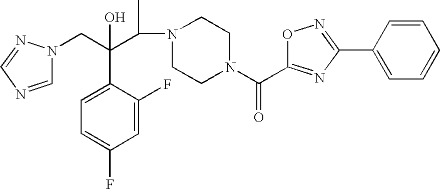 |
| 46 | 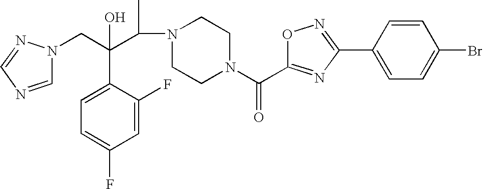 |
| 47 | 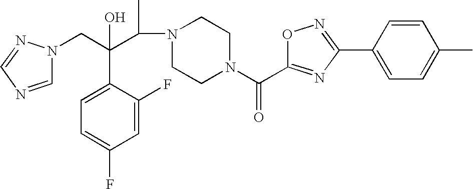 |
| 48 | 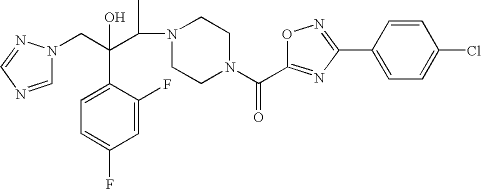 |
| 49 | 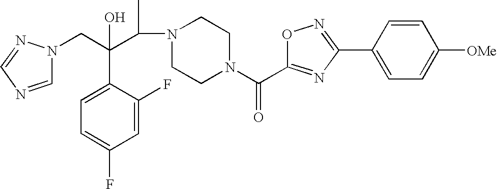 |
| 50 | 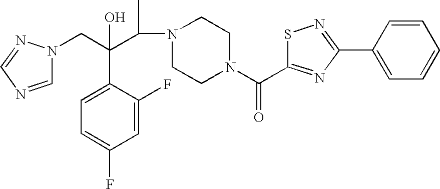 |
| 51 | 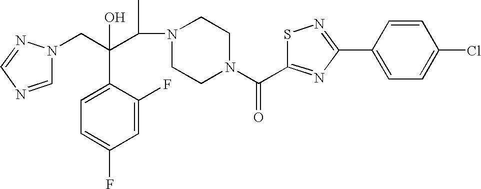 |
| 52 | 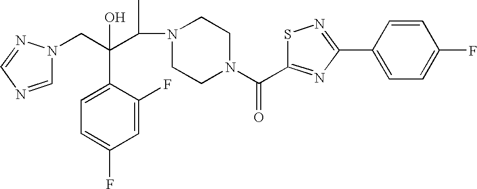 |
| 53 | 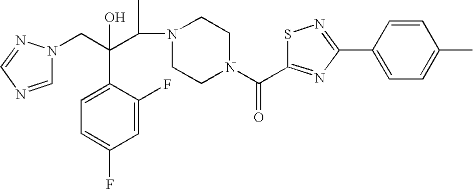 |
| 54 | 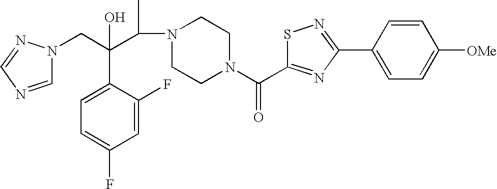 |
| 55 | 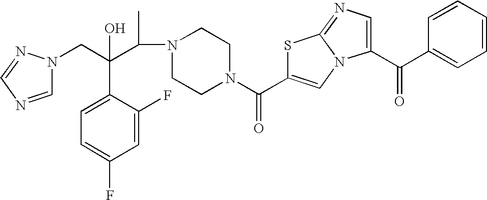 |
| 56 | 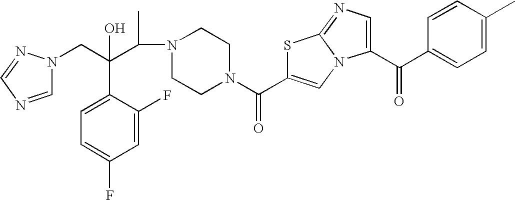 |
| 57 | 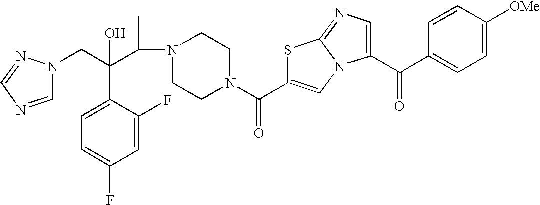 |

In the present invention, the pharmaceutically acceptable salt of the compound of formula 1 may be an acid salt derived by adding a pharmaceutically acceptable free acid. The pharmaceutically acceptable free acid may be an inorganic or organic acid, the inorganic acid may be hydrochloric acid, hydrobromic acid, sulfuric acid, sulfurous acid or phosphoric acid, and the organic acid may be citric acid, acetic acid, maleic acid, fumaric acid, gluconic acid, methanesulfonic acid, glycolic acid, succinic acid, tartaric acid, 4-toluenesulfonic acid, galacturonic acid, embonic acid, glutamic acid or aspartic acid. A preferred inorganic or organic acid may be methansulfonic acid or hydrochloric acid.

The acid salt of the present invention may be prepared by dissolving the compound of formula 1 in a water-miscible organic solvent, e.g., acetone, methanol, ethanol or acetonitrile; and reacting the mixture with an excess amount of organic acid or an aqueous inorganic acid to obtain a resulting solid, according to a conventional method. The resulting solid may be isolated from the resulting mixture by distilling and drying, or filtering the resulting mixture. Further, the present invention encompasses, within its scope, a pharmaceutically acceptable solvate or hydrate of the triazole derivative of formula 1, which may be derived from the pharmaceutically acceptable salt of the inventive triazole derivative.

Further, the triazole derivatives of the present invention may include all stereoisomers of the compound of formula 1. The compound of formula 1 of the present invention has two asymmetric carbons, which may be R- or S-isomer form, respectively, preferably all R-isomer forms. The inventive stereoisomers may be each isolated by a conventional kinetic resolution method. The inventive stereoisomers may be prepared by an asymmetrical synthesis, and isolated by conventional methods such as chromatography.

The compound of formula 1a, i.e., a compound of formula 1 wherein A is a direct bond and R is

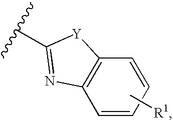

may be prepared by two methods shown in Reaction Scheme 1.

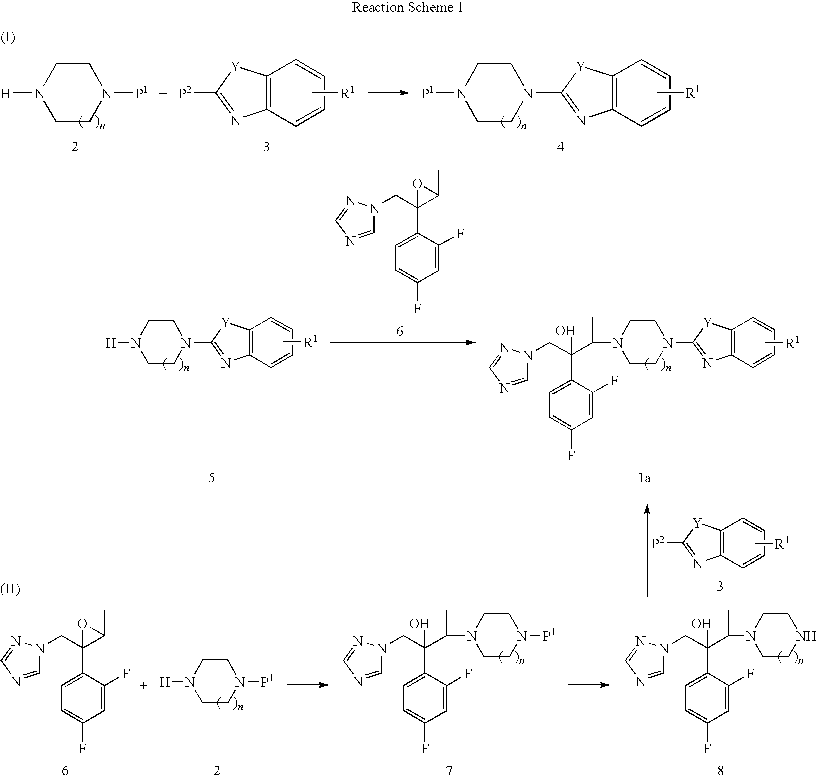

wherein,

n, Y and R¹ have the same meanings as defined above;

P¹ is hydrogen or an amine protecting group which is one of the known protecting groups described in P. G. M. Wuts and T. W. Greene, John Wiley & Sons, Protective groups in organic synthesis, 4th ed., p 696-926, preferably hydrogen, ethoxycarbonyl, t-butoxycarbonyl or benzyloxycarbonyl; and

P² is an leaving group, preferably halogen, mercapto, methanesulfonyloxy or trifluoromethanesulfonyloxy.

In the present invention, P¹ and P² have the same meanings defined above unless there are some descriptions in particular.

In Reaction Scheme 1, the compound of formula 1a may be prepared by reacting a piperazine compound of formula 2 with a hetero ring of formula 3 in the presence of a base to obtain a compound of formula 4; if P¹ in the compound of formula 4 is an amine protecting group, removing the amine protecting group from the compound of formula 4 in the presence of a base to obtain a compound of formula 5; and reacting the compound of formula 5 with an oxirane compound of formula 6. Representative examples of the base include an inorganic base such as sodium hydroxide, barium hydroxide, sodium carbonate, potassium carbonate, cesium carbonate, sodium hydrogen chloride, potassium bicarbonate, potassium phosphate, cesium fluoride, potassium fluoride and a mixture thereof; an alkali metal alkoxide such as sodium ethoxide, sodium tert-butoxide, potassium tert-butoxide and a mixture thereof; and an organic base such as N-methylmorpholine, N,N-dimethylaniline, 1,8-diaza bicyclo[5,4,0]-7-undecene (DBU), triethylamine and a mixture thereof, preferably, sodium carbonate, potassium carbonate, cesium carbonate, sodium hydrogen chloride and potassium bicarbonate, more preferably sodium carbonate and potassium carbonate.

The compound of formula 6 used as a reaction precursor in the present invention may be prepared by convention methods described in Chem. Pharm. Bull., 39, 2241-2246 (1991); Chem. Pharm. Bull., 41, 1035-1042 (1993); and Chem. Pharm. Bull., 43, 441-449 (1993). The compound of formula 6 has a chiral center, and the final product of the present invention may be controlled according to the stereoselectivity of epoxide. In one preferred embodiment of the present invention, a triazole derivative having stereoselective piperazine group of formula 1 may be prepared using a R-lactate as a starting material and (2R,3S) 2-(2,4-difluorophenyl)-3-methyl-2-(1H-1,2,4-triazol-1-yl)methyoxirane as an intermediate in accordance with a convention method (WO 1998/031675).

Further, the compound of formula 1a may be prepared by reacting the oxirane compound of formula 6 with the compound of formula 2 to obtained a chiral compound of formula 7; if P¹ is a protecting group, removing the protecting group from the compound of formula 7 to obtain a compound of formula 8; and reacting the compound of formula 8 with the hetero ring of formula 3.

In the inventive methods described above, if P¹ is hydrogen, the compounds of formulae 4 and 5 and the compounds of formulae 7 and 8 are the same compounds, respectively.

In the present invention, the reaction of (2R,3S) oxirane compound of formula 6 with the compound of formula 2 or 5 may be preferably conducted in a solvent, which is one of the known solvents which can dissolve the starting material and do not inhibit the reaction. Representative examples of the solvents may include ether-based solvents such as tetrahydrofurane, 1,2-dimethoxyethane, diethylether and dioxane; aromatic hydrocarbon-based solvents such as benzene, toluene and xylene; amide-based solvents such as N-dimethylformamide, N,N-dimethylacetamide and N-methylpyrrolidone; organic solvents such as dimethylsulfoxide, acetonitrile and propionitrile; and alcohol-based solvents such as methanol, ethanol, propanol, n-butanol and t-butanol, which may be used separately, as a mixture, or as a mixture with water. Preferred examples of the solvents may include dimethylformamide, acetonitrile, propionitrile and a mixture thereof.

Further, the above reaction may be carried out using a conventional oil bath or a professional microwave reactor, and the reaction temperature and time may be changeable according to the starting material, solvent, other reagents or equipments used in the reaction. In a preferred embodiment of the present invention, the reaction may be conducted at a temperature ranging from 60° C. to 200° C., preferably 80° C. to 120° C. for 1 to 48 hrs, preferably 6 to 12 hrs in case of using an oil bath, and at a temperature ranging from 60° C. to 200° C. for 1 min to 1 hr, preferably 10 min to 30 min together with stirring in case of using a microwave reactor.

The reaction of the compound of formula 2 or 7 with the compound of formula 3 may be conducted in the presence of the same base as described above.

Particularly, a compound of formula 1a wherein R¹ is amino may be prepared by a method shown in Reaction Scheme 2.

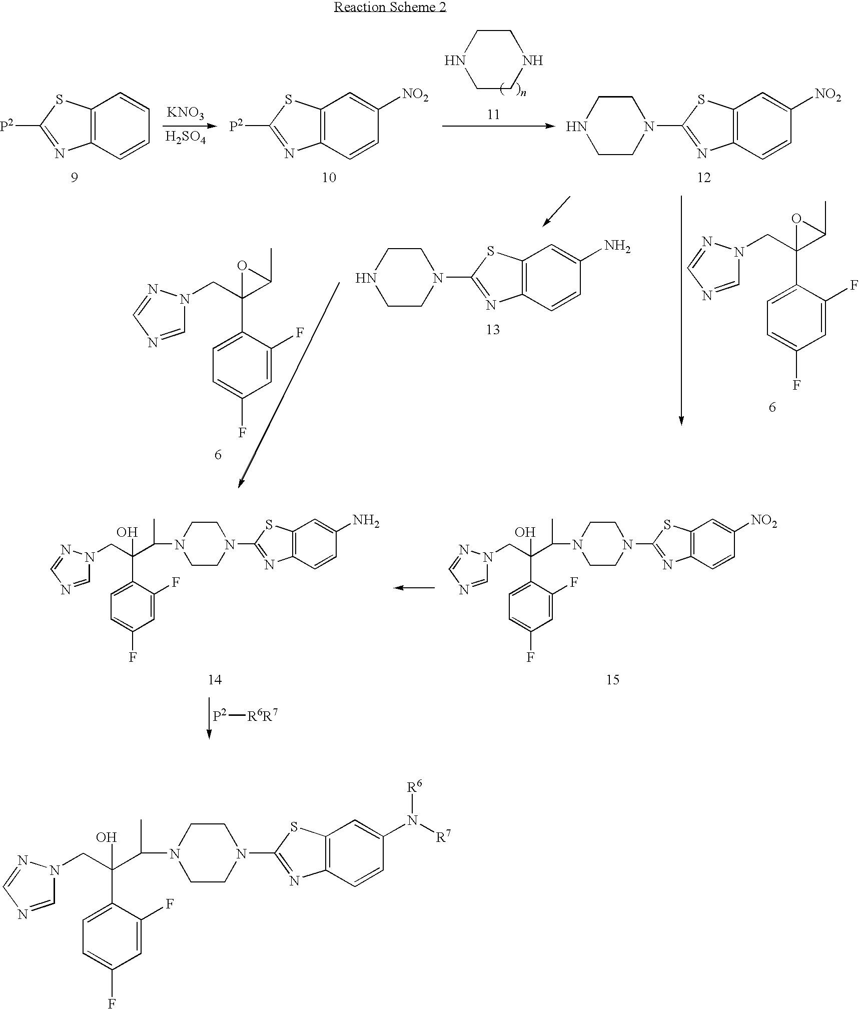

wherein,

n is 1 or 2;

P² and Y have the same meanings as defined above; and

R⁶ and R⁷ are each independently hydrogen, C₁₋₆alkyl, C₁₋₆alkenyl, C₁₋₆alkynyl, C₁₋₆alkoxy, hydroxyC₁₋₆alkyl, C₁₋₆alkoxyC₁₋₆alkyl, perfluoroC₁₋₆alkyl, perfluoroC₁₋₆alkoxy, aminoC₁₋₆alkyl, C₁₋₆alkylaminoC₁₋₆alkyl, diC₁₋₆alkylaminoC₁₋₆alkyl, C₁₋₆acyl, C₁₋₆acyloxyC₁₋₆alkyl, C₁₋₆alkylthiocarbonyl, C₁₋₆alkylthioxo, C₁₋₆alkoxycarbonyl, C₁₋₆alkylsulfonyl, aminosulfonyl, C₁₋₆alkylaminosulfonyl, diC₁₋₆alkylaminosulfonyl, 3- to 8-membered cycloalkyl, 3- to 8-membered cycloalkylC₁₋₆alkyl, 3- to 8-membered cycloalkylC₁₋₆alkoxy, 4- to 8-membered heterocycloalkylC₁₋₆alkyl, 4- to 8-membered heterocycloalkylC₁₋₆alkoxy or 4- to 8-membered heterocycloalkyl containing N atom in its ring structure.

In Reaction Scheme 2, the compound of formula 1a substituted with an amine compound may be prepared by reacting a compound of formula 14 with a compound of P²—R⁶R⁷ having various leaving groups according to conventional substitution methods. The compound of formula 14 may be prepared by nitrating a hetero ring compound of formula 9 using KNO₃ and sulfuric acid according to a conventional nitration method to obtain a compound of formula 10, i.e., a compound of formula 9 in which the 6-position is substituted with nitro group; reacting the compound of formula 10 with a piperazine derivative of formula 11 to obtain a compound of formula 12; reducing the compound of formula 12 to quantitatively obtain an amine compound of formula 13; and reacting the compound of formula 13 with the oxirane compound of formula 6 to stereoselectively obtain a compound of formula 14 having opened epoxide ring. Further, the compound of formula 14 may be prepared by reacting the compound of formula 12 with the compound of formula 6 to obtain a compound of formula 15; and reducing the compound of formula 15.

The compound of formula 1b, i.e., a compound of formula 1 wherein A is a direct bond or CH₂ and R is

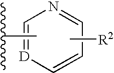

may be prepared by a method shown in Reaction Scheme 3.

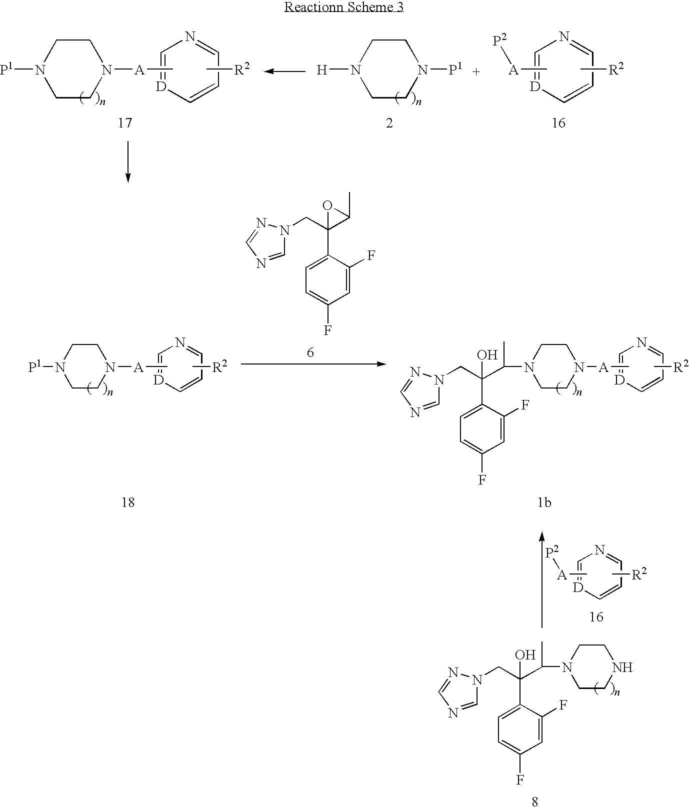

wherein, n, D, P¹, P² and R² have the same meanings as defined above.

In Reaction Scheme 3, similarly to the method described in Reaction Scheme 1, the compound of formula 1b may be prepared by reacting a heteroaryl compound of formula 16 having leaving group P² with the piperazinyl compound of formula 2 to obtain a heteroaryl compound of formula 17 substituted with piperazinyl, removing the amine protecting group from the compound of formula 17 to obtain a compound of formula 18 (except that P¹ is hydrogen), reacting the compound of formula 18 with the oxirane compound of formula 6 as described in Reaction Scheme 1. Further, in Reaction Scheme 3, similarly to the method described in Reaction Scheme 1, the compound of formula 1b may be prepared by allowing the direct reaction of the compound of formula 8 with the compound of formula 15.

The reaction of the compound of formula 16 with the compound of formula 2 may be conducted at a temperature ranging from 100 to 180° C. when the leaving group is linked to carbon atom adjacently to nitrogen atom, or may be carried out through amination using a palladium catalyst according to conventional methods described in Buchwald, S. L. et al., J. Org. Chem. 60 (2000), 1158; and Heo, J.-N. et al., Tetrahedron Letters, 46 (2005), 4621.

The compounds of formulae 1c-1 (Z═O) and 1c-2 (Z═S), i.e., a compound of formula 1 wherein A is C═O and R is

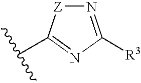

may be prepared by methods described in Reaction Schemes 4 and 5, respectively.

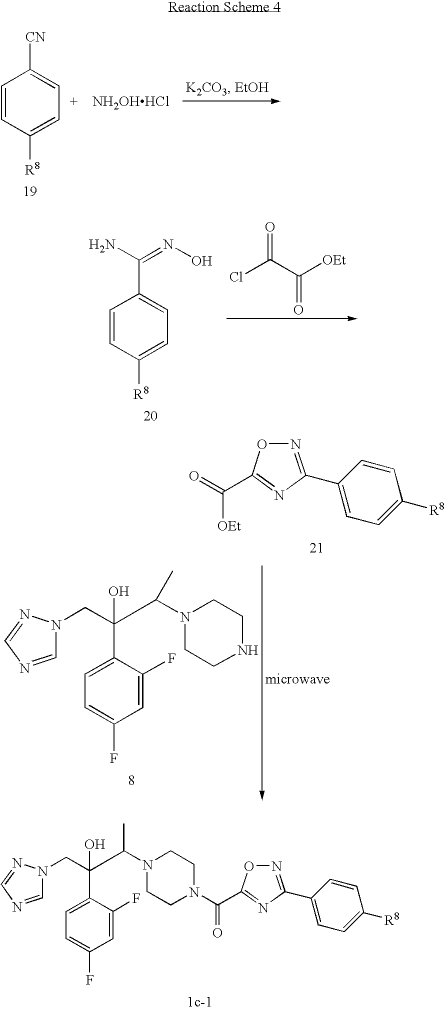

wherein,

R⁸ is hydrogen, halogen, hydroxy, C₁₋₆alkoxy cyano, nitro, amino, hydroxycarbonyl, C₁₋₆alkyl, C₁ alkenyl, C₁₋₆alkynyl, C₁₋₆alkoxy, hydroxyC₁₋₆alkyl, C₁₋₆alkoxyC₁₋₆alkyl, perfluoroC₁₋₆alkyl, perfluoroC₁₋₆alkoxy, C₁₋₆alkylamino, diC₁₋₆alkylamino, aminoC₁₋₆alkyl, C₁₋₆alkylaminoC₁₋₆alkyl, diC₁₋₆alkylaminoC₁₋₆alkyl, C₁₋₆acyl, C₁₋₆acyloxy, C₁₋₆acyloxyC₁₋₆alkyl, C₁₋₆acylamino, C₁₋₆alkylthio, C₁₋₆alkylthiocarbonyl, C₁₋₆ alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl or 4- to 8-membered heterocycloalkyl; and n has the same meaning as defined above.

In Reaction Scheme 4, the compound of formula 1c-1 may be prepared by reacting a 5-aryl-1,2,4-oxadiazol-3-carboxylate derivative of formula 21 with the compound of formula 8 using microreactor. The compound of formula 21 may be prepared by reacting a compound of formula 19 with hydroxylamine in the presence of a base, e.g., potassium carbonate, sodium carbonate and sodium hydrogen carbonate, to obtain a hydroxybenzimideamide derivative of formula 20, and reacting the compound of formula 20 with ethyl chlorooxoacetate, according to conventional methods described in Goncalves, H et al., Bull. Soc. Chim. Fr. (1970), 7, 2589 and Berndt, E. W et al., J. Heterocyclic Chem. (1972) 9, 137.

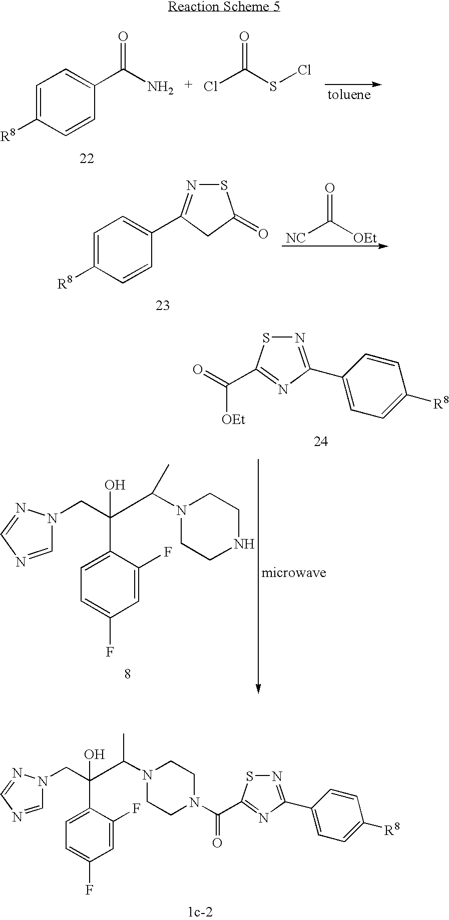

wherein, n and R⁸ have the same meanings as defined above.

In Reaction Scheme 5, the compound of formula 1c-2 may be prepared by reacting a 1,2,4-thiadiazole derivative of formula 24 with the compound of formula 8 using a microreactor. The compound of formula 24 may be prepared by reacting a benzamide compound of formula 22 with chlorocarbonylsulfenylchloride to obtain a 1,2,4-oxathiazol-5-one compound of formula 23, and reacting the compound of formula 23 with ethylcyanoformate, according to a conventional methods described in Howe, R. K. et al., J. Org. Chem. (1974), 39(7), 962-4.

The compound of formula 1d, a compound of formula 1 wherein A is C═O and R is

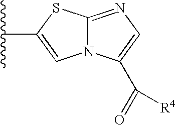

may be prepared by a method shown in Reaction Scheme 6.

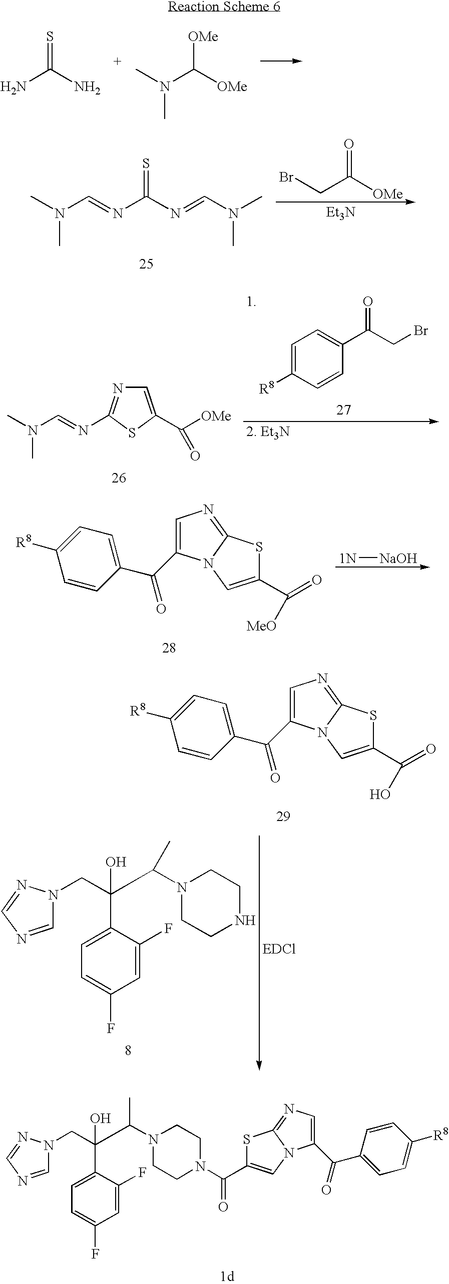

wherein, n and R⁸ have the same meanings as defined above.

In Reaction Scheme 6, the compound of formula 1d may be prepared by hydrolizing a compound of formula 2 synthesized by a conventional method described in Landreau, C. et al., J. Org. Chem. (2003), 68(12), 4912-4917 in the presence of a base, e.g., sodium hydroxide, to obtain a carboxylic acid compound of formula 29, and reacting the compound of formula 29 with the piperazinyl compound of formula 8 using a conventional peptide coupling reagent.

The pharmaceutically acceptable salts, hydrates, solvates or isomers of the compound of formula 1 may be derived from the compound of formula 1 according to conventional methods.

The inventive triazole compound of formula 1 and the pharmaceutically acceptable salt or isomer have a high antifungal activity against variable pathogenic fungi. Representative examples of the pathogenic fungi may include Candida, Cryptococcus, Aspergillus, Mucor, Histoplasma, Blastomyces, Coccidioides, Paracoccidioides, Trichophyton, Epidermophyton, Microsporum, Malassezia, Pseudallescheria, Sporothrix, Phinosporidium, Alternaria, Aureobasidium, Chaetomium and Curvularia.

Further, the present invention includes within its scope a pharmaceutical composition for treating diseases caused by fungal infection comprising the triazole derivative of formula 1 or the pharmaceutically acceptable salt, hydrate, solvate or isomer as an active ingredient.

The inventive pharmaceutical composition may be formulated for oral or perentaral administration, in a conventional manner together with one or more pharmaceutically acceptable excipients, binding agents, lubricants, disintergents, emulsifying agents, suspending agents, solvents, stabilizing agents, wet strength agents and ointments. For oral administration, the pharmaceutical composition of the present invention may take the form of tablet, coated tablet, powder, rigid or soft gelatin capsule, solution, dispersion, emulsion, syrup or granule prepared in the conventional manner together with diluents (e.g., lactose, dextrose, sucrose, mannitol, sorbitol, cellulose and/or glycine); and lubricants (e.g., silica, talc, stearic acid, magnesium or calcium stearate, and/or polyethyleneglycol). Further, the inventive tablet may comprise binding agents such as magnesium aluminum silicate, starch paste, gelatin, tragacanth, methylcellulose, sodium carboxymethylcellulose and/or polyvinylpyrrolidone, and may further comprise disintergents such as starch, agar, alginic acid and sodium alginate; effervescent mixtures; absorbing agents; coloring agents; flavoring agents; and sweetening agents, if necessary.

Further, the inventive composition for parenteral administration may take the form of sterilized aqueous solution, nonaqueous solution, suspension, emulsion, freeze-dried formulation or suppository. The nonaqueous solution and suspension of the present invention may be formulated using vegetable oils such as propyleneglycol, polyethyleneglycol and olive oil, or injectable esters such as ethyloleate; and the suppository may be formulated using witepsol, macrogol, Tween 61, cacao butter, laurin fat, glycerol or gelatin. Further, in the case of local or transdermal administration, the composition may be formulated in various forms such as ointment, cream, gel or solution, and the composition for intravenous injection may be an injective solution form.

The pharmaceutical composition may be sterilized or/and may further comprise antiseptics, stabilizing agents, wetting agents, emulsifying agents, supplements including salts and/or buffers for osmoregulation, and other therapeutically available materials, and may be formulated according to conventional mixing, granulating or coating methods.

Further, a proposed daily dose of the compound of the present invention for oral administration to an adult of 70 kg body weight is about from 1 mg to 2000 mg, more preferably about from 5 mg to 1000 mg; and for intravenous administration, about from 0.1 mg to 600 mg, more preferably about from 0.5 mg to 500 mg. It should be understood that the daily dose should be determined in light of various relevant factors including the condition to be treated, the severity of the patient's symptoms, the route of administration, or the physiological form of the anticancer agent; and, therefore, the dosage suggested above should not be construed to limit the scope of the invention in anyway.

The following Examples are intended to further illustrate the present invention without limiting its scope.

### EXAMPLE 1

### Preparation of (2R,3R)-3-(4-(benzooxazol-2-yl)piperazin-1-yl)-2-(2,4-difluorophenyl)-1-(1H-1,2,4-triazol-1-yl)butan-2-ol

Step 1

0.90 g (10.4 mmol) of piperazine was dissolved in 50 ml of dichloromethane in a dried round flask provided with nitrogen gas, 0.80 g (5.2 mmol) of 2-chlorobenzooxazole and 0.9 ml (52.1 mmol) of triethylamine were added thereto at 0° C., and the mixture was incubated at 0° C. for 30 mins. After adding water thereto, the reaction mixture was extracted with ethyl acetate, and the formed organic layer was dried over anhydrous magnesium sulfate and concentrated under a reduced pressure. The resulting residue was subjected to silica gel column chromatography (dichloromethane:methanol=9:1) to obtain 0.58 g of 2-(piperazin-1-yl)benzooxazole (yield: 55%).

¹H NMR (300 MHz, CDCl₃) δ 7.37 (d, 1H, J=7.5 Hz), 7.26 (d, 1H, J=7.5 Hz), 7.17 (t, 1H, J=7.7 Hz), 7.03 (t, 1H, J=7.7 Hz), 3.72 (t, 4H, J=4.8 Hz), 3.03 (t, 4H, J=4.9 Hz), 2.69 (s, 1H).

Step 2

0.60 g (2.4 mmol) of an oxirane compound was dissolved in acetonitrile in a dried round flask provided with nitrogen gas, 0.50 g (2.4 mmol) of the compound obtained in Step 1 and 0.39 g (3.7 mmol) of lithium perchlorate were added thereto, and the mixture was refluxed with stirring for 24 hrs. After adding distilled water and ethyl acetate thereto, the aqueous layer was further extracted with an organic solvent (more than 3 times), and the formed organic layer was washed with a saturated NaCl solution, dried over anhydrous magnesium sulfate and filtered. The resulting solution was distilled under a reduced pressure, and the residue was subjected to silica gel column chromatography (dichloromethane:methanol=49:1) to obtain 0.54 g of the title compound (yield: 50%).

¹H NMR (200 MHz, CDCl₃) δ 7.92 (s, 1H), 7.79 (s, 1H), 7.62-7.25 (m, 4H), 7.07 (dt, 1H, J=1.1, 7.5 Hz), 6.81-6.67 (m, 2H), 5.06 (br s, 1H), 5.01-4.85 (m, 2H), 3.66 (br s, 4H), 3.14-3.05 (m, 3H), 2.65-2.54 (m, 2H), 0.92 (d, 3H, J=7.0 Hz).

### EXAMPLE 2

### Preparation of (2R,3R)-3-(4-(benzothiazol-2-yl)piperazin-1-yl)-2-(2,4-difluorophenyl)-1-(1H-1,2,4-triazol-1-yl)butan-2-ol

Step 1

0.50 g (5.90 mmol) of piperazine was dissolved in 10 ml of 80% methanol/water in a dried round flask provided with nitrogen gas, 1.0 g (11.9 mmol) of sodium hydrogen carbonate and 0.50 g (3.0 mmol, 1 eq) of 2-chlorobenzothiazole was slowly added thereto, and the mixture was refluxed with stirring and heating for 12 hrs. After adding water thereto, the reaction mixture was extracted with ethyl acetate, and the formed organic layer was dried over anhydrous magnesium sulfate and concentrated under a reduced pressure. The resulting residue was subjected to silica gel column chromatography (dichloromethane:methanol=9:1) to obtain 0.61 g of 2-(piperazin-1-yl)benzothiazole (yield: 94%).

¹H NMR (300 MHz, CDCl₃) δ 7.62-7.54 (m, 2H), 7.30 (t, 1H, J=7.7 Hz), 7.08 (t, 1H, J=7.6 Hz), 3.65 (t, 4H, J=5.1 Hz), 3.04 (t, 4H, J=5.1 Hz), 2.55 (br s, 1H); MS (EI) m/z C₁₁H₁₃N₃S calc. 219, found 219 (M⁺, 13), 135 (32), 42 (100).

Step 2

The procedure of Step 2 of Example 1 was repeated except for using the compound obtained in Step 1 instead of 2-(piperazin-1-yl)benzooxazole to obtain 0.30 g of the title compound (yield: 27%).

¹H NMR (200 MHz, CDCl₃) δ 7.92 (s, 1H), 7.79 (s, 1H), 7.62-7.25 (m, 4H), 7.07 (dt, 1H, J=1.1, 7.5 Hz), 6.81-6.67 (m, 2H), 5.06 (br s, 1H), 5.01-4.85 (m, 2H), 3.66 (br s, 4H), 3.14-3.05 (m, 3H), 2.65-2.54 (m, 2H), 0.92 (d, 3H, J=7.0 Hz); MS (EI) m/z C₂₃H₂₄F₂N₆OS calc. 470, found 469 (M⁺−1, 1), 388 (1), 246 (100).

### EXAMPLE 3

### Preparation of (2R,3R)-2-(2,4-difluorophenyl)-3-(4-(1-methyl-1H-benzoimidazol-2-yl)piperazin-1-yl)-1-(1H-1,2,4-triazol-1-yl)butan-2-ol

Step 1

1.5 g (9.8 mmol, 1 eq) of 2-chlorobenzoimidazole was dissolved in 10 ml of DMF in a dried round flask provided with nitrogen gas, 0.47 g (11.8 mmol, 1.2 eq) of sodium hydride was slowly added thereto at 0° C., and the mixture was incubated at room temperature for 1 hr. 1.7 g (11.8 mmol, 1.2 eq) of iodomethane was added thereto, followed by allowing the reaction mixture to react for 1 hr. After adding cold water thereto, the resulting solid was filtered, washed with ethyl acetate, and dried to obtain 1.3 g of 2-chloro-1-methylbenzoimidazole (yield: 79%).

¹H NMR (200 MHz, CDCl₃) δ 7.77-7.66 (m, 1H), 7.30-7.27 (m, 3H), 3.78 (s, 3H); MS (EI) m/z C₈H₇ClN₂ calc. 166, found 166 (M⁺, 4), 43 (100).

Step 2

0.43 g (5.0 mmol) of piperazine and 0.24 g (1.0 mmol) of the compound obtained in Step 1 were placed in a dried round flask provided with nitrogen gas, followed by reacting the mixture at 150° C. for 30 mins. After cooling to room temperature, the reaction mixture was acidified with 1N—HCl solution and washed with dichloromethane. The water layer was treated with 1N—NaOH solution and extracted with dichloromethane. The formed organic layer was dried over anhydrous magnesium sulfate and concentrated under a reduced pressure, and the resulting residue was subjected to silica gel column chromatography (dichloromethane:methanol=9:1) to obtain 0.15 g of 1-methyl-2-(piperazin-1-yl)-benzoimidazole (yield: 69%).

¹H NMR (300 MHz, CDCl₃) δ 7.62-7.59 (m, 1H), 7.20-7.16 (m, 3H), 3.61 (s, 3H), 3.30-3.27 (m, 4H), 3.10-3.06 (m, 4H), 2.03 (br s, 1H); MS (EI) m/z C₁₂H₁₆N₄ calc. 216, found 216 (M⁺, 7), 160 (100), 131 (20).

Step 3

The procedure of Step 2 of Example 1 was repeated except for using the compound obtained in Step 2 instead of 2-(piperazin-1-yl)benzooxazole to obtain the title compound (yield: 43%).

¹H NMR (200 MHz, CDCl₃) δ 7.97 (s, 1H), 7.79 (s, 1H), 7.63-7.41 (m, 2H), 7.20-7.16 (m, 3H), 6.81-6.70 (m, 2H), 5.05 (s, 1H), 4.96 (d, 1H, J=14.4 Hz), 4.86 (d, 1H, J=14.6 Hz), 3.61 (s, 3H), 3.42-3.33 (m, 4H), 3.09-2.98 (m, 3H), 2.70-2.62 (m, 2H), 1.01 (d, 3H, J=6.8 Hz); MS (EI) m/z C₂₄H₂₇F₂N₇O calc. 467, found 468 (M⁺+1, 1), 385 (2), 243 (100).

### EXAMPLE 4

### Preparation of (2R,3R)-3-(4-(6-chlorobenzothiazol-2-yl)piperazin-1-yl)-2-(2,4-difluorophenyl)-1-(1H-1,2,4-triazol-1-yl)butan-2-ol

Step 1

The procedure of Step 1 of Example 2 was repeated except for using 2,6-dichlorobenzothiazole instead of 2-chlorobenzothiazole and conducting the reaction for 18 hrs to obtain 6-chloro-2-(piperazin-1-yl)benzothiazole (yield: 89%).

¹H NMR (300 MHz, CDCl₃) δ 7.56 (d, 1H, J=2.1 Hz), 7.44 (d, 1H, J=8.7 Hz), 7.24 (dd, 1H, J=2.1, 8.4 Hz), 3.62-3.58 (m, 4H), 3.02-2.99 (m, 4H); MS (ESI) m/z C₁₁H₁₂ClN₃S calc. 253, found 253.39.

Step 2

The procedure of Step 2 of Example 1 was repeated except for using the compound obtained in Step 1 instead of 2-(piperazin-1-yl)benzooxazole and propionitrile instead of acetonitrile to obtain the title compound (yield: 65%).

¹H NMR (300 MHz, CDCl₃) δ 7.92 (s, 1H), 7.79 (s, 1H), 7.57 (d, 1H, J=2.1 Hz), 7.48-7.40 (m, 2H), 7.26-7.23 (m, 1H), 6.80-6.69 (m, 2H), 5.02 (s, 1H), 4.97 (d, 1H, J=14.4 Hz) 4.91 (d, 1H, J=15.3 Hz), 3.65 (br s, 4H), 3.11-3.04 (m, 3H), 2.64-2.57 (m, 2H), 0.92 (d, 3H, J=6.6 Hz); MS (ESI) m/z C₂₃H₂₃ClF₂N₆OS calc. 504.13, found 504.28.

### EXAMPLE 5

### Preparation of (2R,3R)-3-(4-(5-chlorobenzo oxazol-2-yl)piperazin-1-yl)-2-(2,4-difluorophenyl)-1-(1H-1,2,4-triazol-1-yl)butan-2-ol

Step 1

2-amino-4-chlorophenol 1.0 g (7 mmol) and 1.34 g (8.4 mmol) of potassium O-ethylxanthate were dissolved in 20 ml of ethanol in a dried round flask provided with nitrogen gas, followed refluxing the mixture with stirring for 16 hrs. The resulting solution was concentrated under a reduced pressure, mixed with ethylacetate, and washed with water. The formed organic layer was washed with a sodium hydrogen carbonate solution and a saturated NaCl solution, dried over anhydrous magnesium sulfate, and distilled under a reduced pressure. The resulting residue was subjected to silica gel column chromatography (dichloromethane:methanol=49:1) to obtain 5-chloro-2-thiobenzooxazole (yield: 57%).

¹H NMR (300 MHz, CD₃OD) δ 7.27-7.13 (m, 3H); MS (ESI) m/z C₇H₄ClNOS calc. 184.97, found 185.98 (M⁺+1).

Step 2

2.97 g (15.9 mmol) of the compound obtained in Step 1 and 4.43 g (23.8 mmol) of 1-tert-butyloxycarbonylpiperazine were placed in a 250 mL flask, p-xylene was added thereto, and the mixture was incubated at 138° C. for 15 hrs. The reaction mixture was concentrated under a reduced pressure, dissolved in ethylacetate, and washed with water. The formed organic layer was washed with a sodium hydrogen carbonate solution and a saturated NaCl solution, dried over anhydrous magnesium sulfate, filtered, and distilled under a reduced pressure. The resulting residue was subjected to silica gel column chromatography (n-hexane:ethyl acetate=9:1) to obtain tert-butyl 4-(5-chlorobenzooxazol-2-yl)piperazine-1-carboxylate (yield: 63%).

¹H NMR (CDCl₃) δ 7.32 (d, J=2 Hz, 1H), 7.18-7.14 (d, J=8.4 Hz, 1H), 7.02-6.97 (dd, J=8.6, 2.0 Hz, 1H), 3.70-3.65 (m, 4H), 3.59-3.54 (m, 4H), 1.49 (s, 9H); MS (ESI) m/z C₁₆H₂₀ClN₃O₃ calc. 337.12, found 338.17 (M⁺+1).

Step 3

200 mg (0.6 mmol) of the compound obtained in Step 2 was dissolved in 5 ml of dichloromethane in a dried round flask provided with nitrogen gas, followed by slowly adding dropwise 410 μl of trifluoroacetic acid thereto. The mixture was kept at room temperature for 4 hrs, concentrated under a reduced pressure, dissolved in ethylacetate, and washed with water. The formed organic layer was washed with a sodium hydrogen carbonate solution and a saturated NaCl solution, dried over anhydrous magnesium sulfate, filtered, and distilled under a reduced pressure to quantitatively obtain 5-chloro-2-(piperazin-1-yl)benzooxazole.

Step 4

The procedure of Step 2 of Example 1 was repeated except for using the compound obtained in Step 3 instead of 2-(piperazin-1-yl)benzooxazole and propionitrile instead of acetonitrile to obtain the title compound (yield: 60%).

¹H NMR (200 MHz, CDCl₃) δ 7.90 (s, 1H), 7.79 (s, 1H), 7.50-7.37 (m, 1H), 7.31-7.27 (m, 1H), 7.18-7.12 (m, 1H) 7.02-6.95 (m, 1H), 6.81-6.67 (m, 2H), 5.00-4.86 (m, 3H), 3.73 (s, 4H), 3.13-3.06 (m, 3H), 2.64-2.53 (m, 2H), 0.91 (d, J=6.2 Hz, 3H); MS (ESI) m/z C₂₃H₂₃ClF₂N₆O₂ cacl. 488.15, found 489.26 (M⁺+1).

### EXAMPLE 6

### Preparation of (2R,3R)-3-(4-(6-chlorobenzooxazol-2-yl)piperazin-1-yl)-2-(2,4-difluorophenyl)-1-(1H-1,2,4-triazol-1-yl)butan-2-ol

Step 1

The procedure of Step 1 of Example 5 was repeated except for using 2-amino-5-chlorophenol instead of 2-amino-4-chlorophenol to obtain 6-chloro-2-thiobenzooxazole (yield: 78%).

MS (ESI) m/z C₇H₄ClNOS cacl. 184.97, found 185.98 (M⁺+1).

Step 2

The procedure of Step 2 of Example 5 was repeated except for using the compound obtained in Step 1 instead of 5-chloro-2-thiobenzooxazole to obtain 4-(6-chlorobenzooxazol-2-yl)piperazine-1-carboxylate (yield: 50%).

¹H NMR (200 MHz, CDCl₃) δ 7.26 (s, 1H), 7.22 (m, 1H), 7.17-7.16 (m, 1H), 3.69-3.63 (m, 4H), 3.58-3.53 (m, 4H), 1.49 (s, 9H); MS (ESI) m/z C₁₆H₂₀ClN₃O₃ cacl. 337.12, found 338.15 (M⁺+1).

Step 3

The procedure of Step 3 of Example 5 was repeated except for using the compound obtained in Step 2 instead of 4-(5-chlorobenzooxazol-2-yl)piperazine-1-carboxylate to quantitatively obtain 6-chloro-2-(piperazin-1-yl)benzooxazole.

Step 4

The procedure of Step 2 of Example 1 was repeated except for using the compound obtained in Step 3 instead of 2-(piperazin-1-yl)benzooxazole to obtain the title compound (yield: 58%).

¹H NMR (300 MHz, CDCl₃) δ 7.90 (s, 1H), 7.79 (s, 1H), 7.42 (dd, 1H, J=6.5, 8.8 Hz), 7.26-7.22 (m, 2H), 7.16-7.12 (m, 1H), 6.80-6.69 (m, 2H), 5.00-4.88 (m, 3H), 3.72 (br s, 4H), 3.09-3.04 (m, 3H), 2.63-2.56 (m, 2H), 0.91 (d, 3H, J=6.9 Hz); MS (EI) m/z C₂₃H₂₃ClF₂N₆O₂ calc. 488, found 488 (M⁺, 2), 406 (11), 264 (100).

### EXAMPLE 7

### Preparation of (2R,3R)-2-(2,4-difluorophenyl)-3-(4-(6-fluorobenzothiazol-2-yl)piperazin-1-yl)-1-(1H-1,2,4-triazol-1-yl)butan-2-ol

Step 1

The procedure of Step 1 of Example 2 was repeated except for using 2-chloro-6-fluorobenzothiazole instead of 2-chlorobenzothiazole to obtain 6-fluoro-2-(piperazin-1-yl)-benzothiazole (yield: 85%).

¹H NMR (300 MHz, CDCl₃) δ 7.49-7.44 (m, 1H), 7.31 (dd, 1H, J=2.6, 8.2 Hz), 7.02 (dt, 1H, J=2.6, 9.0 Hz), 3.61-3.58 (m, 4H), 3.03-2.99 (m, 4H); MS (EI) m/z C₁₁H₁₂FN₃S, calc. 237.07, found 237.07 (100), 207.0 (9), 195.0 (81), 180.9 (34).

Step 2

The procedure of Step 2 of Example 1 was repeated except for using the compound obtained in Step 1 instead of 2-(piperazin-1-yl)benzooxazole to obtain the title compound (yield: 60%).

¹H NMR (300 MHz, CDCl₃) δ 7.91 (s, 1H), 7.79 (s, 1H), 7.49-7.40 (m, 2H), 7.33-7.29 (m, 1H), 7.02 (dt, 1H, J=2.6, 9.0 Hz), 6.80-6.69 (m, 2H), 5.02 (s, 1H), 4.99-4.87 (m, 2H), 3.63 (br s, 4H), 3.11-3.04 (m, 3H), 2.63-2.56 (m, 2H), 0.92 (d, 3H, J=6.7 Hz); MS (EI) m/z C₂₃H₂₃F₃N₆OS, cald. 488.16, found 489 (M⁺, 1), 264 (100).

### EXAMPLE 8

### Preparation of (2R,3R)-2-(2,4-difluorophenyl)-3-(4-(6-methylbenzothiazol-2-yl)piperazin-1-yl)-1-(1H-1,2,4-triazol-1-yl)butan-2-ol

Step 1

The procedure of Step 1 of Example 2 was repeated except for using 2-chloro-6-methylbenzothiazole instead of 2-chlorobenzothiazole to obtain 6-methyl-2-(piperazin-1-yl)-benzothiazole (yield: 86%).

¹H NMR (300 MHz, CDCl₃) δ 7.46-7.41 (m, 2H), 7.12-7.09 (m, 1H), 3.61-3.58 (m, 4H), 3.02-2.99 (m, 4H), 2.39 (s, 3H); MS (EI) m/z C₁₂H₁₅N₃S, calc. 233.1, found 233 (100), 191 (93), 177 (83), 165 (41), 150 (13).

Step 2

The procedure of Step 2 of Example 1 was repeated except for using the compound obtained in Step 1 instead of 2-(piperazin-1-yl)-benzooxazole to obtain the title compound (yield: 86%).

¹H NMR (300 MHz, CDCl₃) δ 7.92 (s, 1H), 7.79 (s, 1H), 7.49-7.41 (m, 3H), 7.10 (dd, 1H, J=1.2, 8.2 Hz), 6.80-6.69 (m, 2H), 5.04 (s, 1H), 4.97 (d, 1H, J=14.8 Hz), 4.89 (d, 1H, J=15.2 Hz), 3.63 (br s, 4H), 3.09-3.02 (m, 3H), 2.62-2.55 (m, 2H), 2.05 (s, 3H), 0.93 (d, 3H, J=6.9 Hz); MS (EI) m/z C₂₄H₂₆F₂N₆OS, calc. 484.19, found 484 (1), 260 (100), 191 (8).

### EXAMPLE 9

### Preparation of (2R,3R)-2-(2,4-difluorophenyl)-3-(4-(6-methoxybenzothiazol-2-yl)piperazin-1-yl)-1-(1H-1,2,4-triazol-1-yl)butan-2-ol

Step 1

The procedure of Step 1 of Example 2 was repeated except for using 2-chloro-6-methoxybenzothiazole instead of 2-chlorobenzothiazole to obtain 6-methoxy-2-(piperazin-1-yl)-benzothiazole (yield: 79%).

¹H NMR (300 MHz, CDCl₃) δ 7.46 (d, 1H, J=8.8 Hz), 7.15 (d, 1H, J=2.6 Hz), 6.90 (dd, 1H, J=2.6, 8.8 Hz), 3.81 (s, 3H), 3.59-3.56 (m, 4H), 3.02-2.99 (m, 4H); MS (EI) m/z C₁₂H₁₅N₃OS, calc. 249.09, found 249 (100), 207 (18), 193 (13), 180 (11), 166 (9).

Step 2

The procedure of Step 2 of Example 1 was repeated except for using the compound obtained in Step 1 instead of 2-(piperazin-1-yl)benzooxazole to obtain the title compound (yield: 95%).

¹H NMR (300 MHz, CDCl₃) δ 7.92 (s, 1H), 7.79 (s, 1H), 7.48-7.41 (m, 2H), 7.15 (d, 1H, J=2.6 Hz), 6.90 (dd, 1H, J=2.6, 8.8 Hz), 6.80-6.69 (m, 2H), 5.05 (s, 1H), 4.96 (d, 1H, J=14.7 Hz), 4.89 (d, 1H, J=15.7 Hz), 3.82 (s, 3H), 3.61 (br s, 4H), 3.09-3.04 (m, 3H), 2.62-2.55 (m, 2H), 0.93 (d, 3H, J=6.9 Hz); MS (EI) m/z C₂₄H₂₆F₂N₆O₂S, calc. 500.18, found 500 (1), 276 (100), 233 (11), 207 (17).

### EXAMPLE 10

### Preparation of (2R,3R)-2-(2,4-difluorophenyl)-3-(4-(6-(piperidin-1-yl)benzothiazol-2-yl)piperazin-1-yl)-1-(1H-1,2,4-triazol-1-yl)butan-2-ol

Step 1

The procedure of Step 1 of Example 2 was repeated except for using 2,6-dichlorobenzothiazole instead of 2-chlorobenzothiazole and conducting the reaction for 18 hrs to obtain tert-butyl 4-(6-chlorobenzothiazol-2-yl)piperazine-1-carboxylate (yield: 73%).

¹H NMR (300 MHz, CDCl₃) δ 7.57 (d, J=2.0 Hz, 1H), 7.45 (d, J=8.8 Hz, 1H), 7.28-7.23 (m, 1H), 3.59 (s, 8H), 1.49 (s, 9H); MS (ESI) m/z C₁₆H₂₀ClN₃O₂S calc. 353.10, found 353.31.

Step 2

100 mg (0.28 mmol) of the compound obtained in Step 1, 29 mg (0.34 mmol) of piperidine, 5 mg (0.0056 mmol) of tris(dibenzylideneacetone)dipalladium(0), 3.3 mg (0.0085 mmol) of 2-dicyclohexylphosphino-2′-(N,N-dimethylamino)biphenyl and 38 mg (0.40 mmol) of sodium t-butoxide were placed in 5 mL of a microwave reactor, followed adding 3 mL of toluene thereto. The mixture was kept at 150° C. for 10 mins, and filtered using a cellite. The resulting solution was distilled under a reduced pressure and subjected to silica gel column chromatography (n-hexane:ethyl acetate=9:1) to obtain tert-butyl 4-(6-(piperidin-1-yl)benzothiazol-2-yl)piperazine-1-carboxylate (yield: 73%).

¹H NMR (300 MHz, CDCl₃) δ 7.92 (s, 1H), 7.78 (s, 1H), 7.46-7.40 (m, 2H), 7.26-7.16 (m, 3H), 6.98 (dd, J=8.7, 2.1 Hz, 1H) 6.79-6.70 (m, 2H), 4.98-4.86 (m, 3H), 3.60 (s, 4H), 3.11-3.01 (m, 7H), 2.57 (m, 2H), 2.35 (s, 1H), 1.77-1.69 (m, 4H), 1.59-1.26 (m, 2H), 0.93 (d, J=6.9 Hz, 3H).

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain 2-(piperazin-1-yl)-6-(piperidin-1-yl)benzothiazole.

Step 4

The procedure of Step 2 of Example 1 was repeated except for using the compound obtained in Step 3 instead of 2-(piperazin-1-yl)benzooxazole and propionitrile instead of acetonitrile to obtain the title compound (yield: 52%).

¹H NMR (300 MHz, CDCl₃) δ 7.92 (s, 1H), 7.78 (s, 1H), 7.46-7.40 (m, 2H), 7.26-7.16 (m, 1H), 6.98 (dd, J=8.8, 2.2 Hz, 1H), 6.79-6.69 (m, 2H), 5.05 (br s, 1H), 4.95 (d, J=14.1 Hz, 1H), 4.88 (d, J=15.6 Hz, 1H), 3.60 (s, 4H), 3.11-3.01 (m, 7H), 2.61-2.54 (m, 2H), 1.77-1.69 (m, 4H), 1.59-1.54 (m, 2H), 0.93 (d, J=6.6 Hz, 3H); MS (ESI) m/z C₂₈H₃₃F₂N₇OS, calc. 553.24, found 554.19 (M⁺+1).

### EXAMPLE 11

### Preparation of (2R,3R)-3-(4-(6-aminobenzothiazol-2-yl)piperazin-1-yl)-2-(2,4-difluorophenyl)-1-(1H-1,2,4-triazol-1-yl)butan-2-ol

Step 1

1.088 g (6.42 mmol) of 2-chlorobenzothiazole and 8.56 ml of sulfuric acid were placed in a 50 mL flask, followed by keeping the mixture at 10-17° C. for 1.5 hrs. After observing that the color of the reaction mixture was white, the reaction mixture was cooled to 12° C., 714 mg (7.062 mmol) of potassium nitrate was added thereto, and the resulting mixture was stirred with maintaining the reaction temperature less than 18° C. for 1 hr. The resulting solution was heated to 25° C., stirred for 1.5 hrs, and slowly heated to 40° C. After confirming the completion of the reaction by TLC, the resulting mixture was cooled to room temperature and poured into ice water, and the resulting solid was filtered, washed with water (until pH is 7), and dried under a vacuum condition to obtain 2-chloro-6-nitrobenzothiazole (yield: 87%).

¹H NMR (300 MHz, CDCl₃) δ 8.75 (dd, 1H, J=0.37, 2.3 Hz), 8.39 (dd, 1H, J=2.3, 9.0 Hz), 8.08 (dd, 1H, J=0.40, 9.0 Hz).

Step 2

The procedure of Step 1 of Example 2 was repeated except for using the compound obtained in Step 1 instead of 2-chlorobenzothiazole to obtain 6-nitro-2-(piperazin-1-yl)benzothiazole (yield: 66%).

¹H NMR (300 MHz, CDCl₃) δ 8.51 (d, 1H, J=2.3 Hz), 8.20 (dd, 1H, J=2.4, 8.9 Hz), 7.50 (d, 1H, J=8.9 Hz), 3.72-3.68 (m, 4H), 3.05-3.01 (m, 4H); MS (EI) m/z C₁₁H₁₂N₄O₂S, cald. 264.07, found 264 (M⁺, 40), 222 (100), 209 (49), 196 (70), 176 (93), 162 (79).

Step 3

306 mg (1.20 mmol) of the compound obtained in Step 2 was dissolved in THF (5 mL), and Pd/C (31 mg, 10 wt %) was added thereto. H₂ gas was introduced therein, and the mixture was reacted at room temperature for 19 hours. The reaction mixture was filtered using a cellite to remove palladium, and distilled under a reduced pressure. The resulting residue was subjected to silica gel column chromatography (dichloromethane:methanol=9:1) to obtain 6-amino-2-(piperazin-1-yl)benzothiazole (yield: 60%).

¹H NMR (300 MHz, CDCl₃) δ 7.36 (d, 1H, J=8.5 Hz), 6.94 (d, 1H, J=2.4 Hz), 6.68 (dd, 1H, J=2.4, 8.5 Hz), 3.55-3.51 (m, 5H), 3.00-2.96 (m, 6H).

Step 4

The procedure of Step 2 of Example 1 was repeated except for using the compound obtained in Step 3 instead of 2-(piperazin-1-yl)benzooxazole to obtain the title compound (yield: 56%).

¹H NMR (200 MHz, CDCl₃) δ 7.93 (s, 1H), 7.79 (t, 1H, J=6.5 Hz), 7.44-7.35 (m, 2H), 6.96 (d, 1H, J=2.0 Hz), 6.79-6.67 (m, 3H), 5.05 (s, 1H), 4.98-4.86 (m, 2H), 3.52 (br s, 6H), 3.08-3.01 (m, 3H), 2.62-2.54 (m, 2H), 0.93 (d, 3H, J=6.9 Hz).

### EXAMPLE 12

### Preparation of (2R,3R)-3-(4-(6-(2-morpholinoethylamino)benzothiazol-2-yl)piperazin-1-yl)-2-(2,4-difluorophenyl)-1-(1H-1,2,4-triazol-1-yl)butan-2-ol

115 mg (0.237 mol) of the compound obtained in Step 4 of Example 11, 66.2 mg (0.356 mmol) of 4-(2-chloroethyl)morpholine and 98.3 mg (0.711 mmol) of potassium carbonate were added to acetonitrile, followed by refluxing the mixture with stirring at 80° C. for 48 hrs. After confirming the completion of the reaction by TLC, the solvent was removed from the reaction mixture, and the resulting mixture was dissolved in ethyl acetate and washed with water. The formed organic layer was washed with a saturated NaCl solution, dried over anhydrous magnesium sulfate, and concentrated under a reduced pressure. The resulting residue was dissolved in ethyl acetate, and the resulting solution was recrystallized with n-hexane to obtain 92 mg of the title compound (yield: 65%).

¹H NMR (300 MHz, CDCl₃) δ 7.93 (s, 1H), 7.78 (s, 1H), 7.49-7.38 (m, 2H), 7.24-7.13 (m, 1H), 6.80 (d, 1H, J=2.4 Hz), 6.76-6.65 (m, 2H), 5.05 (s, 1H), 4.94 (d, 1H, J=15.0 Hz), 4.90 (d, 1H, J=15.0 Hz), 4.24 (br s, 1H), 3.72 (t, 4H, J=4.5 Hz), 3.58 (br s, 4H), 3.19-3.01 (m, 5H), 2.66-2.46 (m, 8H), 0.93 (d, 3H, J=6.9 Hz); MS (EI) m/z C₂₉H₃₆F₂N₃O₂S, calc. 598.26, found 598 (4), 374 (100), 339 (34), 325 (16), 311 (64).

### EXAMPLE 13

### Preparation of (2R,3R)-3-(4-(6-(N-methyl-N-(2-morpholinoethyl)amino)benzothiazol-2-yl)piperazin-1-yl)-2-(2,4-difluorophenyl)-1-(1H-1,2,4-triazol-1-yl)butan-2-ol

Step 1

The procedure of Step 2 of Example 10 was repeated except for using 2-morpholinoethaneamine instead of piperidine to obtain tert-butyl 4-(6-(2-morpholinoethylamino)benzothiazol-2-yl)piperazine-1-carboxylate (yield: 72%).

¹H NMR (300 MHz, CDCl₃) δ 7.40 (d, 1H, J=8.7 Hz), 6.89 (d, 1H, J=2.4 Hz), 6.68 (dd, 1H, J=2.1, 8.7 Hz), 4.27 (br s, 1H), 3.74-3.71 (m, 4H), 3.55 (br s, 8H), 3.17 (t, 2H, J=5.7 Hz), 2.65 (t, 2H, 4.2 Hz), 2.50-2.47 (m, 4H), 1.49 (s, 9H).

Step 2

150 mg (0.34 mmol) of the compound obtained in Step 1 and 10 mg (0.34 mmol) of formaldehyde were dissolved in dichloromethane in a dried round flask provided with nitrogen gas, sodium triacetoxyborohydride (0.47 mmol) was added thereto, and the mixture was kept at room temperature for 16 hrs. After adding water thereto, the reaction mixture was extracted with ethylacetate. The formed organic layer was washed with a saturated NaCl solution, dried over anhydrous magnesium sulfate, filtered, and distilled under a reduced pressure. The resulting residue was subjected to silica gel column chromatography to obtain tert-butyl 4-(6-(N-methyl-N-(2-morpholinoethyl)amino)benzothiazol-2-yl)piperazine-1-carboxylate.

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain N-methyl-N-(2-morpholinoethyl)-2-(piperazin-1-yl)benzothiazole-6-amine.

Step 4

The procedure of Step 2 of Example 1 was repeated except for using the compound obtained in Step 3 instead of 2-(piperazin-1-yl)-benzooxazole and propionitrile instead of acetonitrile to obtain the title compound (yield: 58%).

¹H NMR (300 MHz, CDCl₃) δ 7.93 (s, 1H), 7.90 (s, 1H), 7.46-7.41 (m, 2H), 6.97 (d, J=2.1 Hz, 1H), 6.80-6.69 (m, 3H), 5.05 (br s, 1H), 4.96 (d, J=14.4 Hz, 1H), 4.88 (d, J=15.0 Hz, 1H), 3.71 (t, J=4.5 Hz, 4H), 3.59 (s, 4H), 3.46 (t, J=7.2 Hz, 2H), 3.08-3.01 (m, 3H), 2.94 (s, 3H), 2.61-2.48 (m, 8H), 0.93 (d, J=6.6 Hz, 3H).

### EXAMPLE 14

### Preparation of (2R,3R)-3-(4-(6-nitro)benzothiazol-2-yl)piperazin-1-yl)-2-(2,4-difluorophenyl)-1-(1H-1,2,4-triazol-1-yl)butan-2-ol

Step 1

1.0 g (3.98 mmol) of an oxirane compound and 0.86 g (10 mmol) of piperazine were dissolved in 3 mL of acetonitrile in a dried 5 ml microwave reactor provided with nitrogen gas, lithium perchlorate 0.64 g (3.98 mmol) was added thereto, and the reactor was kept for 150° C. for 20 mins. After adding distilled water thereto, the reaction mixture was extracted 3 times with ethyl acetate. The formed organic layer was washed with a saturated NaCl solution, dried over anhydrous magnesium sulfate, and concentrated under a reduced pressure. The resulting residue was subjected to silica gel column chromatography (dichloromethane:methanol:triethylamine=9:1:0.5) to obtain (2R,3R)-2-(2,4-difluorophenyl)-3-(piperazin-1-yl)-1-(1H-1,2,4-triazol-1-yl)butan-2-ol (yield: 70%).

¹H NMR (200 MHz, CDCl₃) δ 7.99 (s, 1H), 7.78 (s, 1H), 7.54-7.27 (m, 1H), 6.82-6.67 (m, 2H), 5.3 (br s, 1H), 4.90 (d, 1H, J=14.6 Hz), 4.79 (d, 1H, J=15.0 Hz), 2.97-2.70 (m, 7H), 2.45-2.34 (m, 2H), 0.97 (d, 3H, J=7.2 Hz).

Step 2

168 mg (0.5 mmol) of the compound obtained in Step 1 and 107 mg (0.5 mmol) of the compound obtained in Example 11 were dissolved in 10 mL of acetonitrile, 207 mg (1.5 mmol) of potassium carbonate was added thereto, and the mixture was refluxed with stirring and heating for 16 hrs. After cooling to room temperature, the reaction mixture was filtered to remove the solid, and concentrated under a reduced pressure to remove the solvent. The resulting residue was dissolved in ethyl acetate and washed with water and a saturated NaCl solution, and the formed organic layer was dried over anhydrous magnesium sulfate, filtered and concentrated under a reduced pressure. The resulting residue was subjected to silica gel column chromatography (dichloromethane:methanol=19:1) to obtain 234 mg of the title compound (yield: 91%).

¹H NMR (300 MHz, CDCl₃) δ 8.51 (d, 1H, J=2.3 Hz), 8.20 (dd, 1H, J=9.0, 2.3 Hz), 7.90 (s, 1H), 7.80 (s, 1H), 7.51 (d, 1H, J=8.9 Hz), 7.47-7.39 (m, 1H), 6.80-6.69 (m, 2H), 5.01-4.95 (m, 3H), 3.75 (br s, 4H), 3.20-3.01 (m, 3H), 2.68-2.61 (m, 2H), 0.91 (d, 3H, J=6.8 Hz); MS (EI) m/z C₂₃H₂₃F₂N₇O₃S, calc. 515.16, found 516.1 (M⁺+1, 1), 291.0 (100), 261.1 (16), 222.0 (15), 140.9 (19).

### EXAMPLE 15

### Preparation of (2R,3R)-3-(4-(benzooxazol-2-yl)-1,4-diazepan-1-yl)-2-(2,4-difluorophenyl)-1-(1H-1,2,4-triazol-1-yl)butan-2-ol

Step 1

0.5 g (0.0025 mol) of tent-butyl 1,4-diazepane-1-carboxylate and 0.46 g (0.003 mol, 1.2 eq) of 2-benzooxazole were dissolved in 10 mL of chloroform in a dried round flask provided with nitrogen gas, followed by stirring the mixture at room temperature for 18 hrs. After diluting the reaction mixture with ethyl acetate, the water layer was extracted with ethyl acetate (more than 3 times), and the formed organic layer was washed with a saturated NaCl solution, dried over anhydrous magnesium sulfate, filtered and distilled under a reduced pressure. The resulting residue was subjected to column chromatography (ethyl acetate:n-hexane=9:1) to obtain tert-butyl 4-(benzooxazol-2-yl)-1,4-diazepane-1-carboxylate (yield: 50%).

¹H NMR (200 MHz, CDCl₃) δ 7.34-7.38 (m, 1H), 7.12-7.33 (m, 2H), 6.96-7.05 (m, 1H), 3.60-3.83 (m, 6H), 3.36-3.46 (m, 2H), 1.98-2.09 (m, 2H), 1.44 (s, 9H).

Step 2

The procedure of Step 3 of Example 5 was repeated except for using the compound obtained in Step 1 instead of tert-butyl 4-(5-chlorobenzooxazol-2-yl)piperazin-1-carboxylate to quantitatively obtain 2-(1,4-diazepan-1-yl)benzooxazole.

Step 3

The procedure of Step 2 of Example 1 was repeated except for using the compound obtained in Step 2 instead of 2-(piperazin-1-yl)benzooxazole and propionitrile instead of acetonitrile to obtain the title compound (yield: 42%).

¹H NMR (300 MHz, CDCl₃) δ 7.72 (s, 1H), 7.68 (s, 1H), 7.43-7.11 (m, 4H), 7.00-6.97 (m, 1H), 6.73-6.68 (m, 2H), 5.03 (br s, 1H), 4.74-4.69 (m, 2H), 3.90-3.73 (m, 4H), 3.23-3.07 (m, 3H), 2.80 (br s, 1H), 2.58 (br s, 1H), 2.01-1.98 (m, 2H), 0.91 (d, 3H, J=5.7 Hz); MS (EI) m/z C₂₄H₂₆F₂N₆O₂, calc. 468.21, found 466 (M⁺-2, 2), 386 (4), 244 (100), 141 (82).

### EXAMPLE 16

### Preparation of (2R,3R)-2-(2,4-difluorophenyl)-3-(4-(pyridin-2-yl)piperazin-1-yl)-1-(1H-1,2,4-triazol-1-yl)butan-2-ol

Step 1

0.1 g (0.63 mmol) of 2-bromopyridine and 0.065 g (0.75 mmol) of piperazine was reacted at 150° C. for 20 mins in a dried 5 mL microwave reactor provided with nitrogen gas. After cooling to room temperature, the reaction mixture was filtered using a cellite with in concurrence with washing with ethyl acetate, and distilled under a reduced pressure. The resulting residue was subjected to silica gel column chromatography (dichloromethane:methanol=4:1) to obtain 1-(pyridin-2-yl)piperazine (yield: 54%).

¹H NMR (200 MHz, CDCl₃) δ 8.20-8.18 (m, 1H), 7.52-7.43 (m, 1H), 6.66-6.59 (m, 2H), 3.53-3.34 (m, 4H), 3.01-2.96 (m, 4H), 2.1 (br s, 1H); MS (EI) m/z C₉H₁₃N₃ calc. 163.11, found 163 (M⁺, 29), 121 (63), 95 (100).

Step 2

The procedure of Step 2 of Example 1 was repeated except for using the compound obtained in Step 1 instead of 2-(piperazin-1-yl)benzooxazole to obtain the title compound (yield: 34%).

¹H NMR (200 MHz, CDCl₃) δ 8.18 (d, 1H, J=5.8 Hz), 7.79 (s, 1H), 7.55 (s, 1H), 7.43-7.55 (m, 2H), 6.59-5.81 (m, 4H), 5.21 (br s, 1H), 4.96 (d, 1H, J=14.6 Hz), 4.85 (d, 1H, J=14.6 Hz), 3.53 (br s, 4H), 2.88-3.01 (m, 3H), 2.48-2.59 (m, 2H), 0.96 (d, 3H, J=7.0 Hz).

### EXAMPLE 17

### Preparation of 5-(4-((2R,3R)-3-(2,4-difluorophenyl)-3-hydroxy-4-(1H-1,2,4-triazol-1-yl)butan-2-yl)piperazin-1-yl)picolinonitrile

Step 1

0.10 g (0.55 mmol) of 5-bromopicolinonitrile and 0.060 g (0.66 mmol) of piperazine were dissolved in DMF in a dried 5 ml microwave reactor provided with nitrogen gas, 0.090 g (0.66 mmol) of potassium carbonate was added thereto, and the reactor was kept at 200° C. for 30 mins. After cooling to room temperature, the reaction mixture was filtered using a cellite in concurrence with washing with ethyl acetate. The resulting mixture was distilled under a reduced pressure, and the resulting residue was subjected to silica gel column chromatography (dichloromethane:methanol=4:1) to obtain 5-(piperazin-1-yl)picolinonitrile (yield: 68%).

¹H NMR (200 MHz, CDCl₃) δ 8.31 (d, 1H, J=2.8 Hz), 7.51 (d, 1H, J=9.0 Hz), 7.08 (dd, 1H, J=2.9, 8.9 Hz), 3.37-3.22 (m, 4H), 3.07-3.01 (m, 4H), 1.76 (brs, 1H); MS (EI) m/z C₁₀H₁₂N₄ calc. 188.11, found 188 (M⁺, 23), 146 (63), 120 (100).

Step 2

The procedure of Step 2 of Example 1 was repeated except for using the compound obtained in Step 1 instead of 2-(piperazin-1-yl)benzooxazole to obtain the title compound (yield: 34%).

¹H NMR (300 MHz, CDCl₃) δ 8.31 (d, 1H, J=2.8 Hz), 7.90 (s, 1H), 7.79 (s, 1H), 7.54-7.38 (m, 2H), 7.12-7.06 (m, 1H), 6.82-6.67 (m, 2H), 5.01 (br s, 1H), 4.94-4.85 (m, 2H), 3.43-3.30 (m, 4H), 3.19-3.04 (m, 3H), 2.67-2.58 (m, 2H), 0.94 (d, 3H, J=6.8 Hz); MS (EI) m/z C₂₂H₂₃F₂N₇O calc. 439.19, found 440 (M⁺+1, 1), 357 (87), 215 (100).

### EXAMPLE 18

### Preparation of (2R,3R)-2-(2,4-difluorophenyl)-3-(4-(pyrimidin-2-yl)piperazin-1-yl)-1-(1H-1,2,4-triazol-1-yl)butan-2-ol

Step 1

The procedure of Step 1 of Example 2 was repeated except for using 2-chloropyrimidine instead of 2-chlorobenzothiazole and conducting the reaction for 30 mins to obtain 2-(piperazin-1-yl)pyrimidine (yield: 68%).

¹H NMR (300 MHz, MeOD-d₄) δ 8.53 (dd, 2H, J=1.5, 4.8 Hz), 6.80 (dt, 1H, J=1.5, 4.8 Hz), 4.01-3.97 (m, 4H), 3.10-3.06 (m, 4H); MS (EI) m/z C₈H₁₂N₄ calc. 164.11, found 164 (M⁺, 22), 122 (100), 96 (66).

Step 2

The procedure of Step 2 of Example 1 was repeated except for using the compound obtained in Step 1 instead of 2-(piperazin-1-yl)benzooxazole to obtain the title compound (yield: 41%).

¹H NMR (200 MHz, CDCl₃) δ 8.30 (d, 2H, J=4.4 Hz), 7.97 (s, 1H), 7.79 (s, 1H), 7.54-7.42 (m, 1H), 6.82-6.68 (m, 2H), 6.51-6.46 (m, 1H), 5.21 (br s, 1H), 4.99 (d, 1H, J=14.2 Hz), 4.88 (d, 1H, J=14.6 Hz), 3.83 (br s, 4H), 3.07-2.85 (m, 3H), 2.54-2.43 (m, 2H), 0.94 (d, 3H, J=7.0 Hz).

### EXAMPLE 19

### Preparation of (2R,3R)-2-(2,4-difluorophenyl)-3-(4-(quinolin-2-yl)piperazin-1-yl)-1-(1H-1,2,4-triazol-1-yl)butan-2-ol

Step 1

0.20 g (2.45 mmol) of piperazine was dissolved in 15 mL of isopropanol in a dried round flask provided with nitrogen gas, 2-chloroquinoline 0.20 g (1.2 mmol) was added thereto, and the mixture was refluxed with stirring for 24 hrs. After adding water thereto, the reaction was extracted with ethyl acetate. The formed organic layer was washed with a saturated NaCl solution, dried over anhydrous magnesium sulfate, and concentrated under a reduced pressure. The resulting residue was subjected to silica gel column chromatography (dichloromethane:methanol=4:1) to obtain 2-(piperazin-1-yl)quinoline (yield: 36%).

¹H NMR (300 MHz, CDCl₃) δ 7.87 (d, 1H, J=9.0 Hz), 7.65 (d, 1H, J=15.2 Hz), 7.55 (tt, 1H, J=1.4, 7.1 Hz), 7.18-7.26 (m, 1H), 6.95 (d, 1H, J=9.2 Hz), 3.70 (t, 4H, J=5.0 Hz), 3.00 (t, 4H, J=5.4 Hz), 2.04 (br s, 1H); MS (EI) m/z C₁₃H₁₅N₃ calc. 213.13, found 213 (M⁺, 22), 171 (63), 145 (100).

Step 2

The procedure of Step 2 of Example 1 was repeated except for using the compound obtained in Step 1 instead of 2-(piperazin-1-yl)benzooxazole to obtain the title compound (yield: 38%).

¹H NMR (200 MHz, CDCl₃) δ 7.98 (s, HA 7.88 (d, 1H, J=9.4 Hz), 7.80 (s, 1H), 7.70 (d, 1H, J=7.6 Hz), 7.43-7.62 (m, 3H), 7.18-7.26 (m, 1H), 6.96 (d, 1H, J=9.2 Hz), 6.70-6.82 (m, 2H), 5.15 (br s, 1H), 4.99 (q, 2H, J=14.6, 20.8 Hz), 3.74 (br s, 4H), 2.91-2.97 (m, 3H), 2.51-2.59 (m, 2H), 0.97 (d, 3H, J=6.8 Hz).

### EXAMPLE 20

### Preparation of (2R,3R)-3-(4-(6-(benzyloxy)pyridin-2-yl)piperazin-1-yl)-2-(2,4-difluorophenyl)-1-(1H-1,2,4-triazol-1-yl)butan-2-ol

Step 1

0.30 g (12 mmol) of sodium hydride was dispersed in anhydrous THF in a dried flask provided with nitrogen gas, benzylalcohol (12 mmol) was added thereto, and the mixture was stirred for 30 mins. After adding 2.4 g (10 mmol) of 2,6-dibromopyridine thereto, the reaction mixture was kept at room temperature for 18 hrs, and the reaction was completed by adding water thereto. The resulting solution was extracted 3 times with ethyl acetate, and the formed organic layer was washed with a saturated NaCl solution, dried over anhydrous magnesium sulfate, and concentrated under a reduced pressure. The resulting residue was subjected to silica gel column chromatography (n-hexane:ethylacetate=19:1) to obtain 2-benzyloxy-6-bromopyridine (yield: 90%).

¹H NMR (300 MHz, CDCl₃) δ 7.30-7.48 (m, 6H), 7.07 (d, 1H, J=7.5 Hz), 6.73 (d, 1H, J=8.2 Hz), 5.36 (s, 2H).

Step 2

0.50 g (1.9 mmol) of the compound obtained in Step 1 was placed in a microwave reactor dried with nitrogen gas, 0.53 g (2.9 mmol) of tert-butyl piperidine-1-carboxylate, 17 mg (1 mol %) of tris(dibenzylideneacetone)dipalladium, 18 mg (1.5 mol %) of BINAP [(±)-2,2′-bis(diphenylphosphino)-1,1′-binaphthyl], 0.25 g (2.6 eq) of sodium tert-butoxide and 4 mL of toluene was added thereto, and the reactor was sealed with a septum. The reactor was kept at 120° C. for 10 mins and cooling to room temperature, followed filtering the reaction mixture through a cellite in concurrence with washing with ethyl acetate. The resulting mixture was distilled under a reduced pressure, and the resulting residue was subjected to silica gel column chromatography (n-hexane:ethylacetate=19:1) to obtain tent-butyl 4-(6-(benzyloxy)pyridin-2-yl)piperazine-1-carboxylate (yield: 71%).

¹H NMR (200 MHz, CDCl₃) δ 7.41-7.27 (m, 5H), 6.90 (br s, 3H), 5.02 (s, 2H), 3.60-3.55 (m, 4H), 3.01-2.99 (m, 4H), 1.49 (s, 9H).

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain 1-(6-(benzyloxy)pyridin-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 35%).

¹H NMR (300 MHz, CDCl₃) δ 7.96 (s, 1H), 7.78 (s, 1H), 7.47-7.25 (m, 6H), 6.93-6.74 (m, 5H), 5.28 (br s, 1H), 5.01 (s, 2H), 4.93 (d, J=14.4 Hz, 1H), 4.83 (d, J=14.4 Hz, 1H), 3.08-2.92 (m, 7H), 2.61-2.54 (m, 2H), 0.94 (d, 3H, J=7.0 Hz); MS (EI) m/z C₂₈H₃₀F₂N₆O₂ calc. 520.24, found 520 (M⁺, 1), 295 (100), 140 (15).

### EXAMPLE 21

### Preparation of (2R,3R)-3-(4-(6-(cyclopropylmethoxy)pyridin-2-yl)piperazin-1-yl)-2-(2,4-difluorophenyl)-1-(1H-1,2,4-triazol-1-yl)butan-2-ol

Step 1

The procedure of Step 1 of Example 20 was repeated except for using cyclopropylmethanol instead of benzylalcohol to obtain 2-bromo-6-(cyclopropylmethoxy)pyridine (yield: 78%).

¹H NMR (200 MHz, CDCl₃) δ 7.42 (t, 1H, J=7.9 Hz), 7.05 (d, 1H, J=7.4 Hz), 6.72 (d, 1H, J=8.2 Hz), 4.14 (d, 2H, J=7.0 Hz), 1.18-1.36 (m, 1H), 0.66-0.61 (m, 1H), 0.38-0.33 (m, 1H).

Step 2

The procedure of Step 2 of Example 20 was repeated except for using the compound obtained in Step 1 instead of 2-benzyloxy-6-bromopyridine to obtain tert-butyl 4-(6-(cyclopropylmethoxy)pyridin-2-yl)piperazine-1-carboxylate compound (yield: 84%).

¹H NMR (200 MHz, CDCl₃) δ 7.40 (t, 1H, J=7.9 Hz), 6.16-6.10 (m, 2H), 4.05 (d, 2H, J=7.0 Hz), 3.55-3.45 (m, 8H), 1.48 (s, 9H), 1.26 (t, 1H, J=7.1 Hz), 0.65-0.55 (m, 2H), 0.37-0.29 (m, 2H).

Step 3

The procedure of Step 3 of Example 5 was repeated except for using the compound obtained in Step 2 instead of tert-butyl-4-(5-chlorobenzooxazol-2-yl)piperazine-1-carboxylate to quantitatively obtain 1-(6-(cyclopropylmethoxy)pyridine-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 46%).

¹H NMR (300 MHz, CDCl₃) δ 7.99 (s, 1H), 7.80 (s, 1H), 7.51-7.49 (m, 1H), 7.41 (t, 1H, J=7.9 Hz), 6.81-6.77 (m, 2H), 6.13 (t, 2H, J=7.4 Hz), 5.25 (br s, 1H), 4.99-4.82 (m, 2H), 4.07 (d, 2H, J=7.1 Hz), 3.51 (br s, 4H), 3.02-2.80 (m, 3H), 2.52 (br s, 2H), 1.30-1.27 (m, 1H), 0.99-0.98 (m, 3H), 0.62-0.59 (m, 2H), 0.37-0.33 (m, 2H); MS (EI) m/z C₂₅H₃₀F₂N₆O₂ calc. 484.24, found 484 (M⁺, 1), 260 (100), 224 (2), 206 (14).

### EXAMPLE 22

### Preparation of (2R,3R)-3-(4-(6-(cyclopentyloxy)pyridin-2-yl)piperazin-1-yl)-2-(2,4-difluorophenyl)-1-(1H-1,2,4-triazol-1-yl)butan-2-ol

Step 1

The procedure of Step 1 of Example 20 was repeated except for using cyclopentanol instead of benzylalcohol to obtain 2-bromo-6-(cyclopentyloxy)pyridine (yield: 82%).

¹H NMR (200 MHz, CDCl₃) δ 7.38 (t, 1H, J=7.8 Hz), 7.70 (d, 1H, J=7.4 Hz), 6.61 (d, 1H, J=8.2 Hz), 5.33-5.40 (m, 1H), 1.26-2.04 (m, 8H).

Step 2

The procedure of Step 2 of Example 20 was repeated except for using the compound obtained in Step 1 instead of 2-bromo-6-benzyloxypyridine to obtain tert-butyl 4-(6-(cyclopentyloxy)pyridin-2-yl)piperazine-1-carboxylate (yield: 68%).

¹H NMR (200 MHz, CDCl₃) δ 7.42 (t, 1H, J=8.1 Hz), 6.25 (d, 1H, J=8.6 Hz), 6.11 (d, 1H, J=7.6 Hz), 5.30-5.21 (m, 1H), 3.56-3.28 (m, 8H), 1.97-1.71 (m, 8H), 1.48 (s, 9H).

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain 1-(6-(cyclopentyloxy)pyridin-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 27%).

¹H NMR (300 MHz, MeOD-d₃) δ 8.28 (s, 1H), 7.73 (s, 1H), 7.43-7.36 (m, 2H), 6.90-6.81 (m, 2H), 6.22 (d, 1H, J=7.8 Hz), 5.99 (d, 1H, J=7.8 Hz), 5.29-5.27 (m, 1H), 5.05-4.92 (m, 2H), 3.54 (br s, 4H), 3.27-3.24 (m, 1H), 3.03-2.99 (m, 2H), 2.63-2.59 (m, 2H), 1.95-1.92 (m, 2H), 1.77-1.61 (m, 6H), 0.91 (d, 3H, J=6.6 Hz); MS (EI) m/z C₂₆H₃₂F₂N₆O₂ calc. 498.26, found 498 (M⁺, 1), 274 (100), 224 (3), 163 (14).

### EXAMPLE 23

### Preparation of (2R,3R)-3-(4-(6-(butyloxy)pyridin-2-yl)piperazin-1-yl)-2-(2,4-difluorophenyl)-1-(1H-1,2,4-triazol-1-yl)butan-2-ol

Step 1

The procedure of Step 1 of Example 20 was repeated except for using n-butaneol instead of benzylalcohol to obtain 2-bromo-6-(butyloxy)pyridine (yield: 91%).

¹H NMR (200 MHz, CDCl₃) δ 7.40 (t, 1H, J=7.8 Hz), 7.03 (d, 1H, J=7.4 Hz), 6.66 (d, 1H, J=7.8 Hz), 4.28 (t, 2H, J=6.6 Hz), 1.81-1.67 (m, 2H), 1.56-1.37 (m, 2H), 0.97 (t, 3H, J=7.2 Hz).

Step 2

The procedure of Step 2 of Example 20 was repeated except for using the compound obtained in Step 1 instead of 2-bromo-6-benzyloxypyridine to obtain tert-butyl 4-(6-butoxypyridin-2-yl)piperazine-1-carboxylate (yield: 78%).

¹H NMR (200 MHz, CDCl₃) δ 7.42 (t, 1H, J=7.9 Hz), 6.21-6.09 (m, 2H), 4.23 (t, 2H, J=6.5 Hz), 3.51-3.53 (m, 8H), 1.56-1.81 (2H, m), 1.48 (s, 9H), 0.97 (t, 3H, J=7.3 Hz).

Step 3

The procedure of Step 3 of Example 5 was repeated except for using the compound obtained in Step 2 instead of tent-butyl-4-(5-chlorobenzooxazol-2-yl)piperazine-1-carboxylate to quantitatively obtain 1-(6-(butyloxy)pyridin-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 39%).

¹H NMR (300 MHz, CDCl₃) δ 7.97 (s, 1H), 7.79 (s, 1H), 7.47 (dd, 1H, J=6.6, 9.0 Hz), 7.39 (t, 1H, J=7.9 Hz), 6.82-6.71 (m, 2H), 6.14-6.05 (m, 2H), 5.24 (s, 1H), 4.95 (d, 1H, J=14.7 Hz), 4.85 (d, 1H, J=15.3 Hz), 4.22 (t, 2H, J=6.6 Hz), 3.50 (br s, 4H), 3.01-2.89 (m, 3H), 2.54-2.50 (m, 2H), 1.76-1.69 (m, 2H), 1.50-1.42 (m, 2H), 0.99-0.94 (m, 6H); MS (EI) m/z C₂₅H₃₂F₂N₆O₂ calc. 486.26, found 486 (M⁺, 1), 262 (100), 163 (12), 137 (6).

### EXAMPLE 24

### Preparation of (2R,3R)-2-(2,4-difluorophenyl)-3-(4-(6-(isopropyloxy)pyridin-2-yl)piperazin-1-yl)-1-(1H-1,2,4-triazol-1-yl)butan-2-ol

Step 1

The procedure of Step 1 of Example 20 was repeated except for using isopropanol instead of benzylalcohol to obtain 2-bromo-6-(isopropyloxy)pyridine (yield: 79%).

¹H NMR (200 MHz, CDCl₃) δ 7.38 (t, 1H, J=7.8 Hz), 7.00 (d, 1H, J=7.8 Hz), 6.61 (d, 1H, J=8.2 Hz), 5.35-5.22 (m, 1H), 1.33 (d, 6H, J=6.0 Hz).

Step 2

The procedure of Step 2 of Example 20 was repeated except for using the compound obtained in Step 1 instead of 2-bromo-6-benzyloxypyridine to obtain tert-butyl 4-(6-isopropyloxypyridin-2-yl)piperazine-1-carboxylate (yield: 74%).

¹H NMR (200 MHz, CDCl₃) δ 7.46 (t, 1H, J=7.9 Hz), 6.33 (d, 1H, J=8.2 Hz), 6.16 (d, 1H, J=7.2 Hz), 5.25-5.19 (m, 1H), 3.60-3.50 (m, 8H), 1.51 (s, 9H), 1.36 (d, 6H, J=6.2 Hz).

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain 1-(6-(isopropyloxy)pyridin-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 27%).

¹H NMR (300 MHz, CDCl₃) δ 7.97 (s, 1H), 7.79 (s, 1H), 7.53-7.47 (m, 1H), 7.40-7.35 (m, 1H), 6.81-6.71 (m, 2H), 6.11 (d, 1H, J=8.1 Hz), 6.03 (d, 1H, J=7.8 Hz), 5.24-5.17 (m, 2H), 5.02-4.88 (m, 2H), 3.49 (br s, 4H), 3.01-2.90 (m, 3H), 2.54-2.50 (m, 2H), 1.33 (d, 6H, J=6.3 Hz), 0.97 (d, 3H, J=6.9 Hz); MS (EI) m/z C₂₄H₃₀F₂N₆O₂ calc. 472.24, found 472 (M⁺, 1), 248 (100), 206 (8), 163 (25).

### EXAMPLE 25

### Preparation of (2R,3R)-2-(2,4-difluorophenyl)-3-(4-(6-(thiophen-2-ylmethoxy)pyridin-2-yl)piperazin-1-yl)-1-(1H-1,2,4-triazol-1-yl)butan-2-ol

Step 1

The procedure of Step 1 of Example 20 was repeated except for using thiophen-2-ylmethanol instead of benzylalcohol to obtain 2-((thiophen-2-yl)methoxy)-6-bromopyridine (yield: 88%).

¹H NMR (200 MHz, CDCl₃) δ 7.43 (t, 1H, J=7.7 Hz), 7.33 (dd, 1H, J=1.0, 5.2 Hz), 7.19-7.17 (m, 1H), 7.09 (d, 1H, J=7.6 Hz), 7.00 (dd, 1H, J=3.4, 5.0 Hz), 6.72 (d, 1H, J=8.0 Hz), 5.53 (s, 2H).

Step 2

The procedure of Step 2 of Example 20 was repeated except for using the compound obtained in Step 1 instead of 2-(benzyloxy)-6-bromopyridine and piperazine instead of tent-butyl piperazine-1-carboxylate to obtain 1-(6-((thiophen-2-yl)methoxy)pyridin-2-yl)piperazine (yield: 54%).

¹H NMR (200 MHz, CDCl₃) δ 7.41 (t, 1H, J=8.1 Hz), 7.27 (dd, 1H, J=0.8, 5.6 Hz), 7.13-7.10 (m, 1H), 6.97 (dd, 1H, J=3.5, 5.1 Hz), 6.17 (d, 1H, J=8.0 Hz), 6.12 (d, 1H, J=8.0 Hz), 5.50 (s, 2H), 3.58-3.53 (m, 4H), 3.04-2.99 (m, 4H), 2.43 (br s, 1H).

Step 3

The procedure of Step 2 of Example 1 was repeated except for using the compound obtained in Step 2 instead of 2-(piperazin-1-yl)benzooxazole and propionitrilenitrile instead of acetonitrile to obtain the title compound (yield: 47%).

¹H NMR (300 MHz, CDCl₃) δ 7.97 (s, 1H), 7.79 (s, 1H), 7.46 (dd, 1H, J=2.4, 9.3 Hz), 7.39 (t, 1H, J=8.1 Hz), 7.26-7.28 (m, 1H), 7.10-7.11 (d, J=3.3 Hz, 1H), 6.69-6.99 (m, 2H), 6.10 (dd, J=7.8, 14.4 Hz, 2H), 5.49 (s, 2H), 5.21 (s, 1H), 4.94 (d, J=14.5 Hz, 1H), 4.85 (d, J=14.6 Hz, 1H), 3.55 (br s, 4H), 2.91-3.05 (m, 2H), 2.50-2.57 (m, 2H), 0.98 (d, J=6.8 Hz, 3H); MS (EI) m/z C₂₆H₂₈F₂N₆O₂S calc. 526.20, found 526 (M⁺, 3), 149 (60), 96 (100).

### EXAMPLE 26

### Preparation of (2R,3R)-2-(2,4-difluorophenyl)-3-(4-(6-(2-morpholinoethoxy)pyridin-2-yl)piperazin-1-yl)-1-(1H-1,2,4-triazol-1-yl)butan-2-ol

Step 1

The procedure of Step 1 of Example 20 was repeated except for using 2-morpholinoethanol instead of benzylalcohol to obtain 2-bromo-6-(2-morpholinoethoxy)pyridine (yield: 97%).

¹H NMR (300 MHz, CDCl₃) δ 7.41 (t, 1H, J=7.9 Hz), 7.05 (d, 1H, J=7.4 Hz), 6.70 (d, 1H, J=8.0 Hz), 4.44 (t, 2H, J=5.7 Hz), 3.70-3.76 (m, 4H), 2.77 (t, 2H, J=5.8 Hz), 2.54-2.58 (m, 4H).

Step 2

The procedure of Step 2 of Example 20 was repeated except for using the compound obtained in Step 1 instead of 2-bromo-6-benzyloxypyridine to obtain tert-butyl 4-(6-(2-morpholinoethoxy)pyridin-2-yl)piperazine-1-carboxylate (yield: 69%).

¹H NMR (200 MHz, CDCl₃) δ 7.41 (t, 1H, J=7.9 Hz), 6.13 (t, 2H, J=7.9 Hz), 4.39 (t, 2H, J=5.9 Hz), 3.76-3.71 (m, 4H), 3.51-3.50 (m, 8H), 2.77 (t, 2H, J=6.1 Hz), 2.59-2.54 (m, 4H), 1.49 (s, 9H).

Step 3

The procedure of Step 3 of Example 5 was repeated except for using the compound obtained in Step 2 instead of tert-butyl 4-(5-chlorobenzooxazol-2-yl)piperazine-1-carboxylate to quantitatively obtain 1-(6-(2-morpholinoethoxy)pyridin-2-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 47%).

¹H NMR (300 MHz, CDCl₃) δ 7.97 (s, 1H), 7.78 (s, 1H), 7.76 (d, 1H, J=2.7 Hz), 7.48-7.45 (m, 1H), 7.29-7.25 (m, 1H), 6.80-6.68 (m, 3H), 5.15 (br s, 1H), 4.94 (d, 1H, J=14.4 Hz), 4.84 (d, 1H, J=14.4 Hz), 4.39 (t, 2H, 5.7 Hz), 3.73 (t, 4H, J=4.8 Hz), 3.09-2.99 (m, 7H), 2.78 (t, 2H, J=5.7 Hz), 2.64-2.55 (m, 6H), 0.98 (d, 3H, J=6.9 Hz); MS (EI) m/z C₂₇H₃₅F₂N₇O₃ calc. 543.28, found 543 (M⁺, 1), 319 (100), 114 (29).

### EXAMPLE 27

### Preparation of (2R,3R)-2-(2,4-difluorophenyl)-3-(4-(6-(methyl(3-morpholinopropyl)amino)pyridin-2-yl)piperazin-1-yl)-1-(1H-1,2,4-triazol-1-yl)butan-2-ol

Step 1

95 mg (0.4 mmol) of 2,6-dibromopyridine and 86 mg (0.6 mmol) of 3-morpholinopropylamine were placed in a dried 5 ml microwave reactor provided with nitrogen gas, followed by keeping the reactor at 150° C. for 20 mins. After cooling to room temperature, the reaction mixture was filtered through a cellite in concurrence with washing with ethyl acetate. The resulting solution was distilled under a reduced pressure, and the resulting residue was subjected to silica gel column chromatography (n-hexane:ethyl acetate=19:1) to obtain 2-bromo-6-(3-morpholinopropylamino)pyridine (yield: 90%).

¹H NMR (200 MHz, CDCl₃) δ 7.20 (d, 1H, J=7.6 Hz), 6.69 (d, 1H, J=7.6 Hz), 6.27 (d, 1H, J=8.4 Hz), 5.39 (br s, 1H), 3.73 (t, 4H, J=4.6 Hz), 3.34 (q, 2H, J=6.2 Hz), 2.50-2.43 (m, 6H), 1.77 (quint, 2H, J=6.6 Hz).

Step 2

30 tag (1.2 mmol) of sodium hydride, DMF, and the compound obtained in Step 1 were placed in a flask dried with nitrogen gas, and the mixture was stirred for 30 mins. After adding 0.17 g (1.2 mmol) of iodomethane thereto, the reaction mixture was kept at room temperature for 4 hrs, followed by completing the reaction by adding water thereto. The resulting mixture was extracted 3 times with ethyl acetate, and the formed organic layer was washed with a saturated NaCl solution, dried over anhydrous magnesium sulfate, and concentrated under a reduced pressure. The resulting residue was subjected to silica gel column chromatography (n-hexane:ethylacetate=19:1) to obtain 6-bromo-N-methyl-N-(3-morpholinopropyl)pyridine-2-amine (yield: 76%).

¹H NMR (200 MHz, CDCl₃) δ 7.22 (t, 1H, J=8.4 Hz), 6.64 (d, 1H, J=7.2 Hz), 6.39 (d, 1H, J=8.2 Hz), 3.76-3.70 (m, 4H), 3.55 (t, 2H, J=7.2 Hz), 3.02 (s, 3H), 2.46-2.32 (m, 6H), 1.76 (quint, 2H, J=7.2 Hz).

Step 3

The procedure of Step 2 of Example 18 was repeated except for using the compound obtained in Step 2 instead of 2-bromo-6-benzyloxypyridine and 2-dicyclohexylphosphino-2′-(N,N-dimethylamino)biphenyl instead of tert-butyl 4-(6-(N-methyl-N-(3-morpholinopropyl)amino)pyridin-2-yl)piperazine-1-carboxylate (yield: 74%).

¹H NMR (200 MHz, CDCl₃) δ 7.30 (t, 1H, J=8.2 Hz), 5.90 (d, 2H, J=8.0 Hz), 3.74-3.69 (m, 4H), 3.56-3.45 (m 10H), 2.99 (s, 3H), 2.45-2.32 (m, 6H), 1.85-1.74 (m, 2H), 1.48 (s, 9H).

Step 4

The procedure of Step 3 of Example 5 was repeated except for using the compound obtained in Step 3 instead of tert-butyl 4-(5-chlorobenzooxazol-2-yl)piperazine-1-carboxylate to quantitatively obtain N-methyl-N-(3-morpholinopropyl)-6-(piperazin-1-yl)pyridine-2-amine.

Step 5

The procedure of Step 2 of Example 1 was repeated except for using the compound obtained in Step 4 instead of 2-(piperazin-1-yl)benzooxazole and propionitrilenitrile instead of acetonitrile to obtain the title compound (yield: 35%).

¹H NMR (300 MHz, CDCl₃) δ 7.98 (s, 1H), 7.79 (s, 1H), 7.50-7.47 (m, 1H), 7.31-7.26 (m, 1H), 6.82-6.71 (m, 2H), 5.88 (d, J=8.1 Hz, 2H), 5.05 (br s, 1H), 4.94 (d, J=14.5 Hz, 1H), 4.84 (d, J=14.6 Hz, 1H), 3.71 (t, J=4.6, 4H), 3.55-3.50 (m, 6H), 3.02-2.96 (m, 3H), 2.90-2.83 (m, 2H), 2.53-2.33 (m, 9H), 1.79-1.74 (m, 2H), 0.97 (d, J=6.2 Hz, 3H); MS (EI) m/z C₂₉H₄₀F₂N₈O₂ calc. 570.32, found 570 (M⁺, 2), 346 (75), 128 (37), 100 (100).

### EXAMPLE 28

### Preparation of (2R,3R)-2-(2,4-difluorophenyl)-3-(4-(6-(methyl(2-(thiophen-2-yl)ethyl)amino)pyridin-2-yl)piperazin-1-yl)-1-(1H-1,2,4-triazol-1-yl)butan-2-ol

Step 1

The procedure of Step 1 of Example 27 was repeated except for using 2-(thiophen-2-yl)ethylamine instead of 3-morpholinopropylamine to obtain 2-bromo-6-(2-(thiophen-2-yl)ethylamino)pyridine (yield: 84%).

¹H NMR (200 MHz, CDCl₃) δ 7.28-7.15 (m, 2H), 6.97-6.92 (m, 1H), 6.86-6.84 (m, 1H), 6.73 (d, 1H, J=8.2 Hz), 6.28 (d, 1H, J=8.0 Hz), 4.75 (br s, 1H), 3.56 (q, 2H, J=6.5 Hz), 3.12 (t, 2H, J=6.7 Hz).

Step 2

The procedure of Step 2 of Example 27 was repeated except for using the compound obtained in Step 1 instead of 2-bromo-6-(3-morpholinopropylamino)pyridine to obtain 6-bromo-N-methyl-N-(2-(thiophen-2-yl)ethyl)pyridine-2-amine (yield: 91%).

¹H NMR (200 MHz, CDCl₃) δ 7.28-7.12 (m, 2H), 6.95-6.91 (m, 1H), 6.84-6.82 (m, 1H), 6.68 (d, 1H, J=7.4 Hz), 6.34 (d, 1H, J=8.4 Hz), 3.77 (t, 2H, J=7.3 Hz), 3.11 (t, 2H, J=7.3 Hz), 2.97 (s, 3H).

Step 3

The procedure of Step 3 of Example 27 was repeated except for using the compound obtained in Step 2 instead of 6-bromo-N-methyl-N-(3-morpholinopropyl)pyridine-2-amine and piperazine instead of tert-butyl 4-piperazine-1-carboxylate to obtain N-methyl-N-(3-thiophen-2-yl)ethyl-6-(piperazin-1-yl)pyridine-2-amine (yield: 64%).

¹H NMR (200 MHz, CDCl₃) δ 7.33 (t, 1H, J=7.8 Hz), 7.13 (dd, 1H, J=1.4, 5.2 Hz), 6.93 (m, 1H), 6.80-6.82 (m, 1H), 5.91 (t, 2H, J=8.6 Hz), 3.75-3.80 (m, 2H), 3.58-3.63 (m, 4H), 3.04-3.13 (m, 6H), 2.17 (br s, 3H).

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 62%).

¹H NMR (300 MHz, CDCl₃) δ 7.99 (s, 1H), 7.79 (s, 1H), 7.49-7.51 (m, 1H), 7.32 (t, J=8.1 Hz, 1H), 7.14 (dd, 1H, J=0.75, 4.9 Hz), 6.92-6.95 (m, 1H), 6.74-6.81 (m, 3H), 5.90 (q, J=8.2 Hz, 2H), 5.30 (br s, 1H), 4.94 (d, J=14.6 Hz, 1H), 4.84 (d, J=14.6 Hz, 1H), 3.73-3.78 (m, 2H), 3.52 (br s, 4H), 3.07-3.12 (m, 2H), 2.96-2.97 (m, 4H), 2.84-2.88 (m, 2H), 2.48-2.52 (m, 2H), 0.98 (d, J=6.9 Hz, 3H); MS (EI) m/z C₂₈H₃₃F₂N₇OS calc. 553.24, found 553 (M⁺, 2), 329 (100), 116 (9).

### EXAMPLE 29

### Preparation of (2R,3R)-3-(4-(6-(benzyloxy)pyridin-3-yl)piperazin-1-yl)-2-(2,4-difluorophenyl)-1-(1H-1,2,4-triazol-1-yl)butan-2-ol

Step 1

The procedure of Step 1 of Example 20 was repeated except for using 2,5-dibromopyridine instead of 2,6-dibromopyridine to obtain 2-benzyloxy-5-bromopyridine (yield: 87%).

¹H NMR (200 MHz, CDCl₃) δ 8.21 (s, 1H), 7.66 (d, 1H, J=8.8 Hz), 7.47-7.30 (m, 5H), 6.73 (d, 1H, J=8.8 Hz), 5.35 (s, 2H).

Step 2

The procedure of Step 2 of Example 5 was repeated except for using the compound obtained in Step 1 instead of 2-benzyloxy-6-bromopyridine and conducting the reaction at 180° C. instead of 120° C. to obtain tert-butyl 4-(6-(benzyloxy)pyridin-3-yl)piperazine-1-carboxylate (yield: 57%).

¹H NMR (200 MHz, CDCl₃) δ 7.83 (d, 1H, J=2.6 Hz), 7.48-7.30 (m, 6H), 6.76 (d, 1H, J=9.0 Hz), 5.33 (s, 2H), 3.62-3.57 (m, 4H), 3.05-3.00 (m, 4H), 1.48 (s, 9H).

Step 3

The procedure of Step 3 of Example 20 was repeated except for using the compound obtained in Step 2 instead of tent-butyl 4-(5-chlorobenzooxazol-2-yl)piperazine-1-carboxylate to quantitatively obtain 1-(6-(benzyloxy)pyridin-3-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 33%).

¹H NMR (300 MHz, CDCl₃) δ 7.95 (s, 1H), 7.80-7.78 (m, 2H), 7.46-7.28 (m, 7H), 6.77-6.73 (m, 3H), 5.32 (s, 2H), 5.17 (br s, 1H), 4.95 (d, 1H, J=14.7 Hz), 4.85 (d, 1H, J=14.4 Hz), 3.10-3.00 (m, 7H), 2.64-2.57 (m, 2H), 0.98 (d, 3H, J=6.9 Hz); MS (EI) m/z C₂₈H₃₀F₂N₆O₂ calc. 520, found 520 (M⁺, 1), 385 (3), 296 (100), 91 (20).

### EXAMPLE 30

### Preparation of (2R,3R)-3-(4-(6-(cyclopropylmethoxy)pyridin-3-yl)piperazin-1-yl)-2-(2,4-difluorophenyl)-1-(1H-1,2,4-triazol-1-yl)butan-2-ol

Step 1

The procedure of Step 1 of Example 20 was repeated except for using 2,5-dibromopyridine instead of 2,6-dibromopyridine and cyclopropylmethanol instead of benzylalcohol to obtain 5-bromo-2-cyclopropylmethoxypyridine (yield: 78%).

¹H NMR (200 MHz, CDCl₃) δ 8.15 (s, 1H), 7.67-7.60 (m, 1H), 6.70 (d, 1H, J=8.8 Hz), 4.09 (d, 2H, J=7.2 Hz), 1.35-1.17 (m, 1H), 0.67-0.57 (m, 2H), 0.39-0.31 (m, 2H).

Step 2

The procedure of Step 2 of Example 20 was repeated except for using the compound obtained in Step 1 instead of 2-benzyloxy-6-bromopyridine and conducting the reaction at 180° C. instead of 120° C. to obtain tert-butyl 4-(6-(cyclopropylmethoxy)pyridin-3-yl)piperazine-1-carboxylate (yield: 59%).

¹H NMR (200 MHz, CDCl₃) δ 7.77 (d, 1H, J=2.8 Hz), 7.34-7.30 (m, 1H), 6.73 (d, 1H, J=9.0 Hz), 4.07 (d, 2H, J=6.8 Hz), 3.58 (t, 4H, J=5.2 Hz), 3.00 (t, 4H, J=−5.0 Hz), 1.48 (s, 9H), 1.30-1.20 (m, 1H), 0.65-0.56 (m, 2H), 0.37-0.29 (m, 2H).

Step 3

The procedure of Step 3 of Example 5 was repeated except for using the compound obtained in Step 2 instead of tert-butyl-4-(5-chlorobenzooxazol-2-yl)piperazine-1-carboxylate to quantitatively obtain 1-(6-(cyclopropylmethoxy)pyridin-3-yl)piperazine.

Step 4

The procedure of Step 2 of Example 1 was repeated except for using the compound obtained in Step 3 instead of 5-chloro-2-thiobenzoo2-(piperazin-1-yl)benzooxazole and propionitrilenitrile instead of acetonitrile to obtain the title compound (yield: 19%).

¹H NMR (300 MHz, CDCl₃) δ 7.94 (s, 1H), 7.78 (s, 1H), 7.75 (d, 1H, J=2.8 Hz), 7.48-7.43 (m, 1H), 7.29-7.25 (m, 1H), 6.81-6.69 (m, 3H), 5.13 (s, 1H), 4.94 (d, 1H, J=14.6 Hz), 4.84 (d, 1H, J=15.2 Hz), 4.07 (d, 2H, J=7.1 Hz), 3.08-2.99 (m, 7H), 2.63-2.58 (m, 2H), 1.26-1.25 (m, 1H), 0.98 (d, 3H, J=6.9 Hz), 0.63-0.57 (m, 2H), 0.36-0.32 (m, 2H); MS (EI) m/z C₂₅H₃₀F₂N₆O₂ calc. 484, found 484 (M⁺, 1), 260 (100), 140 (5).

### EXAMPLE 31

### Preparation of (2R,3R)-3-(4-(6-(cyclopentyloxy)pyridin-3-yl)piperazin-1-yl)-2-(2,4-difluorophenyl)-1-(1H-1,2,4-triazol-1-yl)butan-2-ol

Step 1

The procedure of Step 1 of Example 20 was repeated except for using 2,5-dibromopyridine instead of 2,6-dibromopyridine and cyclopentanol instead of benzylalcohol to obtain 5-bromo-2-cyclopentyloxypyridine (yield: 78%).

¹H NMR (200 MHz, CDCl₃) δ 8.18 (d, 1H, J=2.4 Hz), 7.60 (dd, 1H, J=2.6, 8.8 Hz), 6.58 (d, 1H, J=9.0 Hz), 5.36-5.28 (m, 1H), 2.03-1.57 (m, 8H).

Step 2

The procedure of Step 2 of Example 20 was repeated except for using the compound obtained in Step 1 instead of 2-benzyloxy-6-bromopyridine and conducting the reaction at 180° C. instead of 120° C. to obtain text-butyl 4-(6-(cyclopentyloxy)pyridin-3-yl)piperazine-1-carboxylate (yield: 58%).

¹H NMR (200 MHz, CDCl₃) δ 7.88 (d, 1H, J=3.0 Hz), 7.40-7.35 (m, 1H), 6.72 (d, 1H, J=9.4 Hz), 5.35-5.41 (m, 1H), 3.66 (t, 4H, J=5.0 Hz), 3.08 (t, 4H, J=5.0 Hz), 2.04-1.68 (m, 8H), 1.56 (s, 9H).

Step 3

The procedure of Step 3 of Example 5 was repeated except for using the compound obtained in Step 2 instead of tent-butyl-4-(5-chlorobenzooxazol-2-yl)piperazine-1-carboxylate to quantitatively obtain 1-(6-(cyclopentyloxy)pyridin-3-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 21%).

¹H NMR (300 MHz, CDCl₃) δ 7.95 (s, 1H), 7.79-7.78 (m, 2H), 7.48-7.45 (m, 1H), 7.27-7.23 (m, 1H), 6.80-6.70 (m, 2H), 6.62 (d, 1H, J=9.0 Hz), 5.31-5.27 (m, 1H), 4.93 (d, 1H, J=14.5 Hz), 4.84 (d, 1H, J=14.7 Hz), 3.08-2.98 (m, 7H), 2.63-2.58 (m, 2H), 1.95-1.94 (m, 2H), 1.81-1.75 (m, 4H), 1.64-1.63 (m, 2H), 0.98 (d, 3H, J=6.9 Hz); MS (EI) m/z C₂₆H₃₂F₂N₆O₂ calc. 498, found 498 (M⁺, 1), 274 (100), 149 (5).

### EXAMPLE 32

### Preparation of (2R,3R)-3-(4-(6-(butyloxy)pyridin-3-yl)piperazin-1-yl)-2-(2,4-difluorophenyl)-1-(1H-1,2,4-triazol-1-yl)butan-2-ol

Step 1

The procedure of Step 1 of Example 20 was repeated except for using 2,5-dibromopyridine instead of 2,6-dibromopyridine and butaneol instead of benzylalcohol to obtain 5-bromo-2-butyloxypyridine (yield: 78%).

¹H NMR (200 MHz, CDCl₃) δ 8.18 (d, 1H, J=2.6 Hz), 7.62 (dd, 1H, J=2.4, 8.4 Hz), 6.60 (d, 1H, J=9.0 Hz), 4.26 (t, 2H, J=6.6 Hz), 1.81-1.56 (m, 2H), 1.52-1.37 (m, 2H), 0.97 (t, 3H, J=7.2 Hz).

Step 2

The procedure of Step 2 of Example 20 was repeated except for using the compound obtained in Step 1 instead of 2-benzyloxy-6-bromopyridine and conducting the reaction at 180° C. instead of 120° C. to obtain tert-butyl 4-(6-(butyloxy)pyridin-3-yl)piperazine-1-carboxylate (yield: 57%).

¹H NMR (200 MHz, CDCl₃) δ 7.81 (d, 1H, J=2.2 Hz), 7.36-7.33 (m, 1H), 6.66 (d, 1H, J=9.0 Hz), 4.24 (t, 2H, J=6.8 Hz), 3.60 (t, 41-1, J=5.0 Hz), 3.02 (t, 4H, J=4.6 Hz), 1.81-1.67 (m, 2H), 1.52-1.36 (m, 1H), 0.96 (t, 3H, J=7.2 Hz).

Step 3

The procedure of Step 3 of Example 5 was repeated except for using the compound obtained in Step 2 instead of tert-butyl-4-(5-chlorobenzooxazol-2-yl)piperazine-1-carboxylate to quantitatively obtain 1-(6-(butyloxy)pyridin-3-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 45%).

¹H NMR (300 MHz, CDCl₃) δ 8.01 (s, 1H), 7.78-7.76 (m, 2H), 7.48-7.44 (m, 1H), 7.34-7.30 (m, 1H), 6.82-6.67 (m, 3H), 4.95-4.83 (m, 2H), 4.21 (t, 2H, J=6.7 Hz), 3.39-2.98 (m, 7H), 2.62 (br s, 2H), 1.79-1.70 (m, 2H), 1.54-1.44 (m, 2H), 1.01-0.94 (m, 6H); MS (EI) m/z C₂₅H₃₂F₂N₆O₂ calc. 486, found 486 (M⁺, 1), 262 (100), 205 (3), 140 (4).

### EXAMPLE 33

### Preparation of (2R,3R)-2-(2,4-difluorophenyl)-3-(4-(6-(isopropyloxy)pyridin-3-yl)piperazin-1-yl)-1-(1H-1,2,4-triazol-1-yl)butan-2-ol

Step 1

The procedure of Step 1 of Example 20 was repeated except for using 2,5-dibromopyridine instead of 2,6-dibromopyridine and isopropanol instead of benzylalcohol to obtain 5-bromo-2-isopropyloxypyridine (yield: 76%).

¹H NMR (200 MHz, CDCl₃) δ 8.17 (d, 1H, J=2.6 Hz), 7.61 (dd, 1H, J=2.7, 8.7 Hz), 6.59 (d, 1H, J=8.6 Hz), 5.29-5.17 (m, 1H, J=6.1 Hz), 1.34 (d, 6H, J=6.8 Hz).

Step 2

The procedure of Step 2 of Example 20 was repeated except for using the compound obtained in Step 1 instead of 2-benzyloxy-6-bromopyridine and conducting the reaction at 180° C. instead of 120° C. to obtain tert-butyl 4-(6-(isopropyloxy)pyridin-3-yl)piperazine-1-carboxylate (yield: 58%).

¹H NMR (200 MHz, CDCl₃) δ 7.80 (d, 1H, J=2.8 Hz), 7.33-7.28 (m, 1H), 6.64 (d, 1H, J=9.0 Hz), 5.27-5.14 (m, 1H), 3.59 (t, 4H, J=4.8 Hz), 3.01 (t, 4H, J=5.2 Hz), 1.49 (s, 9H), 1.33 (d, 61-1, J=6.0 Hz).

Step 3

The procedure of Step 3 of Example 5 was repeated except for using the compound obtained in Step 2 instead of tert-butyl-4-(5-chlorobenzooxazol-2-yl)piperazine-1-carboxylate to quantitatively obtain 1-(6-(isopropyloxy)pyridin-3-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 32%).

¹H NMR (300 MHz, CDCl₃) δ 7.95 (s, 1H), 7.78-7.77 (m, 2H), 7.48-7.43 (m, 1H), 7.27-7.23 (m, 1H), 6.80-6.70 (m, 2H), 6.62 (d, 1H, J=9.0 Hz), 5.21-5.13 (m, 2H), 4.94 (d, 1H, J=14.4 Hz), 4.84 (d, 1H, J=14.4 Hz), 3.08-2.98 (m 7H), 2.63-2.57 (m, 2H), 1.32 (d, 6H, J=6.3 Hz), 0.98 (d, 3H, J=6.9 Hz); MS (EI) m/z C₂₄H₃₀F₂N₆O₂ calc. 472, found 472 (M⁺, 1), 248 (100), 140 (23).

### EXAMPLE 34

### Preparation of (2R,3R)-2-(2,4-difluorophenyl)-3-(4-(6-(2-morpholinoethoxy)pyridin-3-yl)piperazin-1-yl)-1-(1H-1,2,4-triazol-1-yl)butan-2-ol

Step 1

The procedure of Step 1 of Example 20 was repeated except for using 2,5-dibromopyridine instead of 2,6-dibromopyridine, 2-morpholinoethanol instead of benzylalcohol, and conducting the reaction with refluxing and stirring instead of at room temperature to obtain 5-bromo-2-(2-morpholinoethoxy)pyridine (yield: 97%).

¹H NMR (200 MHz, CDCl₃) δ 8.17 (d, 1H, J=2.6 Hz), 7.63 (dd, 1H, J=2.6, 8.6 Hz), 6.70 (d, 1H, J=8.8 Hz), 4.42 (t, 2H, J=5.8 Hz), 3.77-3.70 (m, 4H), 2.77 (t, 2H, J=6.0 Hz), 2.58-2.53 (m, 4H).

Step 2

The procedure of Step 2 of Example 20 was repeated except for using the compound obtained in Step 1 instead of 2-benzyloxy-6-bromopyridine and conducting the reaction at 180° C. instead of 120° C. to obtain tert-butyl 4-(6-(2-morpholinoethoxy)pyri din-3-yl)piperazine-1-carboxylate (yield: 66%).

¹H NMR (200 MHz, CDCl₃) δ 7.76 (d, 1H, J=3.0 Hz), 7.31-7.25 (m, 1H), 6.71 (d, 1H, J=9.0 Hz), 4.39 (t, 2H, J=5.6 Hz), 3.75-3.71 (m, 4H), 3.60-3.55 (m, 4H), 3.02-3.00 (m, 4H), 2.77 (t, 1H, J=5.8 Hz), 2.59-2.54 (m, 4H), 1.48 (s, 9H).

Step 3

The procedure of Step 3 of Example 5 was repeated except for using the compound obtained in Step 2 instead of text-butyl-4-(5-chlorobenzooxazol-2-yl)piperazine-1-carboxylate to quantitatively obtain 1-(6-(2-morpholinoethoxy)pyridin-3-yl)piperazine.

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 43%).

¹H NMR (300 MHz, CDCl₃) δ 7.96 (s, 1H), 7.79 (s, 1H), 7.49-7.46 (m, 1H), 7.39 (t, 1H, J=8.0 Hz), 6.81-6.71 (m, 2H), 6.15-6.08 (m, 2H), 5.15 (br s, 1H), 4.95 (d, 1H, J=14.7 Hz), 4.85 (d, 1H, J=14.7 Hz), 4.39 (t, 2H, J=6.0 Hz), 3.74 (t, 4H, J=4.7 Hz), 3.50 (br s, 4H), 3.02-2.89 (m, 3H), 2.77 (t, 2H, J=6.0 Hz), 2.58-2.48 (m, 6H), 0.96 (d, 3H, J=6.9 Hz); MS (EI) m/z C₂₇H₃₅F₂N₇O₃ calc. 543, found 543 (M⁺, 1), 319 (100), 114 (29).

### EXAMPLE 35

### Preparation of (2R,3R)-2-(2,4-difluorophenyl)-3-(4-(6-(thiophen-2-ylmethoxy)pyridin-3-yl)piperazin-1-yl)-1-(1H-1,2,4-triazol-1-yl)butan-2-ol

Step 1

The procedure of Step 1 of Example 20 was repeated except for using 2,5-dibromopyridine instead of 2,6-dibromopyridine and thiophen-2-yl-methanol instead of benzylalcohol to obtain 2-((thiophen-2-yl)methoxy)-5-bromopyridine (yield: 96%).

¹H NMR (200 MHz, CDCl₃) δ 8.24 (d, 1H, J=2.6 Hz), 7.63-6.69 (m, 1H), 7.31 (d, 1H, J=5.2 Hz), 7.17-7.15 (m, 1H), 7.00 (dd, 1H, J=3.6, 4.8 Hz), 6.69 (d, 1H, J=9.0 Hz), 5.52 (s, 2H).

Step 2

The procedure of Step 2 of Example 20 was repeated except for using the compound obtained in Step 1 instead of 2-(benzyloxy)-6-bromopyridine and piperazine instead of tert-butyl piperazine-1-carboxylate to obtain 1-(6-(thiophen-2-ylmethoxy)pyridin-3-yl)piperazine (yield: 36%).

¹H NMR (200 MHz, CDCl₃) δ 7.81 (d, 1H, J=2.8 Hz), 7.32-7.27 (m, 2H), 7.13-7.12 (m, 1H), 7.02-6.96 (m, 1H), 6.72 (dd, 1H, J=2.0, 9.0 Hz), 5.49 (s, 2H), 3.05-3.04 (m, 8H).

Step 3

The procedure of Step 2 of Example 1 was repeated except for using the compound obtained in Step 2 instead of 2-(piperazin-1-yl)benzooxazole and propionitrilenitrile instead of acetonitrile to obtain the title compound (yield: 47%).

¹H NMR (300 MHz, CDCl₃) δ 7.97 (s, 1H), 7.79 (s, 1H), 7.50-7.47 (m, 1H), 7.40 (t, 1H, J=8.0 Hz), 7.28-7.26 (m, 1H), 7.11 (d, 1H, J=3.3 Hz), 6.99-6.96 (m, 1H), 6.81-6.74 (m, 2H), 6.17-6.10 (m, 2H), 5.49 (s, 2H), 5.21 (br s, 1H), 4.96 (d, 1H, J=14.7 Hz), 4.86 (d, 1H, J=14.7 Hz), 3.55 (br s, 4H), 3.05-2.91 (m, 3H), 2.57-2.50 (m, 2H), 0.97 (d, 3H, J=6.9 Hz); MS (EI) m/z C₂₆H₂₈F₂N₆O₂S calc. 526, found 526 (M⁺, 3), 148 (60), 96 (100).

### EXAMPLE 36

### Preparation of (2R,3R)-2-(2,4-difluorophenyl)-3-(4-(5-(methyl(3-morpholinopropyl)amino)pyridin-2-yl)piperazin-1-yl)-1-(1H-1,2,4-triazol-1-yl)butan-2-ol

Step 1

The procedure of Step 1 of Example 27 was repeated except for using 2,5-dibromopyridine instead of 2,6-dibromopyridine to obtain 5-bromo-N-(3-morpholinopropyl)pyridine-2-amine (yield: 64%).

¹H NMR (200 MHz, CDCl₃) δ 8.09 (d, 1H, J=2.4 Hz), 7.44 (dd, 1H, J=2.4, 8.8 Hz), 6.28 (d, 1H, J=9.0 Hz), 5.40 (br s, 1H), 3.73 (t, 4H, J=4.6 Hz), 3.34 (dd, 2H, J=6.6, 12.0 Hz), 2.51-2.44 (m, 6H), 1.78 (t, 2H, J=6.5 Hz).

Step 2

The procedure of Step 2 of Example 27 was repeated except for using the compound obtained in Step 1 instead of 6-bromo-N-(3-morpholinopropyl)pyridine-2-amine to obtain 5-bromo-N-methyl-N-(3-morpholinopropyl)pyridine-2-amine (yield: 68%).

¹H NMR (200 MHz, CDCl₃) δ 8.13 (d, 1H, J=2.4 Hz), 7.45 (dd, 1H, J=2.2, 9.0 Hz), 6.42 (d, 1H, J=9.0 Hz), 3.74-3.69 (m, 4H), 3.54 (t, 2H, J=7.2 Hz), 3.01 (s, 3H), 2.45-2.40 (m, 4H), 2.38 (t, 2H, J=7.2 Hz), 1.80-1.69 (m, 2H).

Step 3

The procedure of Step 3 of Example 27 was repeated except for using the compound obtained in Step 2 instead of 6-bromo-N-methyl-N-(3-morpholinopropyl)pyridine-2-amine to obtain tert-butyl 4-(6-(methyl(3-morpholinopropyl)amino)pyridin-3-yl)piperazine-1-carboxylate (yield: 66%).

¹H NMR (200 MHz, CDCl₃) δ 7.87 (d, 1H, J=2.8 Hz), 7.19 (dd, 1H, J=3.2, 9.4 Hz), 6.51 (d, 1H, J=9.2 Hz), 3.78-3.66 (m, 4H), 3.59-3.48 (m, 6H), 3.04-2.91 (m, 5H), 2.46-2.33 (m, 6H), 1.84-1.69 (m, 4H), 1.48 (s, 9H).

Step 4

The procedure of Step 3 of Example 5 was repeated except for using the compound obtained in Step 3 instead of tert-butyl-4-(5-chlorobenzooxazol-2-yl)piperazine-1-carboxylate to quantitatively obtain N-methyl-N-(3-morpholinopropyl)-5-(piperazin-1-yl)pyridine-2-amine.

Step 5

The procedure of Step 2 of Example 1 was repeated except for using the compound obtained in Step 4 instead of 2-(piperazin-1-yl)benzooxazole and propionitrilenitrile instead of acetonitrile to obtain the title compound (yield: 40%).

¹H NMR (300 MHz, CDCl₃) δ 7.96 (s, 1H), 7.87 (d, J=3.0 Hz, 1H), 7.78 (s, 1H), 7.49-7.46 (m, 1H), 7.18 (dd, J=3.0, 9.0 Hz, 1H), 6.80-6.71 (m, 2H), 6.51 (d, J=9.0 Hz, 1H), 5.20 (br s, 1H), 4.93 (d, J=14.4 Hz, 1H), 4.84 (d, J=14.4 Hz, 1H), 3.73-3.70 (m, 4H), 3.54-3.48 (m, 2H), 3.04-2.95 (m, 10H), 2.62-2.59 (m, 2H), 2.45-2.35 (m, 6H), 1.80-1.75 (m, 2H), 0.99 (d, J=6.9 Hz, 3H); MS (EI) m/z C₂₉H₄₀F₂N₈O₂ calc. 570, found 570 (M⁺, 39), 346 (100), 259 (97), 162 (23).

### EXAMPLE 37

### Preparation of (2R,3R)-2-(2,4-difluorophenyl)-3-(4-(6-(methyl(2-(thiophen-2-yl)ethyl)amino)pyridin-2-yl)piperazin-1-yl)-1-(1H-1,2,4-triazol-1-yl)butan-2-ol

Step 1

2,5-dibromopyridine (0.505 g, 2.13 mmol) and 2-(thiophen-2-yl)ethaneamine (0.542 g, 4.26 mmol) were heated at 150° C. for 4 hrs, and cooled to room temperature, followed by subjecting the mixture to silica gel column chromatography (n-hexane:ethyl acetate=9:1) to obtain 5-bromo-N-(2-(thiophen-2-yl)ethyl)pyridine-2-amine (yield: 80%).

¹H NMR (200 MHz, CDCl₃) δ 8.12 (d, 1H, J=2.2 Hz), 7.46 (dd, 1H, J=2.6, 8.8 Hz), 7.22 (dd, 1H, J=1.2, 4.8 Hz), 6.97-6.93 (m, 1H), 6.86-6.83 (m, 1H), 6.27 (d, 1H, J=9.4 Hz), 4.62 (br s, 1H), 3.57 (q, 2H, J=6.5 Hz), 3.12 (t, 2H, J=6.5 Hz).

Step 2

The procedure of Step 2 of Example 27 was repeated except for using the compound obtained in Step 1 instead of 2-bromo-6-(3-morpholinopropylamino)pyridine to obtain 5-bromo-N-methyl-N-(2-(thiophen-2-yl)ethyl)pyridine-2-amine (yield: 87%).

¹H NMR (200 MHz, CDCl₃) δ 8.17 (d, 1H, J=2.4 Hz), 7.51-7.45 (m, 1H), 7.14 (dd, 1H, J=1.0, 5.2 Hz), 6.93 (t, 1H, J=3.4 Hz), 6.82-6.80 (m, 1H), 6.35 (d, 1H, J=9.8 Hz), 3.78 (t, 2H, J=7.0 Hz), 3.09 (t, 2H, J=7.4 Hz), 2.96 (s, 3H).

Step 3

The procedure of Step 3 of Example 27 was repeated except for using the compound obtained in Step 2 instead of 6-bromo-N-methyl-N-(3-morpholinopropyl)pyridine-2-amine to obtain N-methyl-N-(2-(thiophen-2-yl)ethyl)-5-(piperazin-1-yl)pyridine-2-amine (yield: 59%).

¹H NMR (200 MHz, CDCl₃) δ 7.93 (dd, 1H, J=0.6, 2.8 Hz), 7.24-7.12 (m, 2H), 6.95-6.90 (m; 1H), 6.84-6.81 (m, 1H), 6.47 (dd, 1H, J=0.6, 9.2 Hz), 3.80-3.73 (m, 2H), 3.31 (br s, 1H), 3.13-3.03 (m, 10H), 2.98 (s, 3H).

Step 4

The procedure of Step 2 of Example 1 was repeated except for using the compound obtained in Step 3 instead of 2-(piperazin-1-yl)benzooxazole and propionitrilenitrile instead of acetonitrile to obtain the title compound (yield: 50%).

¹H NMR (300 MHz, CDCl₃) δ 7.96 (s, 1H), 7.91 (d, J=3.0 Hz, 1H), 7.78 (s, 1H), 7.52-7.40 (m, 1H), 7.20 (dd, 1H, J=3.0, 9.0 Hz), 7.13 (dd, 1H, J=1.1, 5.3 Hz), 6.94-6.91 (m, 1H), 6.83-6.75 (m, 3H), 6.47 (d, J=9.3 Hz, 1H), 5.29 (br s, 1H), 4.93 (d, 1H, J=14.4 Hz), 4.84 (d, 1H, J=15.0 Hz), 3.79-3.74 (m, 2H), 3.12-2.94 (m, 12H), 2.62-2.56 (m, 2H), 0.99 (d, J=6.9 Hz, 3H); MS (EI) m/z C₂₈H₃₃F₂N₇OS calc. 553, found 553 (M⁺, 32), 456 (29), 329 (100), 84 (59).

### EXAMPLE 38

### Preparation of (2R,3R)-2-(2,4-difluorophenyl)-3-(4-(pyridin-2-yl)-1,4-diazepan-1-yl)-1-(1H-1,2,4-triazol-1-yl)butan-2-ol

Step 1

1.58 g (10 mmol) of 2-bromopyridine and 2.5 g (25 mmol, 2.5 eq) of homopiperazine were placed in a 5 mL microwave reactor dried with nitrogen gas, and the reactor was kept at 120° C. for 20 mins. After adding water thereto, the reaction mixture was extracted more than 3 times with ethyl acetate, and the formed organic layer was washed with a saturated NaCl solution, dried over anhydrous magnesium sulfate, and concentrated under a reduced pressure. The resulting residue was subjected to silica gel column chromatography (dichloromethane:methanol=9:1) to obtain 1-(pyridin-2-yl)-1,4-diazepane (yield: 62%).

¹H NMR (200 MHz, CDCl₃) δ 8.17-8.13 (m, 1H), 7.53-7.45 (m, 1H), 6.63 (dd, 1H, J=5.0, 7.4 Hz), 6.53 (d, 1H, J=8.2 Hz), 4.12 (t, 2H, J=4.7 Hz), 3.75 (t, 2H, J=6.5 Hz), 3.38 (t, 2H, J=4.9 Hz), 3.23 (t, 2H, J=5.5 Hz), 2.39 (t, 2H, J=5.7 Hz).

Step 2

The procedure of Step 2 of Example 1 was repeated except for using the compound obtained in Step 1 instead of 2-(piperazin-1-yl)benzooxazole and propionitrilenitrile instead of acetonitrile to obtain the title compound (yield: 36%).

¹H NMR (300 MHz, CDCl₃) δ 8.14-8.12 (m, 1H), 7.89 (s, 1H), 7.75 (s, 1H), 7.48-7.38 (m, 2H), 6.79-6.67 (m, 2H), 6.52-6.47 (m, 2H), 5.30 (br s, 1H), 4.72-4.62 (m, 2H), 3.83-3.59 (m, 4H), 3.05-2.93 (m, 3H), 2.74-2.72 (m, 1H), 2.47-2.45 (m, 1H), 1.99-1.93 (m, 2H), 0.93 (d, J=6.9 Hz, 3H); MS (EI) m/z C₂₂H₂₆F₂N₆O calc. 428, found 429 (M⁺+1, 1), 306 (6), 205 (100), 147 (26).

### EXAMPLE 39

### Preparation of 6-(4-((2R,3R)-3-(2,4-difluorophenyl)-3-hydroxy-4-(1H-1,2,4-triazol-1-yl)butan-2-yl)-1,4-diazepan-1-yl)pyridine-3-carbonitrile

Step 1

The procedure of Step 1 of Example 17 was repeated except for using homopiperazine instead of piperazine and conducting the reaction at 180° C. to obtain 5-(1,4-diazepan-1-yl)picolinonitrile (yield: 50%).

¹H NMR (200 MHz, CDCl₃) δ 8.15 (d, 1H, J=3.2 Hz), 7.47 (d, 1H, J=9.0 Hz), 6.91 (dd, 1H, J=2.8, 8.8 Hz), 3.69-3.48 (m, 4H), 3.10-2.90 (m, 2H), 2.88-2.85 (m, 2H), 1.97-1.92 (m, 2H).

Step 2

The procedure of Step 2 of Example 1 was repeated except for using the compound obtained in Step 1 instead of 2-(piperazin-1-yl)benzooxazole and propionitrilenitrile instead of acetonitrile to obtain the title compound (yield: 45%).

¹H NMR (300 MHz, CDCl₃) δ 8.16 (d, J=3.0 Hz, 1H), 7.85 (s, 1H), 7.77 (s, 1H), 7.45 (d, J=8.7 Hz, 1H), 7.38 (dd, J=6.6, 9.0 Hz, 1H), 6.92 (dd, J=3.0, 8.7 Hz, 1H), 6.77-6.67 (m, 2H), 4.98 (br s, 1H), 4.80-4.69 (m, 2H), 3.71-3.58 (m, 4H), 3.28 (br s, 1H), 3.11 (q, J=6.9 Hz, 2H), 2.78-2.75 (m, 1H), 2.52 (br s, 1H), 1.92 (br s, 2H), 0.89 (d, J=6.6 Hz, 3H); MS (EI) m/z C₂₃H₂₅F₂N₇O calc. 453, found 454 (M⁺+1, 1), 229 (100), 141 (12).

### EXAMPLE 40

### Preparation of (2R,3R)-2-(2,4-difluorophenyl)-3-(4-(quinolin-2-yl)-1,4-diazepan-1-yl)-1-(1H-1,2,4-triazol-1-yl)butan-2-ol

Step 1

The procedure of Step 1 of Example 19 was repeated except for using homopiperazine instead of piperazine and conducting the reaction for 18 hrs to obtain 2-(1,4-diazepan-1-yl)quinoline (yield: 47%).

¹H NMR (200 MHz, CDCl₃) δ 7.85 (d, 1H, J=9.4 Hz), 7.68-7.46 (m, 3H), 7.21 (t, 1H, J=8.0 Hz), 6.86 (d, 1H, J=9.0 Hz), 3.95-3.84 (m, 4H), 3.11 (t, 2H, J=5.6 Hz), 2.87 (t, 2H, J=5.8 Hz), 2.02-1.91 (m, 2H).

Step 2

The procedure of Step 2 of Example 1 was repeated except for using the compound obtained in Step 1 instead of 2-(piperazin-1-yl)benzooxazole to obtain the title compound (yield: 47%).

¹H NMR (300 MHz, CDCl₃) δ 7.82 (d, J=9.0 Hz, 1H), 7.67-7.65 (m, 2H), 7.54-7.38 (m, 4H), 7.20-7.17 (m, 1H), 6.90 (d, J=8.7 Hz, 1H), 6.71-6.63 (m, 2H), 5.03 (br s, 1H), 4.47 (s, 2H), 3.95-3.85 (m, 4H), 3.14 (br s, 1H), 3.03 (q, 2H, J=6.7 Hz), 2.84 (br s, 1H), 2.49 (br s, 1H), 1.99-1.88 (m, 2H), 0.89 (d, J=7.0 Hz, 3H); MS (EI) m/z 478 (M⁺, 1), 254 (100), 184 (19), 127 (20).

### EXAMPLE 41

### Preparation of (2R,3R)-2-(2,4-difluorophenyl)-3-(4-((pyridin-2-yl)methyl)piperazin-1-yl)-1-(1H-1,2,4-triazol-1-yl)butan-2-ol

Step 1

1.00 g (6.09 mmol, 1 eq) of 2-(chloroethyl)pyridine hydrochloride and 1.05 g (12.19 mmol, 2 eq) of piperazine were dissolved in distilled water in a dried round flask provided with nitrogen gas, followed by stirring the mixture at room temperature for 16 hrs. The reaction mixture was extracted with ethyl acetate, and the water layer was mixed with NaOH and extracted with dichloromethane. The formed organic layer was dried over anhydrous magnesium sulfate and concentrated under a reduced pressure, and the resulting residue was subjected to silica gel column chromatography (dichloromethane:methanol=4:1) to obtain 1-((pyridin-2-yl)methyl)piperazine (yield: 70%).

¹H NMR (200 MHz, CDCl₃) δ 8.56 (d, 1H, J=2.8 Hz), 7.64 (dt, 1H, J=1.7, 7.6 Hz), 7.40 (d, 1H, J=7.8 Hz), 7.16 (t, 1H, J=5.0 Hz), 3.67 (s, 2H), 2.53 (br s, 8H).

Step 2

The procedure of Step 2 of Example 1 was repeated except for using the compound obtained in Step 1 instead of 2-(piperazin-1-yl)benzooxazole to obtain the title compound (yield: 28%).

¹H NMR (300 MHz, CDCl₃) δ 8.56 (d, 1H, J=4.8 Hz), 8.00 (s, 1H), 7.78 (s, 1H), 7.64 (t, 1H, J=8.1 Hz), 7.49-7.44 (m, 1H), 7.37 (d, 1H, J=7.8 Hz), 7.18-7.14 (m, 1H), 6.80-6.69 (m, 2H), 4.88 (d, 1H, J=14.4 Hz), 4.78 (d, 1H, J=14.1 Hz), 3.65 (s, 2H), 2.88 (q, 1H, J=6.9 Hz), 2.78-2.73 (m, 2H), 2.53-2.42 (m, 6H), 0.98 (d, 3H, J=6.9 Hz); MS (EI) m/z C₂₂H₂₆F₂N₆O calc. 428, found 429 (M⁺+1, 1), 361 (17), 204 (63), 100 (100).

### EXAMPLE 42

### Preparation of (2R,3R)-2-(2,4-difluorophenyl)-3-(4-((pyridin-4-yl)methyl)piperazin-1-yl)-1-(1H-1,2,4-triazol-1-yl)butan-2-ol

Step 1

The procedure of Step 1 of Example 41 was repeated except for using 4-(chloroethyl)pyridine hydrochloride instead of 2-(chloroethyl)pyridine hydrochloride to obtain 1-((pyridin-4-yl)methyl)piperazine (yield: 71%).

¹H NMR (200 MHz, CDCl₃) δ 8.53 (dd, 2H, J=1.7, 4.4 Hz), 7.27 (t, 2H, J=2.4 Hz), 3.48 (s, 2H), 2.61-2.08 (m, 8H).

Step 2

The procedure of Step 2 of Example 1 was repeated except for using the compound obtained in Step 1 instead of 2-(piperazin-1-yl)benzooxazole and propionitrilenitrile instead of acetonitrile to obtain the title compound (yield: 40%).

¹H NMR (300 MHz, CDCl₃) δ 8.53 (d, J=5.7 Hz, 2H), 7.97 (s, 1H), 7.77 (s, 1H), 7.45 (dd, J=6.5, 8.9 Hz, 1H), 7.27-7.24 (m, 2H), 6.79-6.68 (m, 2H), 5.15 (br s, 1H), 4.88 (d, J=14.7 Hz, 1H), 4.79 (d, J=14.7 Hz, 1H), 3.49 (s, 2H), 2.92 (q, J=6.9 Hz, 1H), 2.79 (t, J=7.0 Hz, 2H), 2.46-2.43 (m, 6H), 0.97 (d, J=6.9 Hz, 3H).

### EXAMPLE 43

### Preparation of (2R,3R)-2-(2,4-difluorophenyl)-3-(4-((furan-2-yl)methyl)piperazin-1-yl)-1-(1H-1,2,4-triazol-1-yl)butan-2-ol

Step 1

0.5 g (5.2 mmol) of 2-furaldehyde was dissolved in acetic acid in a dried round flask provided with nitrogen gas, 2.24 g (26 mmol) of piperazine was added thereto, and 0.4 g (6.36 mmol, 1.2 eq) of sodium cyanoborohydride was slowly added thereto at 0° C. After heating to room temperature, the reaction mixture was kept for 6 hrs, and concentrated under a reduced pressure to remove the solvent. The resulting solution was mixed with NaOH and extracted with dichloromethane. The formed organic layer was dried over anhydrous magnesium sulfate, and concentrated under a reduced pressure. The resulting residue was subjected to silica gel column chromatography (dichloromethane:methanol=4:1) to obtain 1-((furan-2-yl)methyl)piperazine (yield: 58%).

¹H NMR (200 MHz, CDCl₃) δ 7.39-7.37 (1H, m), 6.32-6.30 (1H, m), 6.20 (1H, d, J=3.0 Hz), 3.53 (2H, s), 2.94-2.90 (5H, m), 2.48-2.44 (4H, m); C₉H₁₄N₂O m/z 166.11 MS (EI) m/z 166 (M⁺, 9), 81 (100), 56 (85).

Step 2

The procedure of Step 2 of Example 1 was repeated except for using the compound obtained in Step 1 instead of 2-(piperazin-1-yl)benzooxazole to obtain the title compound (yield: 28%).

¹H NMR (300 MHz, CDCl₃) δ 7.98 (s, 1H), 7.78 (s, 1H), 7.48-7.45 (m, 1H), 7.38-7.37 (m, 1H), 6.79-6.69 (m, 2H), 6.31 (t, 1H, J=2.3 Hz), 6.20 (d, 1H, J=3.0 Hz), 4.86 (d, 1H, J=14.7 Hz), 4.77 (d, 1H, J=14.7), 3.53 (s, 2H), 2.89 (q, 1H, J=7.2 Hz), 2.79-2.75 (m, 2H), 2.47-2.43 (m 6H), 0.96 (d, 3H, J=7.1 Hz); MS (EI) m/z C₂₁H₂₅F₂N₅O₂ calc. 417, found 418 (M⁺+1, 1), 224 (87), 215 (100).

### EXAMPLE 44

### Preparation of (2R,3R)-2-(2,4-difluorophenyl)-3-(4-((thiophen-2-yl)methyl)piperazin-1-yl)-1-(1H-1,2,4-triazol-1-yl)butan-2-ol

Step 1

The procedure of Step 1 of Example 43 was repeated except for using thiophene-2-carboxaldehyde instead of 2-furaldehyde to obtain 1-((thiophen-2-yl)methyl)piperazine (yield: 49%).

¹H NMR (200 MHz, CDCl₃) δ 7.24-7.21 (m, 1H), 6.96-6.91 (m, 2H), 3.7 (s, 2H), 2.95-2.90 (m, 2H), 2.50 (br s, 7H).

Step 2

The procedure of Step 2 of Example 1 was repeated except for using the compound obtained in Step 1 instead of 2-(piperazin-1-yl)benzooxazole to obtain the title compound (yield: 28%).

¹H NMR (300 MHz, CDCl₃) δ 7.98 (s, 1H), 7.77 (s, 1H), 7.45 (dd, 1H, J=6.8, 9.3 Hz), 7.24-7.22 (m, 1H), 6.95-6.90 (m, 2H), 6.79-6.69 (m, 2H), 4.86 (d, 1H, J=14.4 Hz), 4.77 (d, 1H, J=14.4 Hz), 3.72 (s, 2H), 2.89 (q, 1H, J=7.0 Hz), 2.78-2.72 (m, 2H), 2.50-2.41 (m, 6H), 0.97 (d, 3H, J=7.0 Hz).

### EXAMPLE 45

### Preparation of (4-((2R,3R)-3-(2,4-difluorophenyl)-3-hydroxy-4-(1H-1,2,4-triazol-1-yl)butan-2-yl)piperazin-1-yl)(3-phenyl-1,2,4-oxadiazol-5-yl)methanone

Step 1

2.0 g (19.39 mmol, 1 eq) of benzonitrile, 4.0 g (58.18 mmol, 3 eq.) of hydroxyamine hydrochloride and 8.08 g (58.18 mmol, 3 eq.) of potassium carbonate were dissolved in ethanol in a dried round flask provided with nitrogen gas, followed refluxing the mixture with stirring for 5 hrs. After cooling to room temperature, the reaction mixture was concentrated under a reduced pressure, 20 mL of pyridine and 1.89 mL (16.9 mmol, 1.5 eq.) of ethylchlorooxoacetate were slowly added thereto at 0° C., and the resulting mixture was kept at 40° C. for 2 hrs. The resulting solution was concentrated under a reduced pressure, distilled water was added thereto, and the resulting mixture was extracted more than 3 times with ethyl acetate. The formed organic layer was washed with a saturated NaCl solution, dried over anhydrous magnesium sulfate, and concentrated under a reduced pressure. The resulting residue was subjected to silica gel column chromatography (n-hexane:ethyl acetate=9:1) to obtain ethyl 3-phenyl-1,2,4-oxadiazol-5-carboxylate (yield: 30%).

¹H NMR (200 MHz, CDCl₃) δ 8.18-8.13 (m, 2H), 7.56-7.46 (m, 3H), 4.58 (q, 2H, J=7.1 Hz), 1.50 (t, 3H, J=7.2 Hz).

Step 2

0.05 g (0.16 mmol) of the compound obtained in Step 1 of Example 14 and 0.04 g (0.19 mmol) of the compound obtained in Step 1 were placed in a dried 5 ml microwave reactor provided with nitrogen gas, followed by keeping the reactor at 120° C. for 10 mins. After adding distilled water thereto, the reaction mixture was extracted more than 3 times with ethyl acetate. The formed organic layer was washed with a saturated NaCl solution, dried over anhydrous magnesium sulfate, and concentrated under a reduced pressure. The resulting residue was subjected to silica gel column chromatography (dichloromethane:methanol=19:1) to obtain the title compound (yield: 65%).

¹H NMR (300 MHz, CDCl₃) δ 8.12 (dd, 2H, J=1.7, 7.7 Hz), 7.89 (s, 1H), 7.78 (s, 1H), 7.56-7.41 (m, 4H), 6.78-6.69 (m, 2H), 4.94 (br s, 3H), 3.88-3.81 (m, 4H), 3.14-3.07 (m, 3H), 2.67-2.57 (m, 2H), 0.91 (d, 3H, J=6.6 Hz).

### EXAMPLE 46

### Preparation of (3-(4-bromophenyl)-1,2,4-oxadiazol-5-yl)(4-((2R,3R)-3-(2,4-difluorophenyl)-3-hydroxy-4-(1H-1,2,4-triazol-1-yl)butan-2-yl)piperazin-1-yl)methanone

Step 1

The procedure of Step 1 of Example 45 was repeated except for using 4-bromobenzonitrile instead of benzonitrile to obtain 3-(4-bromophenyl)-1,2,4-oxadiazol-5-carboxylate (yield: 53%).

¹H NMR (200 MHz, CDCl₃) δ 8.03 (d, 2H, J=8.4 Hz), 7.66 (d, 2H, J=8.6 Hz), 4.37 (q, 2H, J=7.1 Hz), 1.42 (t, 3H, J=7.2 Hz); MS (EI) m/z C₁₁H₉BrN₂O₃ calc. 295, found 295 (M⁺, 57), 197 (94), 90 (82), 75 (100).

Step 2

The procedure of Step 2 of Example 45 was repeated except for using the compound obtained in Step 1 instead of ethyl 3-phenyl-1,2,4-oxadiazol-5-carboxylate to obtain the title compound (yield: 53%).

¹H NMR (300 MHz, CDCl₃) δ 8.00-7.91 (m, 2H), 7.89 (s, 1H), 7.78 (s, 1H), 7.66 (dd, 2H, J=2.0, 8.8 Hz), 7.43-7.37 (m, 1H), 6.78-6.69 (m, 2H), 4.99-4.94 (m, 3H), 3.86-3.73 (m, 4H), 3.14-3.07 (m, 3H), 2.67-2.57 (m, 2H), 0.90 (d, 3H, J=6.9 Hz)

### EXAMPLE 47

### Preparation of (4-((2R,3R)-3-(2,4-difluorophenyl)-3-hydroxy-4-(1H-1,2,4-triazol-1-yl)butan-2-yl)piperazin-1-yl)(3-p-tolyl-1,2,4-oxadiazol-5-yl)methanone

Step 1

The procedure of Step 1 of Example 45 was repeated except for using 4-methylbenzonitrile instead of benzonitrile to obtain ethyl 3-(4-methylphenyl)-1,2,4-oxadiazol-5-carboxylate (yield: 30%).

¹H NMR (200 MHz, CDCl₃) δ 8.03 (dd, 2H, J=1.8, 6.6 Hz), 7.30 (d, 2H, J=7.8 Hz), 4.58 (q, 2H, J=7.2 Hz), 2.43 (s, 3H), 1.49 (t, 3H, J=7.4 Hz).

Step 2

The procedure of Step 2 of Example 45 was repeated except for using the compound obtained in Step 1 instead of ethyl 3-phenyl-1,2,4-oxadiazol-5-carboxylate to obtain the title compound (yield: 64%).

¹H NMR (300 MHz, CDCl₃) δ 8.00 (d, 2H, J=8.2 Hz), 7.89 (s, 1H), 7.78 (s, 1H), 7.43-7.38 (m, 1H), 7.32-7.27 (m, 2H), 6.79-6.68 (m, 2H), 4.97-4.89 (m, 3H), 3.88-3.73 (m, 4H), 3.13-3.06 (m, 3H), 2.66-2.50 (m, 2H), 2.43 (s, 3H), 0.91 (d, 3H, J=6.6 Hz).

### EXAMPLE 48

### Preparation of (3-(4-chlorophenyl-1,2,4-oxadiazol-5-yl)(4-((2R,3R)-3-(2,4-difluorophenyl)-3-hydroxy-4-(1H-1,2,4-triazol-1-yl)butan-2-yl)piperazin-1-yl)methanone

Step 1

The procedure of Step 1 of Example 45 was repeated except for using 4-chlorobenzonitrile instead of benzonitrile to obtain ethyl 3-(4-chlorophenyl)-1,2,4-oxadiazol-5-carboxylate (yield: 59%).

¹H NMR (200 MHz, CDCl₃) δ 8.10 (d, 2H, J=9.0 Hz), 7.49 (d, 2H, J=8.8 Hz), 4.58 (q, 2H, J=7.1 Hz), 1.53 (t, 3H, J=7.2 Hz).

Step 2

The procedure of Step 2 of Example 45 was repeated except for using the compound obtained in Step 1 instead of ethyl 3-phenyl-1,2,4-oxadiazol-5-carboxylate to obtain the title compound (yield: 47%).

¹H NMR (300 MHz, CDCl₃) δ 8.06 (d, 21-1, J=8.7 Hz), 7.89 (s, 1H), 7.78 (s, 1H), 7.51-7.41 (m, 3H), 6.78-6.69 (m, 2H), 4.99-4.89 (m, 3H), 3.87-3.80 (m, 4H), 3.14-3.07 (m, 3H), 2.67-2.57 (m, 2H), 0.91 (d, 3H, J=6.6 Hz).

### EXAMPLE 49

### Preparation of (4-((2R,3R)-3-(2,4-difluorophenyl)-3-hydroxy-4-(1H-1,2,4-triazol-1-yl)butan-2-yl)piperazin-1-yl)(3-(4-methoxyphenyl)-1,2,4-oxadiazol-5-yl)methanone

Step 1

The procedure of Step 1 of Example 45 was repeated except for using 4-methoxybenzonitrile instead of benzonitrile to obtain ethyl 3-(4-methoxyphenyl)-1,2,4-oxadiazol-5-carboxylate (yield: 28%).

¹H NMR (200 MHz, CDCl₃) δ 8.09 (d, 2H, J=8.6 Hz), 7.59 (d, 2H, J=8.8 Hz), 4.57 (q, 2H, J=7.1 Hz), 3.87 (s, 3H), 1.46 (t, 3H, J=7.0 Hz).

Step 2

The procedure of Step 2 of Example 45 was repeated except for using the compound obtained in Step 1 instead of ethyl 3-phenyl-1,2,4-oxadiazol-5-carboxylate to obtain the title compound (yield: 47%).

¹H NMR (300 MHz, CDCl₃) δ 8.05 (dd, 2H, J=2.0, 6.6 Hz), 7.89 (s, 1H), 7.79 (s, 1H), 7.43-7.38 (m, 1H), 7.01 (dd, 2H, J=1.8, 6.9 Hz), 6.79-6.69 (m, 2H), 4.90 (br s, 3H), 3.91-3.80 (m, 7H), 3.13-3.06 (m, 3H), 2.66-2.56 (m, 2H), 0.91 (d, 3H, J=6.6 Hz).

### EXAMPLE 50

### Preparation of (4-((2R,3R)-3-(2,4-difluorophenyl)-3-hydroxy-4-(1H-1,2,4-triazol-1-yl)butan-2-yl)piperazin-1-yl)(3-phenyl-1,2,4-thiadiazol-5-yl)methanone

Step 1

1.0 g (8.25 mmol, 1 eq) of benzamide was dissolved in toluene in a 2-necked round flask provided with nitrogen gas, 0.83 mL (9.90 mmol, 1.2 eq.) of chlorocarbonylsulfenyl chloride was added thereon, and the mixture was refluxed with stirring for 3 hrs. After adding distilled water thereto, the reaction mixture was extracted more than 3 times with ethyl acetate, and the formed organic layer was washed with a saturated NaCl solution, and dried over anhydrous magnesium sulfate and concentrated under a reduced pressure. The resulting residue was subjected to silica gel column chromatography (n-hexane:ethyl acetate=19:1) to obtain 3-phenyl-5H-1,2,4-oxathiazol-5-one (yield: 87%).

¹H NMR (200 MHz, CDCl₃) δ 8.00-7.96 (2H, m), 7.62-7.29 (3H, m); MS (EI) m/z C₈H₅NO₂S calc. 179, found 179 (M⁺, 25), 105 (100).

Step 2

0.3 g (1.7 mmol, 1 eq) of the compound obtained in Step 1 was dissolved in n-dodecane in a 2-necked round flask provided with nitrogen gas, 0.66 g (6.8 mmol, 4 eq.) of ethyl cyanoformate was added thereto, and the mixture was kept at 130° C. for 24 hrs. After adding distilled water thereto, the reaction mixture was extracted more than 3 times with ethyl acetate, and the formed organic layer was washed with a saturated NaCl solution, dried over anhydrous magnesium sulfate and concentrated under a reduced pressure. The resulting residue was subjected to silica gel column chromatography (n-hexane:ethyl acetate=19:1) to obtain ethyl 3-phenyl-5H-1,2,4-oxathiazol-5-carboxylate (yield: 85%).

¹H NMR (200 MHz, CDCl₃) δ 8.39-8.35 (m, 2H), 7.54-7.48 (m, 3H), 4.55 (q, 2H, J=7.2 Hz), 1.51 (t, 3H, J=7.4 Hz); MS (EI) m/z C₁₁H₁₀N₂O₂S calc. 234, found 234 (M⁺, 34), 135 (100).

Step 3

The procedure of Step 2 of Example 45 was repeated except for using the compound obtained in Step 2 instead of ethyl 3-phenyl-1,2,4-oxadiazol-5-carboxylate to obtain the title compound (yield: 31%).

¹H NMR (500 MHz, CDCl₃) δ 8.30-8.27 (m, 2H), 7.91 (s, 1H), 7.80 (s, 1H), 7.52-7.49 (m, 3H), 7.44 (dt, 1H, J=6.6, 9.1 Hz), 6.79-6.71 (m, 2H), 5.00 (br s, 1H), 4.99 (d, 1H, J=14.6 Hz), 4.94 (d, 1H, J=15.5 Hz), 4.50 (br s, 2H), 3.85 (br s, 2H), 3.20 (br s, 1H), 3.11 (q, 2H, J=6.9 Hz), 2.64-2.63 (m, 2H), 0.92 (d, 3H, J=6.8 Hz).

### EXAMPLE 51

### Preparation of (3-(4-chlorophenyl)-1,2,4-thiadiazol-5-yl)(4-((2R,3R)-3-(2,4-difluorophenyl)-3-hydroxy-4-(1H-1,2,4-triazol-1-yl)butan-2-yl)piperazin-1-yl)methanone

Step 1

The procedure of Step 1 of Example 50 was repeated except for using 4-chlorobenzamide instead of benzamide to obtain 3-(4-chlorophenyl)-5H-1,2,4-oxathiazol-5-one (yield: 85%).

¹H NMR (200 MHz, CDCl₃) δ 7.92 (2H, d, J=7.6 Hz), 7.48 (2H, d, J=7.6 Hz); MS (EI) m/z C₈H₄ClNO₂S calc. 213, found 213 (M⁺, 21), 139 (100).

Step 2

The procedure of Step 2 of Example 50 was repeated except for using the compound obtained in Step 1 instead of 3-phenyl-5H-1,2,4-oxathiazol-5-one to obtain ethyl 3-(4-chlorophenyl)-5H-1,2,4-oxathiazol-5-carboxylate (yield: 70%).

¹H NMR (200 MHz, CDCl₃) δ 8.31 (d, 2H, J=8.0 Hz), 7.47 (d, 2H, J=8.4 Hz), 4.54 (q, 2H, J=7.4 Hz), 1.49 (t, 3H, J=7.2 Hz).

Step 3

The procedure of Step 2 of Example 45 was repeated except for using the compound obtained in Step 2 instead of ethyl 3-phenyl-1,2,4-oxadiazol-5-carboxylate to obtain the title compound (yield: 30%).

¹H NMR (300 MHz, CDCl₃) δ 8.22 (d, 2H, J=7.6 Hz), 7.90 (s, 1H), 7.80 (s, 1H), 7.49-7.39 (m, 3H), 6.80-6.69 (m, 2H), 5.02-4.91 (m, 3H), 4.55 (br s, 1H), 4.33 (br s, 1H), 3.83 (br s, 2H), 3.21 (br s, 1H), 3.11 (q, 2H, J=6.8 Hz), 2.65-2.62 (m, 2H), 0.91 (d, 3H, J=6.9 Hz).

### EXAMPLE 52

### Preparation of (3-(4-fluorophenyl)-1,2,4-thiadiazol-5-yl)(4-((2R,3R)-3-(2,4-difluorophenyl)-3-hydroxy-4-(1H-1,2,4-triazol-1-yl)butan-2-yl)piperazin-1-yl)methanone

Step 1

The procedure of Step 1 of Example 50 was repeated except for using 4-fluorobenzamide instead of benzamide to obtain 3-(4-fluorophenyl)-5H-1,2,4-oxathiazol-5-one (yield: 85%).

¹H NMR (200 MHz, CDCl₃) δ 8.03-7.96 (2H, m), 7.31-7.14 (2H, m); MS (EI) m/z 197 (M⁺+1, 19), 123 (100).

Step 2

The procedure of Step 2 of Example 50 was repeated except for using the compound obtained in Step 2 instead of 3-phenyl-5H-1,2,4-oxathiazol-5-one to obtain ethyl 3-(4-fluorophenyl)-5H-1,2,4-oxathiazol-5-carboxylate (yield: 70%).

¹H NMR (200 MHz, CDCl₃) δ 8.41-8.34 (m, 2H), 7.22-7.13 (m, 2H), 4.55 (q, 2H, J=7.1 Hz), 1.48 (t, 3H, J=7.1 Hz).

Step 3

The procedure of Step 2 of Example 45 was repeated except for using the compound obtained in Step 2 instead of ethyl 3-phenyl-1,2,4-oxadiazol-5-carboxylate to obtain the title compound (yield: 35%).

¹H NMR (300 MHz, CDCl₃) δ 8.30-8.25 (m, 2H), 7.90 (s, 1H), 7.79 (s, 1H), 7.44 (q, 1H, J=6.3 Hz), 7.18 (dt, 2H, J=1.9, 7.7 Hz), 6.77-6.70 (m, 2H), 5.02-4.90 (m, 3H), 4.55 (br s, 1H), 4.31 (br s, 1H), 3.94 (br s, 1H), 3.83 (br s, 1H), 3.21 (br s, 1H), 3.11 (q, 2H, J=6.7 Hz), 2.65-2.62 (m, 2H), 0.91 (d, 3H, J=6.3 Hz)

### EXAMPLE 53

### Preparation of (4-((2R,3R)-3-(2,4-difluorophenyl)-3-hydroxy-4-(1H-1,2,4-triazol-1-yl)butan-2-yl)piperazin-1-yl)(3-p-tolyl-1,2,4-thiadiazol-5-yl)methanone

Step 1

The procedure of Step 1 of Example 50 was repeated except for using 4-methylbenzamide instead of benzamide to obtain 3-(4-methylphenyl)-5H-1,2,4-oxathiazol-5-one (yield: 84%).

¹H NMR (200 MHz, CDCl₃) δ 7.88 (d, 2H, J=8.4 Hz), 7.29 (d, 2H, J=8.0 Hz), 2.43 (s, 3H).

Step 2

The procedure of Step 2 of Example 50 was repeated except for using the compound obtained in Step 1 instead of 3-phenyl-5H-1,2,4-oxathiazol-5-one to obtain ethyl 3-(4-methylphenyl)-5H-1,2,4-oxathiazol-5-carboxylate (yield: 91%).

¹H NMR (200 MHz, CDCl₃) δ 8.25 (d, 2H, J=8.0 Hz), 7.29 (d, 2H, J=8.6 Hz), 4.55 (q, 2H, J=7.2 Hz), 2.43 (s, 3H), 1.48 (t, 3H, J=6.8 Hz); MS (EI) m/z C₁₂H₁₂N₂O₂S calc. 248, found 248 (M⁺, 57), 149 (100).

Step 3

The procedure of Step 2 of Example 45 was repeated except for using the compound obtained in Step 2 instead of ethyl 3-phenyl-1,2,4-oxadiazol-5-carboxylate to obtain the title compound (yield: 52%).

¹H NMR (300 MHz, CDCl₃) δ 8.16 (d, 2H, J=8.1 Hz), 7.91 (s, 1H), 7.79 (s, 1H), 7.48-7.40 (m, 1H), 7.30 (d, 2H, J=8.1 Hz), 6.80-6.69 (m, 2H), 5.02-4.90 (m, 3H), 4.53 (br s, 1H), 4.35 (br s, 1H), 3.91 (br s, 1H), 3.84 (br s, 1H), 3.19-3.07 (m, 3H), 2.64-2.62 (m, 2H), 2.43 (s, 3H), 0.91 (d, 3H, J=6.9 Hz).

### EXAMPLE 54

### Preparation of (4-((2R,3R)-3-(2,4-difluorophenyl)-3-hydroxy-4-(1H-1,2,4-triazol-1-yl)butan-2-yl)piperazin-1-yl)(3-(4-methoxyphenyl)-1,2,4-thiadiazol-5-yl)methanone

Step 1

The procedure of Step 1 of Example 50 was repeated except for using 4-methoxybenzamide instead of benzamide to obtain 3-(4-methoxyphenyl)-5H-1,2,4-oxathiazol-5-one (yield: 84%).

¹H NMR (200 MHz, CDCl₃) δ 7.92 (d, 2H, J=9.2 Hz), 6.97 (d, 2H, J=9.0 Hz), 3.88 (s, 3H).

Step 2

The procedure of Step 2 of Example 50 was repeated except for using the compound obtained in Step 1 instead of 3-phenyl-5H-1,2,4-oxathiazol-5-one to obtain ethyl 3-(4-methoxyphenyl)-5H-1,2,4-oxathiazol-5-carboxylate (yield: 92%).

¹H NMR (200 MHz, CDCl₃) δ 8.30 (d, 2H, J=8.8 Hz), 7.98 (d, 2H, J=9.0 Hz), 4.55 (q, 2H, J=7.4 Hz), 3.88 (s, 3H), 1.49 (t, 3H, J=7.2 Hz); MS (EI) m/z C₁₂H₁₂N₂O₃S calc. 264, found 264 (M⁺, 100), 165 (88), 133 (95).

Step 3

The procedure of Step 2 of Example 45 was repeated except for using the compound obtained in Step 2 instead of ethyl 3-phenyl-1,2,4-oxadiazol-5-carboxylate to obtain the title compound (yield: 47%).

¹H NMR (300 MHz, CDCl₃) δ 8.22 (d, 2H, J=7.1 Hz), 7.91 (s, 1H), 7.79 (s, 1H), 7.45-7.42 (m, 1H), 7.00 (d, 2H, J=6.9 Hz), 6.80-6.70 (m, 2H), 5.02-4.90 (m, 3H), 4.54 (br s, 1H), 4.33 (br s, 1H), 3.96-3.82 (m, 5H), 3.19-3.07 (m, 3H), 2.64-2.62 (m, 2H), 0.91 (d, 3H, J=6.9 Hz).

### EXAMPLE 55

### Preparation of (4-((2R,3R)-3-(2,4-difluorophenyl)-3-hydroxy-4-(1H-1,2,4-triazol-1-yl)butan-2-yl)piperazin-1-yl)(5-benzoyl-imidazo[2,1,-b]-thiazol-2-yl)methanone

Step 1

1.0 g (13.1 mmol) of thiourea was dissolved in dichloromethane in a round flask provided with nitrogen gas, 5.25 ml (39.3 mmol) of DMF-DMA (N,N-dimethylformamidine dimethyl acetal) was added thereto, and the mixture was refluxed with stirring for 4 hrs. The reaction mixture was concentrated under a reduced pressure, and the resulting yellow solid was recrystallized with diethylether to obtain 1.84 g of N′,N″-thiocarbonylbis(N,N-dimethylformimidamide) (yield: 74%).

¹H NMR (200 MHz, DMSO-d₆) δ 8.70 (s, 2H), 3.16 (s, 6H), 3.01 (s, 6H).

Step 2

1.84 g (9.87 mmol) of the compound obtained in Step 1 was dissolved in dichloromethane in a dried flask provided with nitrogen gas, 1.09 mL (11.8 mmol, 1.2 eq) of methyl bromoacetate was slowly added dropwise thereto, and the mixture was stirred for 15 mins. After adding 2.8 mL (19.8 mmol, 2 eq) of triethylamine thereto, the reaction mixture was kept at room temperature for 20 hrs, distilled water was added thereto, and the resulting mixture was extracted more than 3 times with ethyl acetate. The formed organic layer was washed with a saturated NaCl solution, dried over anhydrous magnesium sulfate, and concentrated under a reduced pressure. The resulting residue was subjected to silica gel column chromatography (ethyl acetate:dichloromethane=9:1) to obtain 1.5 g of methyl 2-((dimethylamino)methyleneamino)thiazol-5-carboxylate (yield: 73%).

¹H NMR (200 MHz, CDCl₃) δ 8.35 (s, 1H), 7.98 (s, 1H), 3.85 (s, 3H), 3.14 (s, 3H), 3.11 (s, 3H).

Step 3

1.5 g (7.2 mmol) of the compound obtained in Step 2 was dissolved in THF in a dried flask provided with nitrogen gas, 2-bromo-1-phenylethanone (8.5 mmol) was added thereto, and the mixture was refluxed with stirring for 6 hrs. After cooling to room temperature, 2.0 mL (14.4 mmol) of triethylamine was added thereto, and the reaction mixture was kept at room temperature for 20 hrs. Distilled water was added thereto, followed by extracting the resulting mixture with ethyl acetate more than 3 times. The formed organic layer was washed with a saturated NaCl solution, dried over anhydrous magnesium sulfate, and concentrated under a reduced pressure. The resulting residue was subjected to silica gel column chromatography (ethyl acetate:dichloromethane=9:1) to obtain methyl 5-benzoylimidazo[2,1-b]thiazol-2-carboxylate (yield: 65%).

¹H NMR (300 MHz, CDCl₃) δ 9.09 (1H, s), 7.93 (1H, s), 7.85 (d, 2H, J=7.1 Hz), 7.60-7.55 (m, 1H), 7.48 (t, 2H, J=7.3 Hz), 3.92 (3H, s).

Step 4

5 mmol of the compound obtained in Step 3 and 10 mL of 1N NaOH were placed in a dried flask provided with nitrogen gas, followed by keeping the reactor at room temperature for 12 hrs. After adding distilled water thereto, the reaction mixture was extracted with ethyl acetate more than 3 times, and the formed organic layer was washed with a saturated NaCl solution, dried over anhydrous magnesium sulfate, and concentrated under a reduced pressure to quantitatively obtain 5-benzoylimidazo[2,1-b]thiazol-2-carboxylic acid.

Step 5

0.06 g (0.18 mmol) of the compound obtained in Step 1 of Example 14 was dissolved in dichloromethane in a dried flask provided with nitrogen gas, 50 mg (0.18 mmol) of the compound obtained in Step 4 and 0.22 mmol of N-(3-dimethylaminopropyl)-N′-ethylcarbodiimide hydrochloride were added thereto, followed by keeping the reactor at room temperature for 4 hrs. After adding distilled water thereto, the reaction mixture was extracted with ethyl acetate more than 3 times, and the formed organic layer was washed with a saturated NaCl solution, dried over anhydrous magnesium sulfate, and concentrated under a reduced pressure. The resulting residue was subjected to silica gel column chromatography (dichloromethane:methanol=19:1) to obtain the title compound (yield: 55%).

¹H NMR (300 MHz, CDCl₃) δ 8.72 (s, 1H), 7.97 (s, 1H), 7.92-7.89 (m, 3H), 7.79 (s, 1H), 7.64-7.52 (m, 3H), 7.44-7.40 (m, 1H), 6.77-6.72 (m, 2H), 5.00-4.90 (m, 3H), 3.86 (br s, 4H), 3.13-3.07 (m, 3H), 2.61-2.57 (m, 2H), 0.92 (d, 3H, J=6.9 Hz).

### EXAMPLE 56

### Preparation of (4-((2R,3R)-3-(2,4-difluorophenyl)-3-hydroxy-4-(1H-1,2,4-triazol-1-yl)butan-2-yl)piperazin-1-yl)(5-(4-methylbenzoyl)-imidazo[2,1-b]-thiazol-2-yl)methanone

Step 1

The procedure of Step 3 of Example 55 was repeated except for using ethyl 2-bromo-1-p-tolylethanone instead of 2-bromo-1-phenylethanone to obtain methyl 5-(4-methylbenzoyl)imidazo[2,1-b]thiazol-2-carboxylate (yield: 70%).

¹H NMR (300 MHz, CDCl₃) δ 9.07 (s, 1H), 7.92 (s, 1H), 7.75 (d, 2H, J=8.1 Hz), 7.27 (d, 2H, J=8.2 Hz), 3.91 (s, 3H), 2.40 (s, 3H).

Step 2

The procedure of Step 4 of Example 55 was repeated except for using the compound obtained in Step 1 instead of to methyl 5-benzoylimidazo[2,1-b]thiazol-2-carboxylate quantitatively obtain 5-(4-methylbenzoyl)imidazo[2,1-b]thiazol-2-carboxylic acid.

Step 3

The procedure of Step 5 of Example 55 was repeated except for using the compound obtained in Step 2 instead of 5-benzoylimidazo[2,1-b]thiazol-2-carboxylic acid to obtain the title compound (yield: 57%).

¹H NMR (300 MHz, CDCl₃) δ 8.71 (s, 1H), 7.97 (s, 1H), 7.91 (s, 1H), 7.83-7.79 (m, 3H), 7.47-7.33 (m, 3H), 6.78-6.69 (m, 2H), 4.99-4.90 (m, 3H), 3.86 (br s, 4H), 3.11-3.09 (m, 3H), 2.60-2.57 (m, 2H), 2.47 (s, 3H), 0.92 (d, 3H, J=6.7 Hz).

### EXAMPLE 57

### Preparation of (4-((2R,3R)-3-(2,4-difluorophenyl)-3-hydroxy-4-(1H-1,2,4-triazol-1-yl)butan-2-yl)piperazin-1-yl)(5-(4-methylbenzoyl)-imidazo[2,1-b]thiazol-2-yl)methanone

Step 1

The procedure of Step 3 of Example 55 was repeated except for using ethyl 2-bromo-1-(4-methoxyphenyl)ethanone instead of 2-bromo-1-phenylethanone to obtain methyl 5-(4-methoxybenzoyl)imidazo[2,1-b]thiazol-2-carboxylate (yield: 40%).

¹H NMR (300 MHz, CDCl₃) δ 9.09 (1H, s), 7.94 (1H, s), 7.91-7.88 (2H, m), 7.01-6.97 (2H, m), 3.93 (3H, s), 3.86 (3H, s).

Step 2

The procedure of Step 4 of Example 55 was repeated except for using the compound obtained in Step 1 instead of methyl 5-benzoyl imidazo[2,1-b]thiazol-2-carboxylate to obtain 5-(4-methoxybenzoyl)imidazo[2,1-b]thiazol-2-carboxylic acid.

Step 3

The procedure of Step 5 of Example 55 was repeated except for using the compound obtained in Step 2 instead of 5-benzoylimidazo[2,1-b]thiazol-2-carboxylic acid to obtain the title compound (yield: 50%).

¹H NMR (300 MHz, CDCl₃) δ 8.69 (s, 1H), 7.97-7.94 (m, 2H), 7.91 (d, 2H, J=3.3 Hz), 7.79 (s, 1H), 7.44-7.39 (m, 1H), 7.03 (d, 2H, J=8.7 Hz), 6.79-6.68 (m, 2H), 5.00-4.90 (m, 3H), 3.91-3.83 (m, 7H), 3.13-3.08 (m, 3H), 2.60-2.56 (m, 2H), 0.89 (d, 3H, J=6.6 Hz).

### TEST EXAMPLE 1

### Antifungal Activity In Vitro

In vitro antifungal activities of the inventive antifungal compounds were evaluated using test strains including Candida albican (ATCC 90873, 204276, 62342, 64124, 64550, 96901, MYA-573, MYA-574, MYA-575, MYA-576, MYA-1003) and Aspergillus fumigatus (ATCC 16424). Test samples and positive control samples were prepared by dissolving the inventive compounds and comparative compounds, i.e., amphotericin B, fluconazole and itraconazole, in DMSO, respectively, and each successively diluted with medium to obtain test and positive control solutions having test compound concentrations of 0.125 μg/ml to a maximum concentration not generating turbidity. Minimal inhibitory concentration (MIC₈₀) of each compound was determined as the lowest concentration of the test compounds required to reduce growth by 80% relative to a control strain not treated.

1) Test strain: Candida albican ATCC 90873, 204276, 62342, 64124, 64550, 96901, MYA-573, MYA-574, MYA-575, MYA-576, MYA-1003, and Aspergillus fumigatus ATCC 16424 were commercially obtained from The American Type Culture Collection (ATCC) and subcultured in CHEMON Co. Ltd. (amphotericin B was commercially purchased from Sigma, and fluconazole and itraconazole were prepared according to methods described in British Patent No. 2,099,818; and U.S. Pat. No. 4,267,179, respectively).

2) Medium and culture condition: Candida albican was cultured in Sabourad Dextrose Agar, YM Agar or Potato Dextrose Agar according to ATCC information at a temperature of 37° C., 35° C., 30° C. or 25° C. Aspergillus fumigatus was cultured in Malt Extract Agar or Potato Dextrose Agar at a temperature ranging from 24 to 27° C.

3) Preparation of test samples: the inventive compounds and comparative compounds were each diluted with DMSO to obtain 1 to 2 ml of a test sample in a concentration of 100-folds of the determining maximum concentration (256 μg/ml).

4) Preparation of broth

The test samples were each successively diluted with RPMI 1640 to obtain test solutions having concentrations of 0.25 to 256 mg/ml in a 12×75 mm sterilized disposable culture tube. The final concentration of DMSO was adjusted to 2% (v/v).

5) Preparation of strain solution and inoculation

Candida albican strains were each subcultured in Sabourad Dextrose Agar, YM Agar or Potato Dextrose Agar medium at 35° C. for 2˜3 days. In the case of yeasts, a single colony was taken from prominent colonies and suspended in 0.85% sterile physiological saline solution and the turbidity of the suspension was adjusted to 80˜82% at 530 nm, and then diluted 50-fold with RPMI 1640 medium to 1.0×10³˜5.0×10³ CFU/ml. The turbidity of fungi was adjusted to 80˜82% and the suspension was diluted 50-fold to 0.4102˜0.5104 CFU/ml. Aspergillus fumigatus strains were subcultured in Malt Extract Agar for 7˜10 days, and a single colony was taken from prominent colonies and suspended in 0.85% sterile physiological saline solution and the suspension was adjusted to 0.108 at 530 nm, and then diluted 1000-fold with RPMI 1640 medium to 0.4×10²˜5×10⁴ CFU/ml. The turbidity of fungi was adjusted to 80˜82% and the suspension was diluted 50-fold to 0.4102˜0.5104 CFU/ml.

The strain solutions thus obtained were each seeded into a sterilized 96-well microplate, 0.1 ml of each test solution were added thereto, and 10 ml of alamarblue was treated to each well (Biosource, #DAL1100). This procedure was repeated 2 times.

Minimal inhibitory concentration (MIC₈₀) of each compound was determined as the lowest concentration of the test compounds required to reduce growth by 80% relative to a control strain not treated. The results are shown in Table 2.

TABLE 2

| | | ATCC | | | | | | | | | | |
|---|---|---|---|---|---|---|---|---|---|---|---|---|
| Ex. | ATCC 16424 | 90873 | ATCC 96901 | MYA 1003 | 204276 | 62342 | 64124 | 64550 | MYA 573 | MYA 574 | MYA 575 | MYA 576 |
| 1 | 0.5 | 0.125 | 4 | 128 | 0.125 | 0.125 | 4 | 1 | 1 | 32 | 32 | 0.125 |
| 2 | 0.5 | 0.125 | 2 | 128 | 0.125 | 0.5 | 4 | 1 | 0.25 | 8 | 8 | 0.125 |
| 3 | 1 | 0.125 | 4 | >128 | 0.125 | 8 | 16 | 2 | 4 | 16 | 16 | 0.125 |
| 4 | 0.5 | 0.125 | 2 | >128 | | 0.125 | 8 | 0.5 | 0.125 | 0.25 | 0.125 | 0.125 |
| 5 | | 0.125 | 2 | >128 | | 0.125 | 2 | 0.5 | 0.25 | 0.5 | 0.125 | 0.125 |
| 6 | 0.125 | 0.125 | 2 | >128 | | 0.25 | 4 | 0.5 | 0.25 | 0.25 | 0.125 | 0.125 |
| 10 | | 0.125 | 4 | >128 | | 0.125 | 4 | 0.5 | 0.5 | 0.5 | 0.125 | 0.125 |
| 12 | 2 | 1 | 2 | 8 | | 8 | 4 | 2 | 2 | 4 | 1 | 0.25 |
| 13 | | 0.125 | 8 | 32 | | 0.25 | 2 | 0.5 | 0.25 | 16 | 0.125 | 0.125 |
| 15 | | 0.125 | 16 | >128 | | 0.25 | 8 | 2 | 0.5 | 1 | 0.125 | 0.125 |
| 16 | 16 | 0.125 | 16 | 64 | 0.125 | 1 | 8 | 1 | 0.5 | 32 | 32 | 0.125 |
| 17 | 8 | 0.125 | 8 | 32 | 0.125 | 2 | 16 | 1 | 0.25 | 64 | 64 | 0.125 |
| 18 | 0.125 | 0.125 | 8 | >128 | 0.125 | 4 | 16 | 2 | 1 | >128 | >128 | 0.125 |
| 19 | 0.125 | 0.125 | 0.5 | >128 | 0.125 | 0.25 | 4 | 0.5 | 0.25 | >128 | >128 | 0.125 |
| 20 | | 0.125 | 2 | >128 | | 1 | 8 | 1 | 0.5 | 1 | 0.25 | 0.25 |
| 21 | | 0.125 | >128 | >128 | | 8 | >128 | 8 | 8 | 128 | 0.25 | 0.25 |
| 22 | | 0.5 | >128 | >128 | | 128 | >128 | 64 | 32 | >128 | 0.5 | 0.5 |
| 23 | | 0.25 | >128 | >128 | | >128 | >128 | >128 | >128 | >128 | 0.5 | 0.5 |
| 24 | | 0.125 | >128 | >128 | | 8 | >128 | 2 | 4 | 8 | 0.5 | 0.25 |
| 25 | | 0.25 | >128 | >128 | | >128 | >128 | >128 | 2 | >128 | 1 | 0.5 |
| 26 | | 0.25 | >128 | >128 | | 4 | >128 | 8 | 8 | 16 | 0.5 | 0.5 |
| 27 | | 0.25 | 16 | 64 | | 2 | 32 | 4 | 4 | 8 | 1 | 0.5 |
| 28 | | 0.25 | >128 | >128 | | >128 | >128 | >128 | >128 | >128 | 1 | 1 |
| 29 | | 0.125 | >128 | >128 | | 0.5 | 4 | 1 | 1 | 1 | 0.25 | 0.125 |
| 30 | | 0.125 | 32 | >128 | | 1 | 32 | 1 | 1 | 4 | 0.125 | 0.125 |
| 31 | | 0.125 | 4 | >128 | | 1 | 64 | 1 | 2 | 4 | 0.25 | 0.25 |
| 32 | | 0.125 | 8 | >128 | | 1 | 4 | 1 | 1 | 4 | 0.5 | 0.125 |
| 33 | | 0.125 | 64 | >128 | | 2 | 64 | 4 | 2 | 4 | 0.125 | 0.125 |
| 34 | | 0.125 | 32 | 128 | | 0.5 | 32 | 2 | 2 | 2 | 0.125 | 0.125 |
| 35 | | 0.125 | >128 | >128 | | 0.5 | 8 | 1 | 0.5 | 1 | 0.125 | 0.125 |
| 36 | | 2 | 16 | 128 | | 4 | 128 | 16 | 8 | 8 | 2 | 4 |
| 37 | | 0.125 | >128 | >128 | | 1 | 32 | 4 | 1 | 2 | 0.5 | 0.5 |
| 38 | 0.125 | 0.125 | 32 | 64 | | 0.25 | 16 | 2 | 1 | 2 | 0.125 | 0.125 |
| 39 | | 0.125 | 32 | 16 | | 4 | 128 | 2 | 1 | 2 | 0.125 | 0.125 |
| 40 | | 0.125 | 16 | >128 | | 0.25 | 8 | 2 | 0.5 | 2 | 0.125 | 0.125 |
| 41 | 1 | 0.125 | 32 | >128 | 0.125 | | 64 | 16 | 2 | 64 | 64 | 0.125 |
| 42 | | 0.125 | 64 | 128 | | 0.5 | 64 | 4 | 1 | 4 | 0.5 | 0.125 |
| 43 | 64 | 0.125 | 64 | >128 | 0.125 | 4 | 64 | 16 | 4 | 128 | 128 | 0.25 |
| 44 | 0.5 | 0.125 | 64 | >128 | 0.125 | 4 | 32 | 4 | 2 | 32 | 32 | 0.125 |
| 45 | 0.125 | 0.125 | >128 | >128 | | | | | 1 | | | |
| 46 | 0.126 | 0.125 | >128 | >128 | | | | | >128 | | | |
| 47 | 0.25 | 0.125 | >128 | >128 | | | | | 0.25 | | | |
| 48 | 0.126 | 0.126 | >128 | >128 | | | | | 0.25 | | | |
| 49 | 0.5 | 0.125 | >128 | >128 | | | | | 1 | | | |
| 50 | 0.5 | 0.125 | >128 | >128 | | | | | >128 | | | |
| 51 | 1 | 0.25 | >128 | >128 | | | | | 4 | | | |
| 52 | 1 | 0.125 | 32 | >128 | 0.125 | 4 | >128 | 4 | 4 | >128 | >128 | 0.125 |
| 53 | 1 | 0.25 | >128 | >128 | | | | | >128 | | | |
| 54 | 0.5 | 0.125 | >128 | >128 | | | | | 4 | | | |
| 55 | 1 | 0.125 | 8 | >128 | 0.125 | 0.5 | 16 | 4 | 1 | 8 | 8 | 0.125 |
| 56 | 1 | 0.25 | 8 | >128 | 0.25 | 0.5 | 8 | 2 | 1 | 4 | 4 | 0.125 |
| 57 | 0.5 | 0.125 | 8 | >128 | 0.125 | 0.5 | 8 | 4 | 1 | 8 | 8 | 0.125 |
| A | 1-2 | 1-2 | 0.25 | 0.5 | | 0.5 | 0.5 | 0.5 | 0.5 | 0.25 | 0.5 | 0.5 |
| F | >128 | 4 | >128 | >128 | | 8 | >128 | 32 | >128 | >128 | 16 | 64 |
| I | 0.25 | 0.125 | >128 | >128 | | 0.25 | >128 | 0.5 | 0.5 | >128 | 0.125 | 0.125 |

Control compound:
A = amphotericin B,
F = fluconazole,
I = itraconazole

MIC₈₀ of each of the compounds of which antifungal activities can be confirmed by Table 2 was determined using other test strains, i.e., Aspergillus fumigatus ATCC 16424 and MYA-1163, Aspergillus terreus ATCC 28301, Aspergillus flavus ATCC MYA-1004, and Aspergillus niger ATCC 9142. The results are shown in Table 3.

TABLE 3

| | | | | ATCC | |
|---|---|---|---|---|---|
| Ex. | ATCC 16424 | MYA-1163 | MYA-1004 | 28301 | ATCC 9142 |
| 4 | 0.5 | 1 | 1 | 1 | 2 |
| 6 | 0.125 | 0.25 | 0.25 | 0.5 | 1 |
| 12 | 2 | 4 | 4 | 4 | 4 |
| 38 | 0.125 | 0.5 | 0.5 | 1 | 1 |
| A | 0.5 | 2 | 4 | 4 | 0.5 |
| F | >128 | >128 | >128 | >128 | >128 |
| I | 0.125 | 0.5 | 0.25 | 0.25 | 1 |

Control compound:
A = amphotericin B,
F = fluconazole,
I = itraconazole

### TEST EXAMPLE 2

### Antifungal Activity In Vivo

In vivo antifungal activities of the inventive antifungal compounds were evaluated using SPF (specific pathogen free) ICR mice as test animals.

1) Test group: 10 mice were randomly take from healthy male ICR mice. The individual identification was performed by hair marking using a saturated picrinic acid and ID card marking.

2) Administration of immunosuppressive material: for immunosuppression, CPA (200 mg/Kg) was abdominally administered to mice 3 days before fungal infection and test compound administration.

3) Fungal infection: clinical fungi obtained from patients with aspergillosis, Aspergillus fumigatus ATCC 16424 was subcultured in Malt Agar medium for 7 to 10 days, and diluted with 0.85% sterile physiological saline solution (0.2% tween 80) to 5×10⁵ CFU/mL, which was each intravenously administered once to mice in a dose of 0.2 railhead at the compound-administered day.

4) Compound administration

The test and comparative compounds were grinded and diluted with PEG400. The comparative compound, i.e., amphotericin B was dispersed in sterile physiological saline solution, and the test compounds were each dispersed just before administration, deposited at room temperature, and orally administered 2 times in a dose of 50 mg/Kg using a metal sound for oral administration. Amphotericin B was abdominally injected by injection.

5) Administration frequency and period

The initial administrations of test and comparative compounds were each conducted once 2 hrs after fungal infection (day 0). Then, the test compounds were each administered 2 times at every day for 5 days, and the comparative compounds were each abdominally administered once at every day for 5 days.

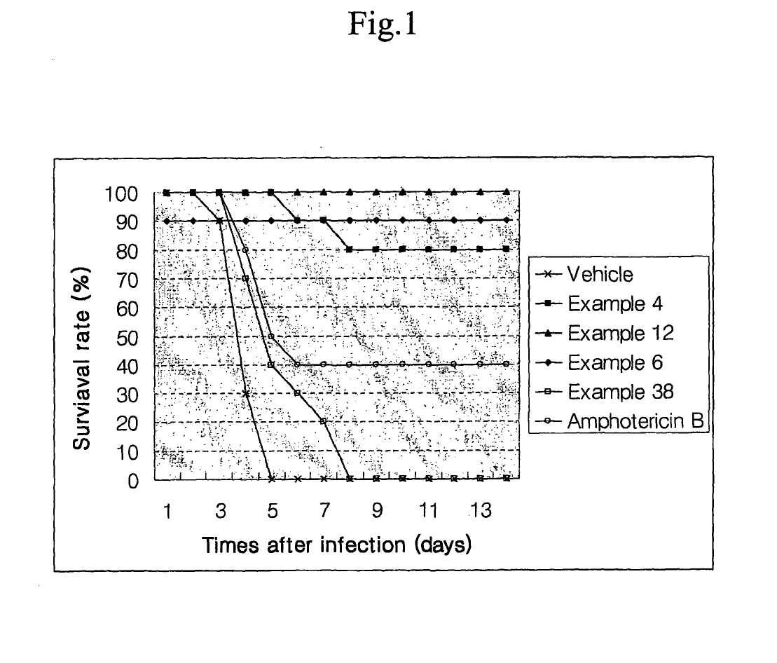

6) Observation for signs of adverse effects or survival rates: all test mice were observed for signs of adverse effects or survival rates (%) at every day for 14 days and the results are shown in FIG. 1.

As shown in FIG. 1, the mice administered with the inventive compounds exhibited high survival rates.

While the invention has been described with respect to the specific embodiments, it should be recognized that various modifications and changes may be made by those skilled in the art to the invention which also fall within the scope of the invention as defined by the appended claims.

What is claimed is:

1. A compound of formula 1 or a pharmaceutically acceptable salt, hydrate, solvate or isomer thereof: 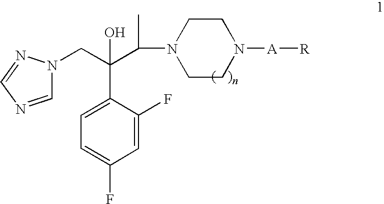
wherein,
n is 1 or 2;
A is a direct bond, C═O or CH₂;
R is a 5 to 10-membered mono- or bi-cyclic heteroaryl ring containing 1 to 4 atoms each independently selected from the group consisting of N, O and S in its ring structure, which is substituted with one or more substituents each independently selected from the group consisting of hydrogen, halogen, hydroxy, cyano, nitro, amino, hydroxycarbonyl, C₁₋₆alkyl, C₁₋₆alkenyl, C₁₋₆alkynyl, C₁₋₆alkoxy, hydroxyl C₁₋₆alkyl, C₁₋₆alkoxy C₁₋₆alkyl, perfluoro C₁₋₆alkyl, perfluoro C₁₋₆alkoxy, C₁₋₆alkylamino, diC₁₋₆alkylamino, aminoC₁₋₆alkyl, C₁₋₆alkylamino C₁₋₆alkyl, diC₁₋₆alkylamino C₁₋₆alkyl, C₁₋₆acyl, C₁₋₆acyloxy, C₁₋₆acyloxyC₁₋₆alkyl, C₁₋₆acylamino, C₁₋₆alkylthio, C₁₋₆alkylthiocarbonyl, C₁₋₆alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl, 3- to 8-membered cycloalkoxy, 3- to 8-membered cycloalkyl-C₁₋₆alkoxy, 3- to 8-membered cycloalkyl-C₁₋₆alkylamino, N—C₁₋₆alkyl N-3- to 8-membered cycloalkyl-C₁₋₆alkylamino, 4- to 8-membered heterocycloalkyl, 4- to 8-membered heterocycloalkyl-C₁₋₆alkoxy, 4- to 8-membered heterocycloalkyl-C₁₋₆alkylamino, N—C₁₋₆alkyl N-4- to 8-membered heterocycloalkyl-C₁₋₆alkylamino, heteroaryl-C₁₋₆alkyl, heteroaryl-C₁₋₆alkoxyl, heteroaryl-C₁₋₆alkylamino, N—C₁₋₆alkyl N-heteroaryl-C₁₋₆alkylamino, phenyl and monocyclic heteroaryl.

2. The compound of claim 1, wherein R is 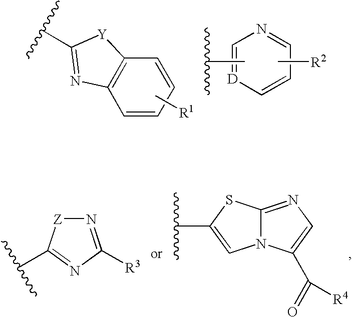
wherein,
Y is O, S, or NR⁵;
D is CH or N;
Z is O or S;
R¹ and R² are each independently hydrogen, halogen, hydroxy, cyano, nitro, amino, hydroxycarbonyl, C₁₋₆alkyl, C₁₋₆alkenyl, C₁₋₆alkoxy, hydroxyC₁₋₆alkyl, C₁₋₆alkoxyC₁₋₆alkyl, perfluoroC₁₋₆alkyl, perfluoroC₁₋₆alkoxy, C₁₋₆alkylamino, diC₁₋₆alkylamino, aminoC₁₋₆alkyl, C₁₋₆alkylaminoC₁₋₆alkyl, diC₁₋₆alkylaminoC₁₋₆alkyl, C₁₋₆acyl, C₁₋₆acyloxy, C₁₋₆acylamino, C₁₋₆alkylthio, C₁₋₆alkylthiocarbonyl, C₁₋₆alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl, 3- to 8-membered cycloalkoxy, 3- to 8-membered cycloalkyl-C₁₋₆alkoxy, 3- to 8-membered cycloalkyl-C₁₋₆alkylamino, N—C₁₋₆alkyl N-3- to 8-membered cycloalkyl-C₁₋₆ alkylamino, 4- to 8-membered heterocycloalkyl, 4- to 8-membered heterocycloalkyl-C₁₋₆alkoxy, 4- to 8-membered heterocycloalkyl-C₁₋₆alkylamino, N—C₁₋₆alkyl N-4- to 8-membered heterocycloalkyl-C₁₋₆alkylamino, heteroaryl-C₁₋₆alkyl, heteroaryl-C₁₋₆alkoxy, heteroaryl-C₁₋₆alkylamino, or N—C₁₋₆alkyl N-heteroaryl-C₁₋₆alkylamino, R³ and R⁴ are each phenyl and monocyclic heteroaryl, substituted with one or more substituents selected from the group consisting of hydrogen, halogen, hydroxy, cyano, nitro, amino, hydroxycarbonyl, C₁₋₆alkyl, C₁₋₆alkenyl, C₁₋₆alkynyl, C₁₋₆alkoxy, hydroxyC₁₋₆alkyl, C₁₋₆alkoxyC₁₋₆alkyl, perfluoroC₁₋₆alkyl and perfluoroC₁₋₆alkoxy; and R⁵ is C₁₋₆alkyl, C₁₋₆alkenyl, C₁₋₆alkynyl, C₁₋₆alkoxyC₁₋₆alkyl or perfluoroC₁₋₆alkyl.

3. The compound of claim 1, which is selected from the groups consisting of the compounds listed in the following Table:

| | |
|---|---|
| 1 | 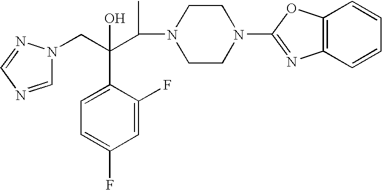 |
| 2 | 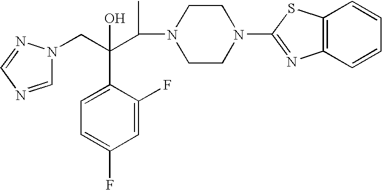 |
| 3 | 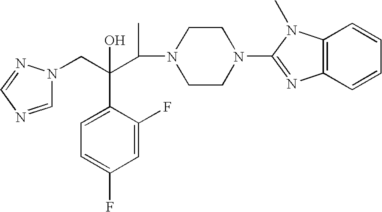 |
| 4 | 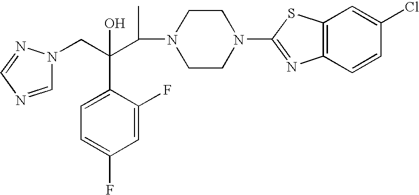 |
| 5 | 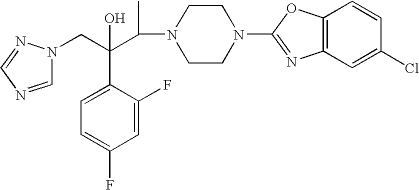 |
| 6 | 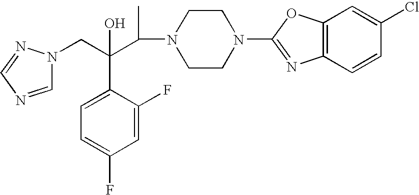 |
| 7 | 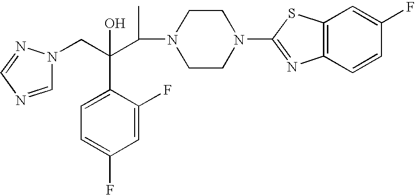 |
| 8 | 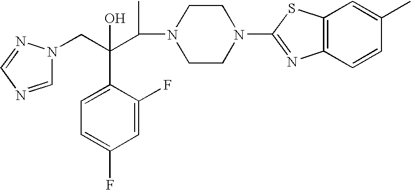 |
| 9 | 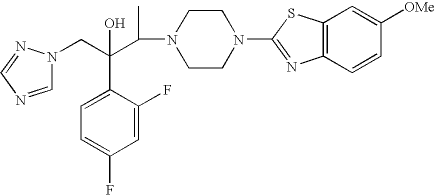 |
| 10 | 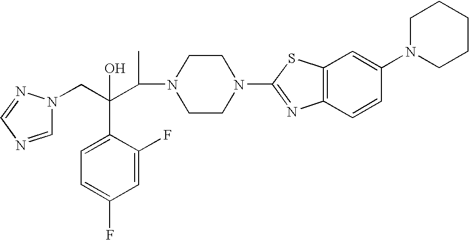 |
| 11 | 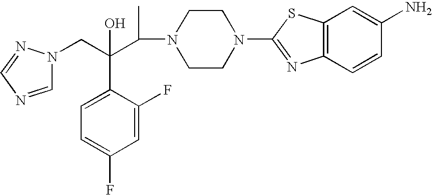 |
| 12 | 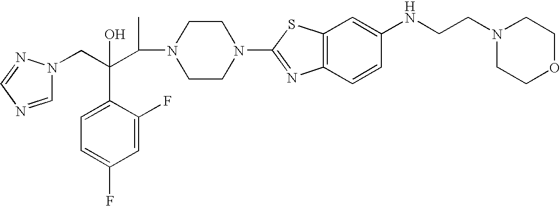 |
| 13 | 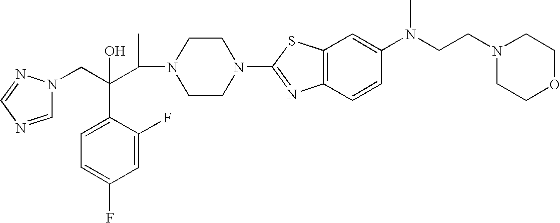 |
| 14 | 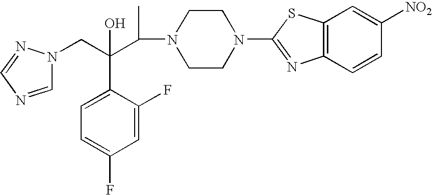 |
| 15 | 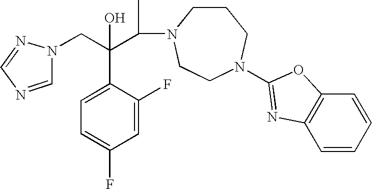 |
| 16 | 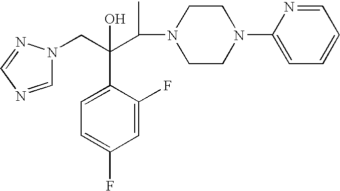 |
| 17 | 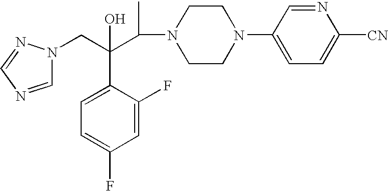 |
| 18 | 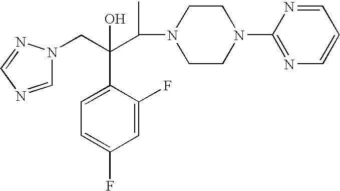 |
| 19 | 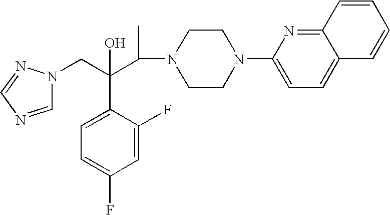 |
| 20 | 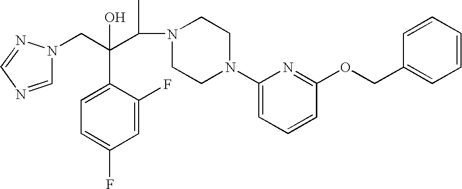 |
| 21 | 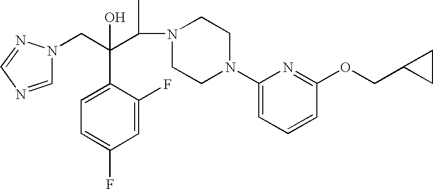 |
| 22 | 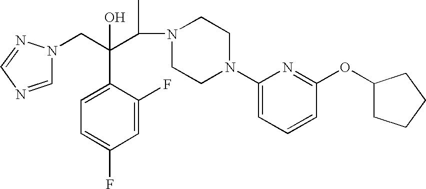 |
| 23 | 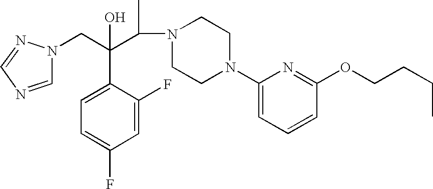 |
| 24 | 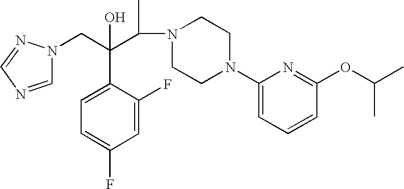 |
| 25 | 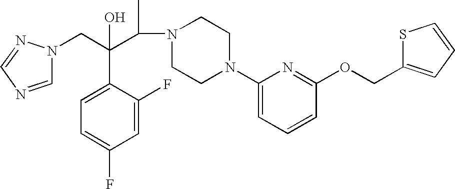 |
| 26 | 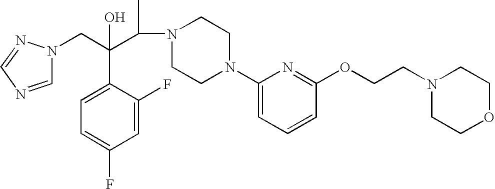 |
| 27 | 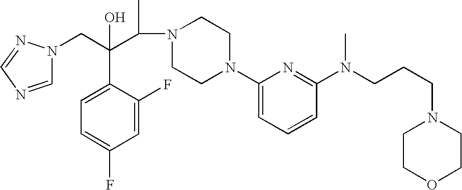 |
| 28 | 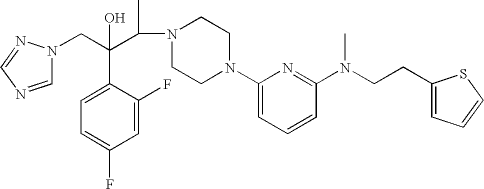 |
| 29 | 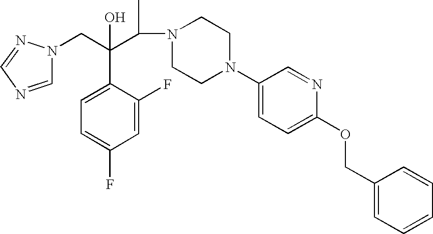 |
| 30 | 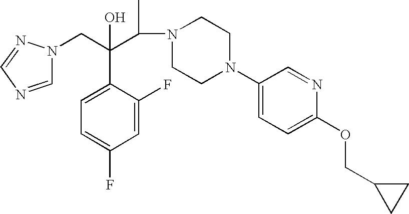 |
| 31 | 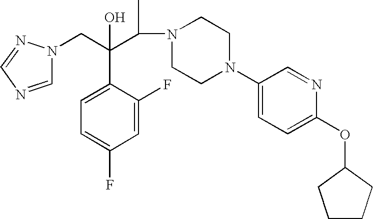 |
| 32 | 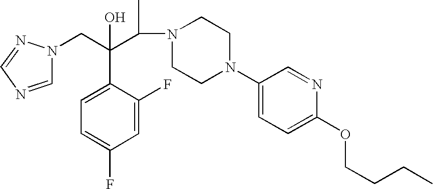 |
| 33 | 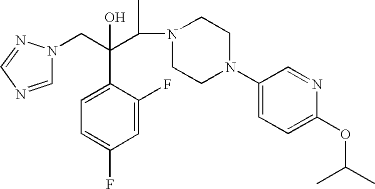 |
| 34 | 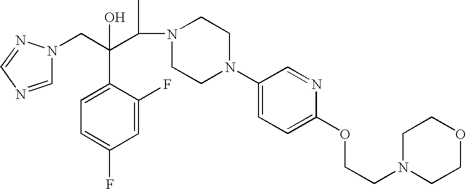 |
| 35 | 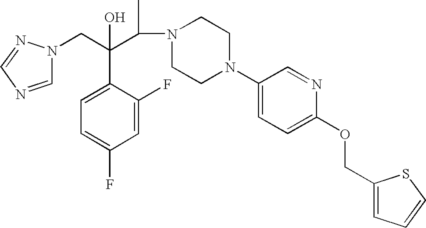 |
| 36 | 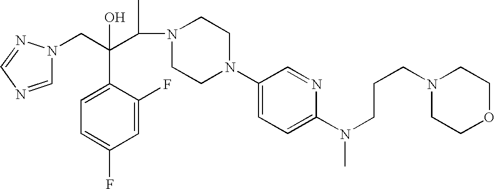 |
| 37 | 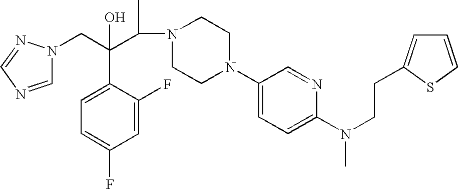 |
| 38 | 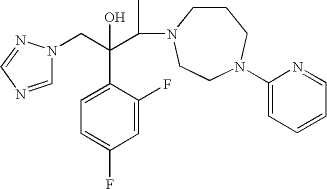 |
| 39 | 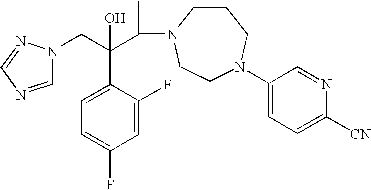 |
| 40 | 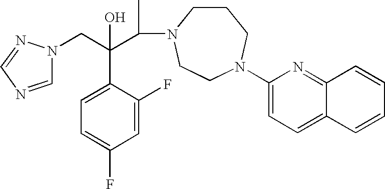 |
| 41 | 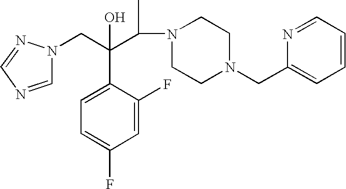 |
| 42 | 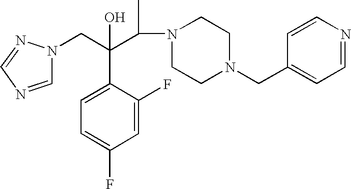 |
| 43 | 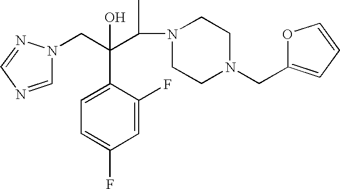 |
| 44 | 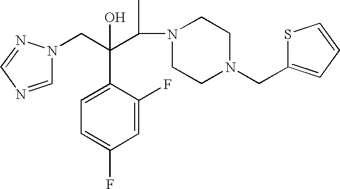 |
| 45 | 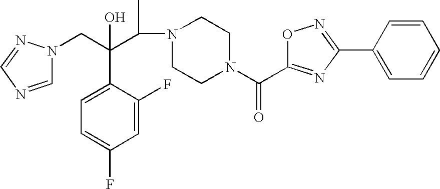 |
| 46 | 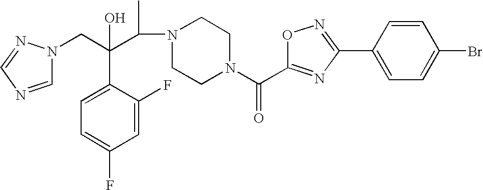 |
| 47 | 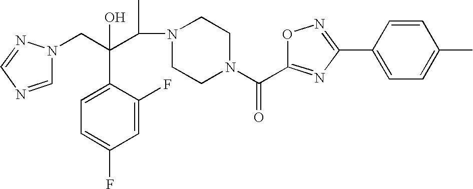 |
| 48 | 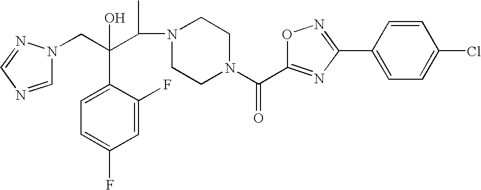 |
| 49 | 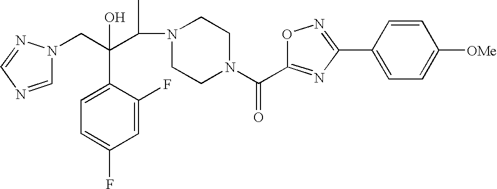 |
| 50 | 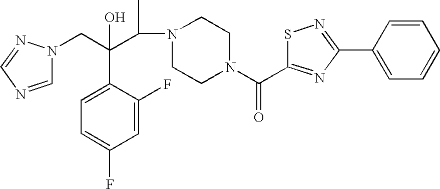 |
| 51 | 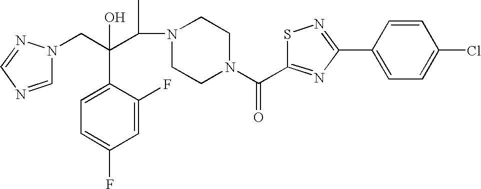 |
| 52 | 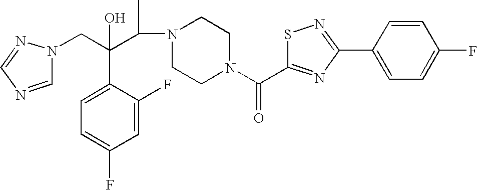 |
| 53 | 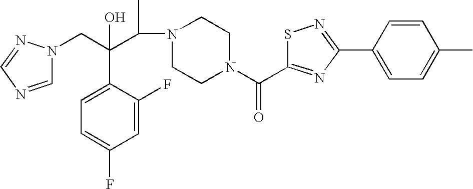 |
| 54 | 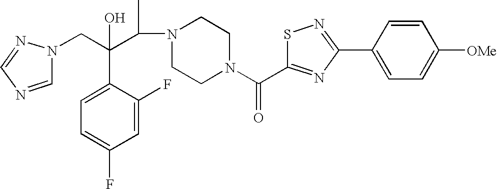 |
| 55 | 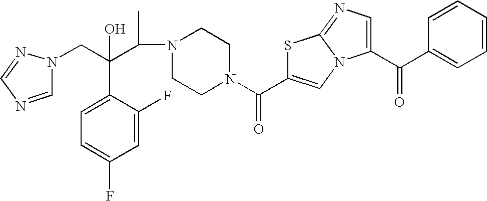 |
| 56 | 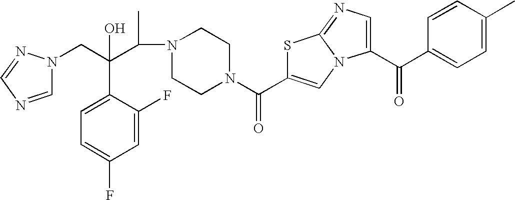 |
| 57 | 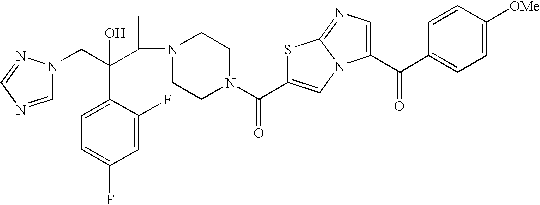 |

4. A method for preparing a compound of formula 1a, comprising the steps of:
1) reacting a compound of formula 2 with a compound of formula 3 in the presence of a base, and optionally removing a protecting group to obtain a compound of formula 5; and
2) reacting the compound of formula 5 with a compound of formula 6: 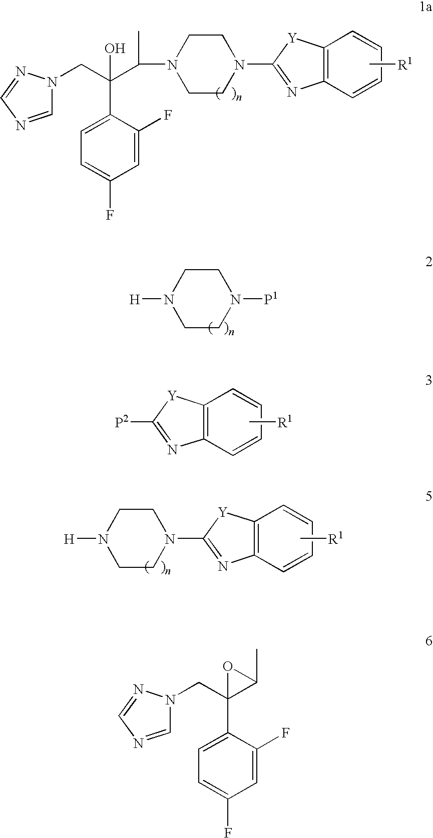
wherein,
n is 1 or 2;
Y and R¹ have the same meanings as defined in claim 2;
P¹ is hydrogen, ethoxycarbonyl, t-butoxycarbonyl or benzyloxycarbonyl; and
P² is halogen, mercapto, methanesulfonyloxy, or trifluoromethanesulfonyloxy.

5. A method for preparing a compound of formula 1a, comprising the steps of:
1) reacting a compound of formula 6 with a compound of formula 2, and optionally removing a protecting group to obtain a compound of formula 8; and
2) reacting the compound of formula 8 with a compound of formula 3: 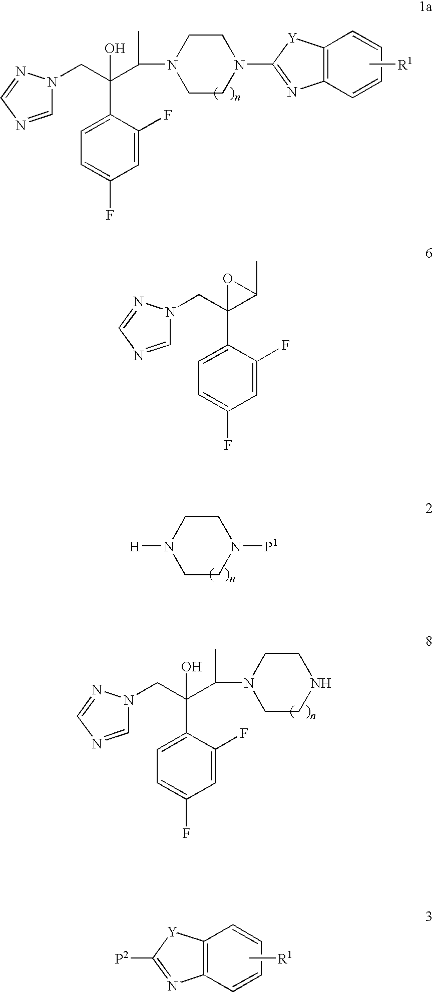
wherein,
n is 1 or 2;
Y and R¹ have the same meanings as defined in claim 2;
P¹ is hydrogen, ethoxycarbonyl, t-butoxycarbonyl or benzyloxycarbonyl; and
P² is halogen, mercapto, methanesulfonyloxy, or trifluoromethanesulfonyloxy.

6. A method for preparing a compound of formula 1b, comprising the steps of
1) reacting a compound of formula 16 with a compound of formula 2, and optionally removing a protecting group to obtain a compound of formula 18; and
2) reacting the compound of formula 18 with a compound of formula 6: 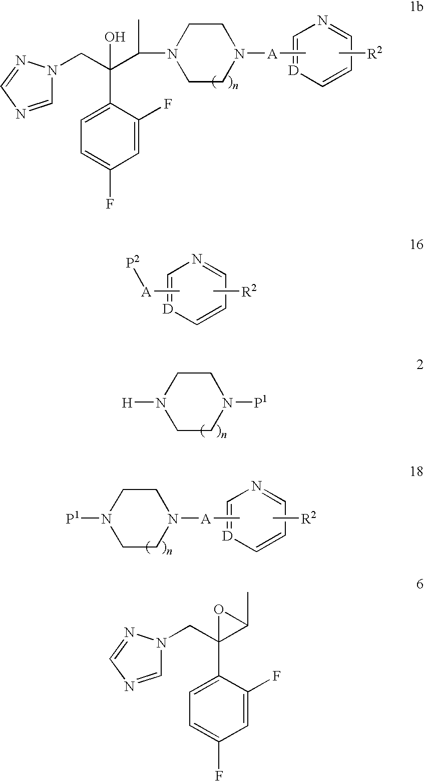
wherein,
n is 1 or 2;
A is a direct bond, C═O or CH₂;
D and R² have the same meanings as defined in claim 2;
P¹ is hydrogen, ethoxycarbonyl, t-butoxycarbonyl or benzyloxycarbonyl; and
P² is halogen, mercapto, methanesulfonyloxy, or trifluoromethanesulfonyloxy.

7. A method for preparing a compound of formula 1c-1, comprising the steps of:
1) reacting a compound of formula 19 with hydroxylamine in the presence of a base to obtain a compound of formula 20;
2) reacting the compound of formula 20 with ethyl chlorooxoacetate to obtain a compound of formula 21; and
3) reacting the compound of formula 21 with a compound of formula 8: 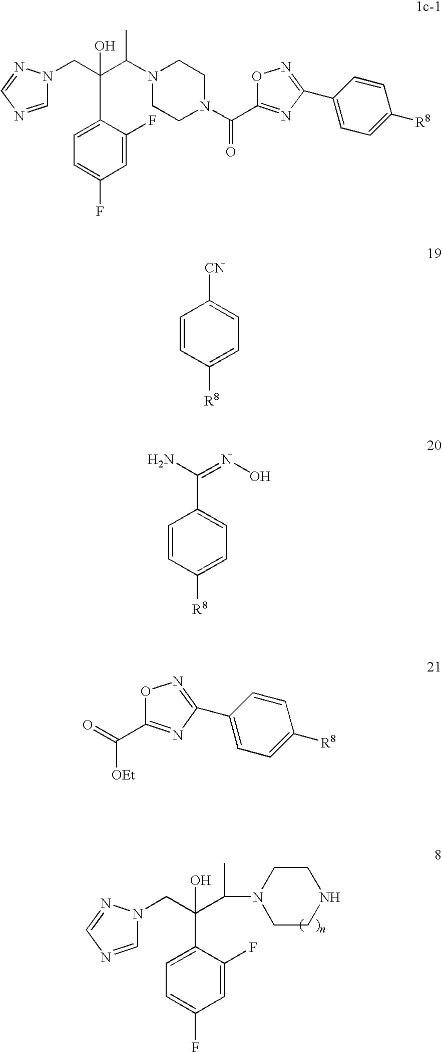
wherein,
n is 1 or 2; and
R⁸ is hydrogen, halogen, hydroxy, C₁₋₆alkoxy cyano, nitro, amino, hydroxycarbonyl, C₁₋₆alkyl, C₁₋₆alkynyl, C₁₋₆alkoxy, hydroxyC₁₋₆alkyl, C₁₋₆alkoxyC₁₋₆alkyl, perfluoroC₁₋₆alkyl, perfluoroC₁₋₆alkoxy, C₁₋₆alkylamino, diC₁₋₆alkylamino, aminoC₁₋₆alkyl, diC₁₋₆alkylaminoC₁₋₆alkyl, C₁₋₆acyl, C₁₋₆acyloxy, C₁₋₆acyloxyC₁₋₆alkyl, C₁₋₆acylamino, C₁₋₆alkylthio, C₁₋₆alkylthiocarbonyl, C₁₋₆alkylthioxo, C₁₋₆alkoxycarbonyl, C₁₋₆alkylsulfonyl, C₁₋₆alkylsulfonylamino, aminosulfonyl, C₁₋₆alkylaminosulfonyl, diC₁₋₆alkylaminosulfonyl, 3- to 8-membered cycloalkyl or 4- to 8-membered heterocycloalkyl.

8. A method for preparing a compound of formula 1c-2, comprising the steps of:
1) reacting a compound of formula 22 with chlorocarbonylsulfenyl chloride to obtain a compound of formula 23;
2) reacting the compound of formula 23 with ethyl cyanoformate to obtain a compound of formula 24; and
3) reacting the compound of formula 24 with a compound of formula 8: 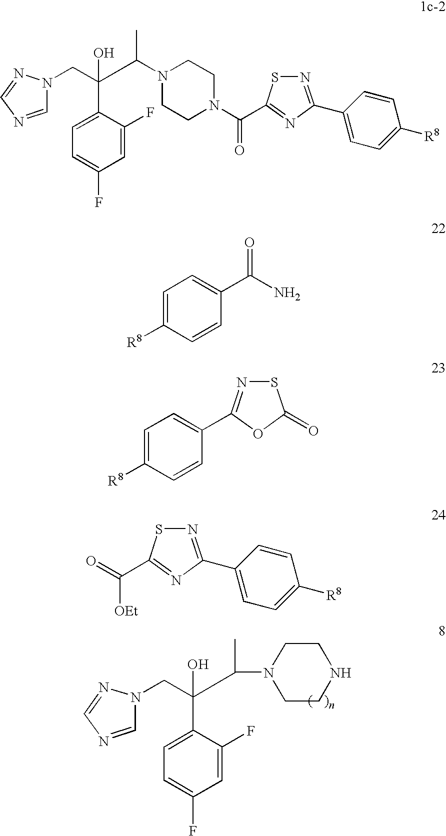
wherein, n is 1 or 2, and R⁸ has the same meaning as defined in claim 7.

9. A method for preparing a compound of formula 1d, comprising the steps of
1) hydrolizing a compound of formula 28 to obtain a compound of formula 29; and
2) reacting the compound of formula 29 with a compound of formula 8 using a coupling agent: 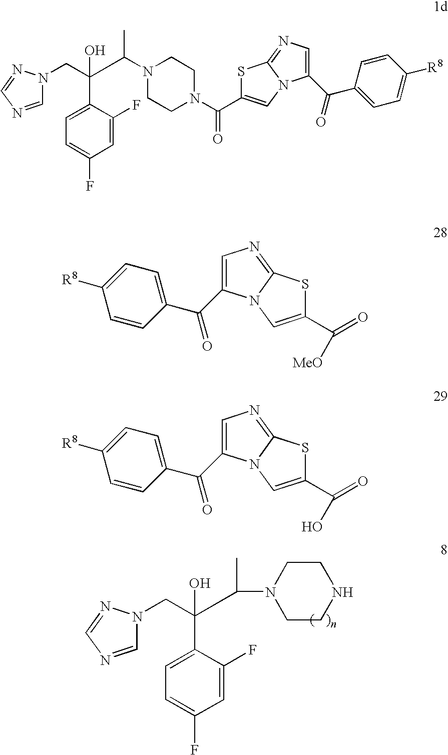
wherein, n is 1 or 2, and R⁸ has the same meaning as defined in claim 7.

10. A pharmaceutical composition for treating diseases caused by fungal infection, comprising the compound of claim 1 or the pharmaceutically acceptable salt, hydrate, solvate or isomer as an active ingredient.
